SEQUENCE LISTING (12 sequences)

SEQ ID NO: 1 - DNA, 1317 bp, synthetic construct
  misc_feature 1..1317: note = Human MYC cDNA sequence
  source 1..1317: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 2 - protein, 439 aa, synthetic construct
  REGION 1..439: note = Human MYC amino acid sequence
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 3 - DNA, 11490 bp, synthetic construct
  misc_feature 1..11490: note = Human TRRAP cDNA sequence
  source 1..11490: mol_type = other DNA
atggcgtttgttgcaacacagggggccacggtggttgaccagaccactttgatgaaaaagtaccttcagtttgtggcagctctcacagatgtgaatacacctgatgaaacaaagttgaaaatgatgcaagaagttagtgaaaattttgagaatgtcacgtcatctcctcagtattctacattcctagaacatatcatccctcgattccttacatttctccaagatggagaagttcagtttcttcaggagaaaccagcacagcaactgcggaagctcgtacttgaaataattcatagaataccaaccaacgaacatcttcgtcctcacacaaaaaatgttttgtctgtgatgtttcgctttttagagacggaaaatgaagaaaatgttcttatttgtctaagaataattattgagctacacaaacagttcaggccaccgatcacacaagaaattcatcattttctggattttgtgaaacagatttacaaggagcttccaaaagtagtgaaccgctactttgagaaccctcaagtgatccccgagaacacagtgcctcccccagaaatggttggtatgataacaacgattgctgtgaaagtcaacccggagcgtgaggacagtgagactcgaacacattccatcattccgaggggatcactttctctgaaagtgttggcagaattgcccattattgttgttttaatgtatcagctctacaaactgaacatccacaatgttgttgctgagtttgtgcccttgatcatgaacaccattgccattcaggtgtctgcacaagcgaggcaacataagctttacaacaaggagttgtatgctgacttcattgctgctcagattaaaacattgtcatttttagcttacattatcaggatttaccaggagttggtgactaagtattctcagcagatggtgaaaggaatgctccagttactttcaaattgtccagcagagactgcacacctcagaaaggagcttctgattgctgccaaacacatcctcaccacagagctgagaaaccagttcattccttgcatggacaagctgtttgatgaatccatactaattggctcaggatatactgccagagagactctaaggcccctcgcctacagcacgctggccgacctcgtgcaccatgtccgccagcacctgcccctcagcgacctctccctcgccgtccagctcttcgccaagaacatcgacgatgagtccctgcccagcagcatccagaccatgtcctgcaagctcctgctgaacctggtggactgcatccgttccaagagcgagcaggagagtggcaatgggagagacgtcctgatgcggatgctggaggttttcgttctcaaattccacacaattgctcggtaccagctctctgccatttttaagaagtgtaagcctcagtcagaacttggagccgtggaagcagctctgcctggggtgcccactgcccctgcagctcctggccctgctccctccccagcccctgtccctgccccacctccacccccgcccccacccccacctgccacccctgtgaccccggcccccgtgcctcccttcgagaagcaaggagaaaaggacaaggaagacaagcagacattccaagtcacagactgtcgaagtttggtcaaaaccttggtgtgtggtgtcaagacaatcacgtggggcataacatcatgcaaagcacctggtgaagctcagttcattcccaacaagcagttacaacccaaagagacacagatttacatcaaacttgtgaaatatgcaatgcaagctttagatatttatcaggtccagatagcaggaaatggacagacatacatccgtgtggccaactgccagactgtgagaatgaaagaggagaaggaggtattggagcatttcgctggtgtgttcacaatgatgaaccccttaacgttcaaagaaatcttccaaactacggtcccttatatggtggagagaatctcaaaaaattatgctcttcagattgttgccaattccttcttggcaaatcctactacctctgctctgtttgctacgattctggtggaatatctccttgatcgcctgccagaaatgggctccaacgtggagctctccaacctgtacctcaagctgttcaagctggtctttggctctgtctccctctttgcagctgaaaatgaacaaatgctgaagcctcacttgcacaagattgtgaacagctctatggagctcgcgcagactgccaaggaaccctacaactacttcttgctgctacgggcgctgtttcgctctattggtggaggtagccacgatctcttgtatcaggagttcttgcctctccttccaaacctcctgcaagggctgaacatgcttcagagtggcctgcacaagcagcacatgaaggacctctttgtggagctgtgtctcaccgtccctgtgcggctgagctcgcttttgccgtacctgcccatgcttatggatcccttggtgtctgcactcaatgggtctcagacattggtcagccaaggcctcaggacgctggagctgtgtgtggacaacctgcagcccgacttcctctacgaccacatccagccggtgcgcgcagagctcatgcaggctctgtggcgcaccttacgcaaccctgctgacagcatctcccacgtggcctaccgtgtgctcggtaagtttggcggcagtaacaggaagatgctgaaggagtcgcagaagctgcactacgttgtgaccgaggttcagggccccagcatcactgtggagttttccgactgcaaagcttctctccagctccccatggagaaggccattgaaactgctctggactgcctgaaaagcgccaacactgagccctactaccggaggcaggcgtgggaagtgatcaaatgcttcctggtggccatgatgagcctggaggacaacaagcacgcactctaccagctcctggcacaccccaactttacagaaaagaccatccccaatgttatcatctcacatcgctacaaagcccaggacactccagcccggaagacttttgagcaggccctgacaggcgccttcatgtctgctgtcattaaggacctgcggcccagcgccctgccctttgtcgccagcttgatccgccactatacgatggtggcagtcgcccagcagtgtggccctttcttgctgccttgctaccaggtgggcagccagcccagcacagccatgtttcacagtgaagaaaatggctcgaaaggaatggatcctttggttctcattgatgcaattgctatttgtatggcatatgaagaaaaggagctttgcaaaatcggggaggtggccctagctgtgatatttgatgttgcaagtatcatcctgggctccaaggagagggcctgccagctgcccctgttttcttacatcgtggagcgcctgtgtgcatgttgttatgaacaggcgtggtatgcaaagctggggggtgtggtgtctattaagtttctcatggagcggctgcctctcacttgggttctccagaaccagcagacattcctgaaagcacttctctttgtcatgatggacttaactggagaggtttccaatggggcagtcgctatggcaaagaccacgctggagcagcttctgatgcggtgcgcaacgcctttaaaagacgaggagagagccgaagagatcgtggccgcccaggaaaagtctttccaccatgtgacacacgacttggttcgagaagtcacctctccaaactccactgtgaggaagcaggccatgcattcgctgcaggtgttggcccaggtcactgggaagagtgtcacggtgatcatggaaccccacaaagaggtcctgcaggatatggtcccccctaagaagcacctgctccgacaccagcctgccaacgcacagattggcctgatggaggggaacacgttctgtaccacgttgcagcccaggctcttcacaatggaccttaacgtggtggagcataaggtgttctacacagagctgttgaatttgtgtgaggctgaagattcagctttaacaaagctgccctgttataaaagccttccgtcactcgtacctttacgaattgcggcattaaatgcacttgctgcctgcaattaccttcctcagtccagggagaaaatcatcgctgcactcttcaaagccctgaattccaccaatagtgagctccaagaggccggagaagcctgtatgagaaagtttttagaaggtgctaccatagaagtcgatcaaatccacacacatatgcgacctttgctgatgatgctgggagattaccggagcttgacgctgaatgttgtgaatcgcctgacttcggtcacgaggctcttcccaaattccttcaatgataaattttgtgatcagatgatgcaacatctgcgcaagtggatggaagtggtggtgatcacccacaaagggggccagaggagcgacggaaacgaaatgaagatttgctcagcaattataaacctttttcatctgatcccggctgctcctcagacactggtgaagcctttgctagaggttgtcatgaaaacggagcgggcgatgctgatcgaggcggggagtccattccgagagcccctgatcaagttcctgactcgacatccctcgcagacagtggagctgttcatgatggaagccacactgaacgatccccagtggagcagaatgtttatgagttttttaaaacacaaagacgccagacctctgcgggatgtgctggctgccaaccccaacaggttcatcaccctgctgctgccggggggtgcccagacggctgtgcgccccggttcgcccagcaccagcaccatgcgcctggacctccagttccaggccatcaagatcataagcattatagtgaaaaacgatgactcctggctggccagccagcactctctggtgagccagttgcgacgtgtgtgggtgagtgagaacttccaagagaggcaccgcaaggagaacatggcagccaccaactggaaggagcccaagctgctggcctactgcctgctgaactactgcaaaaggaattacggagatatagaattgctgttccagctgctccgagcctttactggtcgttttctctgcaacatgacattcttaaaagagtatatggaggaagagattcccaaaaattacagcatcgctcagaaacgtgccctgttctttcgctttgtagacttcaacgaccccaacttcggagatgaattaaaagctaaagttctgcagcatatcttgaatcctgctttcttgtacagctttgagaagggggaaggagagcagctcttgggacctcccaatccagaaggagataacccagaaagcatcaccagtgtgtttattaccaaggtcctggaccccgagaagcaggcggacatgctggactcgctgcggatctacctgctgcagtacgccacgctgctggtggagcacgccccccaccacatccatgacaacaacaagaaccgcaacagcaagctgcgccgcctcatgaccttcgcctggccctgcctgctctccaaggcctgcgtggacccagcctgcaagtacagcggacacttgctcctggcgcacattatcgccaaattcgccatacacaagaagatcgtcctgcaggtttttcatagtctcctcaaggctcacgcaatggaagctcgagcgatcgtcagacaggcgatggccattctgaccccggcggtgccggccaggatggaggacgggcaccagatgctgacccactggacccggaagatcattgtggaggaggggcacaccgtcccgcagctggtccacattctgcacctgatagtgcaacacttcaaggtgtactacccggtacggcaccacttggtgcagcacatggtgagcgccatgcagaggctgggcttcacgcccagtgtcaccatcgagcagaggcggctggccgtggacctgtctgaagtcgtcatcaagtgggagctgcagaggatcaaggaccagcagccggattcagatatggacccaaattccagtggagaaggagtcaattctgtctcatcctccattaagagaggcctgtccgtggattctgcccaggaagtgaaacgctttaggacggccaccggagccatcagtgcagtctttgggaggagccagtcgctacctggagcagactctctcctcgccaagcccattgacaagcagcacacagacactgtggtgaacttccttatccgcgtggcctgtcaggttaatgacaacaccaacacagcggggtcccctggggaggtgctctctcgccggtgtgtgaaccttctgaagactgcgttgcggccagacatgtggcccaagtccgaactcaagctgcagtggttcgacaagctgctgatgactgtggagcagccaaaccaagtgaactatgggaatatctgcacgggcctagaagtgctgagcttcctgctaactgtcctccagtccccagccatcctcagtagcttcaaacctctgcagcgtggaattgccgcctgcatgacatgtggaaacaccaaggtgttgcgagccgtccacagccttctctcgcgcctgatgagcattttcccaacagagccgagtacttccagtgtggcctccaaatatgaagagctggagtgcctctacgcagccgtcggaaaggtcatctatgaagggctcaccaactacgagaaggccaccaatgccaatccctcccagctcttcgggacccttatgatcctcaagtctgcctgcagcaacaaccccagctacatagacaggctgatctccgtctttatgcgctccctgcagaagatggtccgggagcatttaaaccctcaggcagcgtcaggaagcaccgaagccacctcaggtacaagcgagctggtgatgctgagtctggagctggtgaagacgcgcctggcagtgatgagcatggagatgcggaagaacttcatccaggccatcctgacatccctcatcgaaaaatcaccagatgccaaaatcctccgggctgtggtcaaaatcgtggaagaatgggtcaagaataactccccaatggcagccaatcagacacctacactccgggagaagtccattttgcttgtgaagatgatgacttacatagaaaaacgctttccggaagaccttgaattaaatgcccagtttttagatcttgttaactatgtctacagggatgagaccctctctggcagcgagctgacggcgaaacttgagcctgcctttctctctgggctgcgctgtgcccagccactcatcagggcaaagtttttcgaggtttttgacaactccatgaaacgtcgtgtctacgagcgcttgctctatgtgacctgttcgcagaactgggaagccatggggaaccacttctggatcaagcagtgcattgagctgcttctggccgtgtgtgagaagagcacccccattggcaccagctgccaaggagccatgctcccgtccatcaccaacgtcatcaacctggccgatagccacgaccgtgccgccttcgccatggtcacacatgtcaagcaggagccccgggagcgggagaacagcgagtccaaagaggaggatgtagagatagacatcgaactagctcctggggatcagaccagcacgcccaaaaccaaagaactttcagaaaaggacattggaaaccagctgcacatgctaaccaacaggcacgacaagtttctggacactctccgagaggtgaagactggagcgctgctcagcgctttcgttcagctgtgccacatttccacgacgctggcagagaagacgtgggtccagcttttccccagattgtggaagatcctctctgacagacagcagcatgcactcgcgggtgagataagtccatttctgtgcagcggcagtcaccaggtgcagcgggactgccagcccagcgcgctgaactgctttgtggaagccatgtcccagtgcgtgccgccaatccccatccgaccctgcgtcctgaagtacctggggaagacacacaacctctggttccggtccacgctgatgttggagcaccaggcttttgaaaagggtctgagtcttcagattaagccgaagcaaacaacggagttttatgagcaggagagcatcaccccgccgcagcaggagatactggattcccttgcggagctttactccctgttacaagaggaagatatgtgggctggtctgtggcagaagcggtgcaagtactcggagacagcgactgcgattgcttacgagcagcacgggttctttgagcaggcacaagaatcctatgaaaaggcaatggataaagccaaaaaagaacatgagaggagtaacgcctcccctgctattttccctgaataccagctctgggaagaccactggattcgatgctccaaggaattgaaccagtgggaagccctgacggagtacggtcagtccaaaggccacatcaacccctacctcgtcctggagtgcgcctggcgggtgtccaactggactgccatgaaggaggcgctggtgcaggtggaagtgagctgtccgaaggagatggcctggaaggtgaacatgtaccgcggatacctggccatctgccaccccgaggagcagcagctcagcttcatcgagcgcctggtggagatggccagcagcctggccatccgcgagtggcggcggctgccccacgtagtgtcccacgtgcacacgcctctcctacaggcagcccagcaaatcatcgaactccaggaagctgcacaaatcaacgcaggcttacagccaaccaacctgggaaggaacaacagcctgcacgacatgaagacggtggtgaagacctggaggaaccgactgcccatcgtgtctgacgacttgtcccactggagcagcatcttcatgtggaggcagcatcattaccaggcgattgtaactgcctatgagaatagctctcagcatgatcccagttcaaataacgctatgcttggggttcatgcatcagcttcagcgatcatccagtatggaaaaatcgcccggaaacaaggactggtcaatgtagctctggatatattaagtcggattcatactattccaactgttcctatcgtggattgcttccagaagattcgacagcaagttaaatgctacctccagctggcaggcgtcatgggcaaaaacgagtgcatgcagggccttgaagttattgaatctacaaatttaaaatacttcacaaaagagatgacagccgaattttatgcactgaagggaatgttcttggctcagatcaacaagtccgaggaggcaaacaaagccttctctgcagctgtgcagatgcacgatgtgctggtgaaagcctgggccatgtggggcgactacctggagaacatctttgtgaaggagcggcagctgcacctgggcgtgtctgccatcacctgctacctgcacgcctgccggcatcagaacgagagcaaatcgaggaaatacttagccaaggtgctgtggcttttgagttttgatgatgacaaaaacactttggcagatgccgtcgacaagtactgcattggtgtgccacccatccagtggctggcctggatcccacagctgctcacctgcctggttggctcggagggaaagctgctcttgaacctcattagccaggttggacgcgtgtatccccaagcggtctactttcccatccggaccctgtacctgaccctgaaaatagaacagcgggaacgctacaagagcgatccagggcccataagagcaacagcacccatgtggcgctgcagccgaatcatgcacatgcagcgagagctccaccccacccttctgtcttccctggaaggcatcgtcgatcagatggtctggttcagagaaaattggcatgaagaggttctcaggcagctccaacagggcctggcgaaatgttactccgtggcgtttgagaaaagtggagcggtgtccgatgctaaaatcaccccccacactctcaattttgtgaagaagttggtgagcacgtttggggtgggcctggagaatgtgtccaacgtctcgaccatgttctccagcgcagcctctgagtctctggcccggcgggcgcaggccactgcacaagaccctgtctttcagaagctgaaaggccagttcacgacggattttgacttcagcgttccaggatccatgaagcttcataatcttatttctaagttgaaaaagtggatcaaaatcttggaggccaagaccaagcaactccccaaattcttcctcatagaggaaaagtgccggttcttgagcaatttctcggcacagacagctgaagtggaaattcctggggagtttctgatgccaaagccaacgcattattacatcaagattgcacggttcatgccccgggtagagattgtgcagaagcacaacaccgcagcccggcggctgtacatccggggacacaatggcaagatctacccatacctcgtcatgaacgacgcctgcctcacagagtcacggcgagaggagcgtgtgttgcagctgctgcgtctgctgaacccctgtttggagaagagaaaggagaccaccaagaggcacttgtttttcacagtgccccgggttgtggcagtttccccacagatgcgcctcgtggaggacaacccctcttcactttcccttgtggagatctacaagcagcgctgcgccaagaagggcatcgagcatgacaaccccatctcccgttactatgaccggctggctacggtgcaggcgcggggaacccaagccagccaccaggtcctccgcgacatcctcaaggaggttcagagtaacatggtgccgcgcagcatgctcaaggagtgggcgctgcacaccttccccaatgccacggactactggacgttccggaagatgttcaccatccagctggctctgataggcttcgcggaattcgtcctgcatttaaatagactcaaccccgagatgttacagatcgctcaggacactggcaaactgaatgttgcctactttcgatttgacataaacgacgcgactggagacctggatgccaaccgtcctgtcccatttcgactcacgcccaacatttctgagtttctgaccaccatcggggtctccggcccgttgacagcgtccatgattgcggtcgcccggtgcttcgcccagccaaactttaaggtggatggcattctgaaaacggttctccgggacgagatcattgcttggcacaaaaaaacacaagaggacacgtcctctcctctctcggccgccgggcagccagagaacatggacagccagcaactggtgtccctggttcagaaagccgtcaccgccatcatgacccgcctgcacaacctcgcccagttcgaaggcggggaaagcaaggtgaacaccctggtggccgcggcaaacagcctggacaatctgtgccgcatggaccccgcctggcacccctggctg

SEQ ID NO: 4 - protein, 3830 aa, synthetic construct
  REGION 1..3830: note = Human TRRAP amino acid sequence
MAFVATQGATVVDQTTLMKKYLQFVAALTDVNTPDETKLKMMQEVSENFENVTSSPQYSTFLEHIIPRFLTFLQDGEVQFLQEKPAQQLRKLVLEIIHRIPTNEHLRPHTKNVLSVMFRFLETENEENVLICLRIIIELHKQFRPPITQEIHHFLDFVKQIYKELPKVVNRYFENPQVIPENTVPPPEMVGMITTIAVKVNPEREDSETRTHSIIPRGSLSLKVLAELPIIVVLMYQLYKLNIHNVVAEFVPLIMNTIAIQVSAQARQHKLYNKELYADFIAAQIKTLSFLAYIIRIYQELVTKYSQQMVKGMLQLLSNCPAETAHLRKELLIAAKHILTTELRNQFIPCMDKLFDESILIGSGYTARETLRPLAYSTLADLVHHVRQHLPLSDLSLAVQLFAKNIDDESLPSSIQTMSCKLLLNLVDCIRSKSEQESGNGRDVLMRMLEVFVLKFHTIARYQLSAIFKKCKPQSELGAVEAALPGVPTAPAAPGPAPSPAPVPAPPPPPPPPPPATPVTPAPVPPFEKQGEKDKEDKQTFQVTDCRSLVKTLVCGVKTITWGITSCKAPGEAQFIPNKQLQPKETQIYIKLVKYAMQALDIYQVQIAGNGQTYIRVANCQTVRMKEEKEVLEHFAGVFTMMNPLTFKEIFQTTVPYMVERISKNYALQIVANSFLANPTTSALFATILVEYLLDRLPEMGSNVELSNLYLKLFKLVFGSVSLFAAENEQMLKPHLHKIVNSSMELAQTAKEPYNYFLLLRALFRSIGGGSHDLLYQEFLPLLPNLLQGLNMLQSGLHKQHMKDLFVELCLTVPVRLSSLLPYLPMLMDPLVSALNGSQTLVSQGLRTLELCVDNLQPDFLYDHIQPVRAELMQALWRTLRNPADSISHVAYRVLGKFGGSNRKMLKESQKLHYVVTEVQGPSITVEFSDCKASLQLPMEKAIETALDCLKSANTEPYYRRQAWEVIKCFLVAMMSLEDNKHALYQLLAHPNFTEKTIPNVIISHRYKAQDTPARKTFEQALTGAFMSAVIKDLRPSALPFVASLIRHYTMVAVAQQCGPFLLPCYQVGSQPSTAMFHSEENGSKGMDPLVLIDAIAICMAYEEKELCKIGEVALAVIFDVASIILGSKERACQLPLFSYIVERLCACCYEQAWYAKLGGVVSIKFLMERLPLTWVLQNQQTFLKALLFVMMDLTGEVSNGAVAMAKTTLEQLLMRCATPLKDEERAEEIVAAQEKSFHHVTHDLVREVTSPNSTVRKQAMHSLQVLAQVTGKSVTVIMEPHKEVLQDMVPPKKHLLRHQPANAQIGLMEGNTFCTTLQPRLFTMDLNVVEHKVFYTELLNLCEAEDSALTKLPCYKSLPSLVPLRIAALNALAACNYLPQSREKIIAALFKALNSTNSELQEAGEACMRKFLEGATIEVDQIHTHMRPLLMMLGDYRSLTLNVVNRLTSVTRLFPNSFNDKFCDQMMQHLRKWMEVVVITHKGGQRSDGNEMKICSAIINLFHLIPAAPQTLVKPLLEVVMKTERAMLIEAGSPFREPLIKFLTRHPSQTVELFMMEATLNDPQWSRMFMSFLKHKDARPLRDVLAANPNRFITLLLPGGAQTAVRPGSPSTSTMRLDLQFQAIKIISIIVKNDDSWLASQHSLVSQLRRVWVSENFQERHRKENMAATNWKEPKLLAYCLLNYCKRNYGDIELLFQLLRAFTGRFLCNMTFLKEYMEEEIPKNYSIAQKRALFFRFVDFNDPNFGDELKAKVLQHILNPAFLYSFEKGEGEQLLGPPNPEGDNPESITSVFITKVLDPEKQADMLDSLRIYLLQYATLLVEHAPHHIHDNNKNRNSKLRRLMTFAWPCLLSKACVDPACKYSGHLLLAHIIAKFAIHKKIVLQVFHSLLKAHAMEARAIVRQAMAILTPAVPARMEDGHQMLTHWTRKIIVEEGHTVPQLVHILHLIVQHFKVYYPVRHHLVQHMVSAMQRLGFTPSVTIEQRRLAVDLSEVVIKWELQRIKDQQPDSDMDPNSSGEGVNSVSSSIKRGLSVDSAQEVKRFRTATGAISAVFGRSQSLPGADSLLAKPIDKQHTDTVVNFLIRVACQVNDNTNTAGSPGEVLSRRCVNLLKTALRPDMWPKSELKLQWFDKLLMTVEQPNQVNYGNICTGLEVLSFLLTVLQSPAILSSFKPLQRGIAACMTCGNTKVLRAVHSLLSRLMSIFPTEPSTSSVASKYEELECLYAAVGKVIYEGLTNYEKATNANPSQLFGTLMILKSACSNNPSYIDRLISVFMRSLQKMVREHLNPQAASGSTEATSGTSELVMLSLELVKTRLAVMSMEMRKNFIQAILTSLIEKSPDAKILRAVVKIVEEWVKNNSPMAANQTPTLREKSILLVKMMTYIEKRFPEDLELNAQFLDLVNYVYRDETLSGSELTAKLEPAFLSGLRCAQPLIRAKFFEVFDNSMKRRVYERLLYVTCSQNWEAMGNHFWIKQCIELLLAVCEKSTPIGTSCQGAMLPSITNVINLADSHDRAAFAMVTHVKQEPRERENSESKEEDVEIDIELAPGDQTSTPKTKELSEKDIGNQLHMLTNRHDKFLDTLREVKTGALLSAFVQLCHISTTLAEKTWVQLFPRLWKILSDRQQHALAGEISPFLCSGSHQVQRDCQPSALNCFVEAMSQCVPPIPIRPCVLKYLGKTHNLWFRSTLMLEHQAFEKGLSLQIKPKQTTEFYEQESITPPQQEILDSLAELYSLLQEEDMWAGLWQKRCKYSETATAIAYEQHGFFEQAQESYEKAMDKAKKEHERSNASPAIFPEYQLWEDHWIRCSKELNQWEALTEYGQSKGHINPYLVLECAWRVSNWTAMKEALVQVEVSCPKEMAWKVNMYRGYLAICHPEEQQLSFIERLVEMASSLAIREWRRLPHVVSHVHTPLLQAAQQIIELQEAAQINAGLQPTNLGRNNSLHDMKTVVKTWRNRLPIVSDDLSHWSSIFMWRQHHYQAIVTAYENSSQHDPSSNNAMLGVHASASAIIQYGKIARKQGLVNVALDILSRIHTIPTVPIVDCFQKIRQQVKCYLQLAGVMGKNECMQGLEVIESTNLKYFTKEMTAEFYALKGMFLAQINKSEEANKAFSAAVQMHDVLVKAWAMWGDYLENIFVKERQLHLGVSAITCYLHACRHQNESKSRKYLAKVLWLLSFDDDKNTLADAVDKYCIGVPPIQWLAWIPQLLTCLVGSEGKLLLNLISQVGRVYPQAVYFPIRTLYLTLKIEQRERYKSDPGPIRATAPMWRCSRIMHMQRELHPTLLSSLEGIVDQMVWFRENWHEEVLRQLQQGLAKCYSVAFEKSGAVSDAKITPHTLNFVKKLVSTFGVGLENVSNVSTMFSSAASESLARRAQATAQDPVFQKLKGQFTTDFDFSVPGSMKLHNLISKLKKWIKILEAKTKQLPKFFLIEEKCRFLSNFSAQTAEVEIPGEFLMPKPTHYYIKIARFMPRVEIVQKHNTAARRLYIRGHNGKIYPYLVMNDACLTESRREERVLQLLRLLNPCLEKRKETTKRHLFFTVPRVVAVSPQMRLVEDNPSSLSLVEIYKQRCAKKGIEHDNPISRYYDRLATVQARGTQASHQVLRDILKEVQSNMVPRSMLKEWALHTFPNATDYWTFRKMFTIQLALIGFAEFVLHLNRLNPEMLQIAQDTGKLNVAYFRFDINDATGDLDANRPVPFRLTPNISEFLTTIGVSGPLTASMIAVARCFAQPNFKVDGILKTVLRDEIIAWHKKTQEDTSSPLSAAGQPENMDSQQLVSLVQKAVTAIMTRLHNLAQFEGGESKVNTLVAAANSLDNLCRMDPAWHPWL

SEQ ID NO: 5 - protein, 15 aa, synthetic construct
  REGION 1..15: note = Flexible Linker
GSGSAGSAAGSGEFG

SEQ ID NO: 6 - protein, 17 aa, synthetic construct
  REGION 1..17: note = Human MYC 129-145 (MB2) amino acid sequence
IIQDCMWSGFSAAAKLV

SEQ ID NO: 7 - protein, 190 aa, synthetic construct
  REGION 1..190: note = Human MYC 1-190 amino acid sequence
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECID

SEQ ID NO: 8 - protein, 42 aa, synthetic construct
  REGION 1..42: note = Human MYC 120-161 amino acid sequence
DDETFIKNIIIQDCMWSGFSAAAKLVSEKLASYQAARKDSGS

SEQ ID NO: 9 - protein, 56 aa, synthetic construct
  REGION 1..56: note = Human TRRAP 2033-2088 amino acid sequence
SVDSAQEVKRFRTATGAISAVFGRSQSLPGADSLLAKPIDKQHTDTVVNFLIRVAC

SEQ ID NO: 10 - protein, 251 aa, synthetic construct
  REGION 1..251: note = Human TRRAP 2033-2283 amino acid sequence
SVDSAQEVKRFRTATGAISAVFGRSQSLPGADSLLAKPIDKQHTDTVVNFLIRVACQVNDNTNTAGSPGEVLSRRCVNLLKTALRPDMWPKSELKLQWFDKLLMTVEQPNQVNYGNICTGLEVLSFLLTVLQSPAILSSFKPLQRGIAACMTCGNTKVLRAVHSLLSRLMSIFPTEPSTSSVASKYEELECLYAAVGKVIYEGLTNYEKATNANPSQLFGTLMILKSACSNNPSYIDRLISVFMRSLQKMV

SEQ ID NO: 11 - DNA, 480 bp, synthetic construct
  misc_feature 1..480: note = Human MAX cDNA sequence
  source 1..480: mol_type = other DNA
atgagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagc

SEQ ID NO: 12 - protein, 160 aa, synthetic construct
  REGION 1..160: note = Human MAX amino acid sequence
MSDNDDIEVESDEEQPRFQSAADKRAHHNALERKRRDHIKDSFHSLRDSVPSLQGEKASRAQILDKATEYIQYMRRKNHTHQQDIDDLKRQNALLEQQVRALEKARSSAQLQTNYPSSDNSLYTNAKGSTISAFDGGSDSSSESEPEEPQSRKKLRMEAS

(19) United States
(12) Patent Application Publication
(10) Pub. No.: US 2026/0216144 A1
(43) Pub. Date: Jul. 30, 2026

(54) TARGETING MB2 OF THE MYC ONCOGENE AND ITS INTERACTION WITH TRRAP IN CANCER

(71) Applicant: Trustees of Dartmouth College, Hanover, NH (US)

(72) Inventors: Edmond J. Feris, Philadelphia, PA (US); Michael D. Cole, Hanover, NH (US)

(73) Assignee: Trustees of Dartmouth College, Hanover, NH (US)

(21) Appl. No.: 19/644,063

(22) Filed: Apr. 10, 2026

Related U.S. Application Data
(62) Division of application No. 17/781,406, filed on Dec. 2, 2020.
(60) Provisional application No. 62/942,734, filed on Dec. 2, 2019.

(51) Int. Cl. A61K 31/427; A61K 31/4184; A61K 31/4439; A61K 31/4709; A61K 31/501; A61K 31/502; A61K 31/517; A61K 31/519; A61P 35/00; G01N 33/50

(52) U.S. Cl. CPC A61K 31/427; A61K 31/4184; A61K 31/4439; A61K 31/4709; A61K 31/501; A61K 31/502; A61K 31/517; A61K 31/519; A61P 35/00; G01N 33/5023

(57) ABSTRACT

Provided are methods and compositions for identifying inhibitors of an interaction between the oncogenic transcription factor MYC and its cofactor TRRAP. The methods involve both cell-based and in vitro approaches for probing an interaction between MYC and TRRAP and for identifying inhibitors of a MYC-TRRAP interaction. Also provided are compounds for use as inhibitors of an interaction between MYC and TRRAP, and methods for developing a cancer therapeutic from such compounds, including methods for derivatizing such inhibitors and for testing the inhibitors and derivatized inhibitors for an ability to treat cancer in a subject. The methods, compounds, and compositions provided herein can provide various advantages, such as a means to target the oncogenic transcription factor MYC in cancer.

### RELATED APPLICATIONS

This application is a divisional of U.S. application Ser. No. 17/781,406, filed Jun. 1, 2022, which is a national stage filing under 35 U.S.C. § 371 of international patent application number PCT/US2020/062870, filed Dec. 2, 2020, which claims the benefit under 35 U.S.C. § 119(e) of U.S. provisional application No. 62/942,734, filed on Dec. 2, 2019, each of which is incorporated by reference herein in its entirety.

### STATEMENT OF FEDERALLY FUNDED RESEARCH

This invention was made with government support under Grant No. 5R01CA055248-25 awarded by the National Cancer Institute of the National Institutes of Health. The U.S. government has certain rights in the invention.

### REFERENCE TO A SEQUENCE LISTING

The contents of the electronic sequence listing (T063670006US02-SEQ-JXV.xml; Size: 34,399 bytes; and Date of Creation: Apr. 9, 2026) are herein incorporated by reference in their entirety.

### FIELD OF THE ART

The present disclosure generally relates to the field of cancer therapeutics, and more particularly, to methods for identifying an inhibitor of an interaction between MYC and TRRAP. As such, the present disclosure relates to cell-based and in vitro methods and compositions for probing an interaction between MYC and TRRAP. The present disclosure also generally relates to chemical compounds and their derivatives for use as Inhibitors of an interaction between MYC and TRRAP, to therapeutic compositions comprising such inhibitors, and to methods of use thereof for treating cancer in a subject.

### BACKGROUND

Cancer cells evolve through a multistage process, driven by the progressive accumulation of multiple genetic and epigenetic abnormalities. Despite the complexity of carcinogenesis, the process is fragile: the growth and survival of cancerous cells can be impaired by the inactivation of a single oncogene (1). Altered transcriptional programs can also make cancer cells highly dependent on certain regulators of gene expression (2). Therefore, research into mechanisms of cellular proliferation carries the promise of discovering new therapies. Extensive studies of tumor genomes have revealed recurrent somatic mutations that affect normal transcriptional control (2). One of these is MYC, a master regulator of transcription. MYC plays a central role in carcinogenesis and is the most wanted target for drugs that perturb dysregulated transcriptional programs. The fact that many cancercells cannot survive without MYC—a phenomenon termed “MYC addiction”—provides a compelling case for the development of MYC-specific targeted therapies.

### The MYC Transcription Factor

Deregulated expression of MYC is a hallmark of 70% of all cancers (3) and MYC is the most frequently amplified gene in human cancer. Furthermore, a diverse array of mutations in oncogenic signaling pathways can lead to MYC overexpression (4, 5). Relatively small changes in MYC protein levels can promote or block oncogenic transformation or cancer development. The biological functions of MYC may be broader than those of any other gene. These functions include controlling cell proliferation, promoting oncogenic transformation, inducing tumor formation, blocking differentiation, inducing apoptosis, inducing G2 arrest, and altering the inherited predisposition to cancer, among others (6).

The MYC family has three members: c-MYC (MYC), N-MYC (or MYCN) and L-MYC (or MYCL). During the life of an organism, MYC is universally expressed in all proliferating cells, whereas MYCN is often co-expressed with MYC in stem cells and other primitive lineages (7, 8). While MYCN is amplified in a subset of tumors, MYC is the most frequently deregulated gene in cancer (9, 10). Although all three members of the MYC family differ in cellular expression and chromosomal locus, their protein products consist primarily of the same two domains: an N-terminal transactivation domain and a C-terminal DNA binding domain. The C-termini of all MYC family proteins are highly conserved and include a basic helix-loop-helix/leucine zipper (bHLH/LZ) motif, and the basic region is required for sequence-specific interaction with DNA (11,12). The N-termini of MYC family members have four main regions of conserved structure. These regions are referred to as MYC homology boxes 1 through 4 (MB1-4) (13). The MBs, especially MB2, are highly evolutionarily conserved, extending from humans to sponge (14). MB2 is necessary for both transactivation and repression of MYC's ‘classical’ target genes (15-17).

Since MYC has no inherent enzymatic activity, it is sometimes thought to be “undruggable” (18,19). However, there is hope that protein-protein interactions (PPIs) involving MYC can be targeted therapeutically. The MYC DNA-binding domain heterodimerizes with MAX and together they form a tight complex with DNA. Several labs have attempted to find small molecules that inhibit the MYC:MAX interaction with limited success (18-20). One difficulty in targeting this protein-protein interface is that it involves extensive contacts throughout the bHLH and LZ domains, and countless other transcription factors share these motifs (11). Hence, it is very difficult to inhibit MYC:MAX heterodimers without also introducing off-target side effects on other HLH, LZ, or coiled-coil proteins.

### Transformation/Transcription Domain-Associated Protein (TRRAP)

The MYC transactivation domain (TAD) is also involved in several PPIs, including an interaction with the TRansformation/tRanscription domain-Associated Protein (TRRAP). TRRAP has been shown to be a critical MYC cofactor (21-23), and MB2 is required for MYC:TRRAP binding (23, 24). TRRAP is a member of various histone-acetylation (HAT) complexes which aid transcription factors, like MYC, in controlling gene expression. The identification of TRRAP as an essential MYC cofactor established a link to HAT complexes containing GCN5 and TIP60 and provided an important mechanistic insight into MYC's function (17,19,22,23,25). TRRAP is a highly conserved 434 KDa protein that belongs to the Phosphoinositide 3-Kinase-related kinase (PIKK) family that includes mTOR, DNA-PKcs, ATM/Tell, ATR/Mec1 and SM 73 G-1 (26,27). PIKKs are kinases involved in transcriptional regulation, DNA repair, cell growth, metabolic control and mRNA surveillance, but TRRAP lacks a kinase domain and has no enzymatic activity throughout evolution (22,28). Instead, TRRAP is thought to function as a scaffold, bridging transcription factors and chromatin modifying complexes (29, 30). TRRAP is an essential gene, and its disruption leads to early embryonic lethality in mice (25,31). TRRAP mutations have been associated with tumorigenesis, and some models portray TRRAP as an oncogene (32, 33). It is difficult to reconcile the proposed function of TRRAP as a mere scaffold and the observations regarding its role in the cell cycle and disease. Further investigation into the biological functions of TRRAP is warranted.

TRRAP is massive and is involved in various megadalton sized protein complexes. It is a subunit of both the STAGA and NuA4 HAT complexes, which contain GCN5 and Tip60 respectively (19, 25, 34). Although it lacks catalytic activity, TRRAP is critical for transcriptional coactivator function and enables the activities of STAGA and NuA4 to be directed at specific genes in order to stimulate their expression (35). These complexes use TRRAP to mediate their interactions with transcription factors, like MYC, E2F, E1A and p53, making it a conserved activator target in all eukaryotes (23, 36, 37). TRRAP recruitment to DNA results in transcriptional activation by enabling histone modification around gene promoters and hyperacetylation of lysine residues on histone tails (22, 38).

More recently, a Cryo-EM structure of Saccharomyces cerevisiae Tra1p was reported to 3.7 Å resolution revealing the extensive network of α-helical solenoids (40). An atomic model was built with 3474 residues assigned with visible side-chains, but 270 residues were not resolved in the reconstruction. These unresolved residues were distributed across chain breaks that contain either loops or disordered regions. Tra1p was found to have HEAT, FAT, FRB, kinase and FATC domains arranged sequentially from N- to C-terminus, which are characteristic of PIKK family proteins (26, 27). A prediction of TRRAP's secondary structure, aligned with Tra1p, revealed 98% overlap in helical repeats, even though the sequences of the two proteins are only 27% identical (41).

### The MYC:TRRAP Interaction

The MYC:TRRAP interaction has been roughly mapped (23, 39), however, the precise domains of this PPI have not been described. McMahon et al. established that MB2 is required for the MYC:TRRAP interaction (39), but did not describe the minimal MYC domain that is sufficient for TRRAP binding. Similarly, the minimal sufficient MYC-binding domain of TRRAP has not been described. The identification of these minimal domains required for the MYC:TRRAP PPI is important for further studies. As such, further characterization of the MYC:TRRAP interaction is needed. What is more, there is a need to identify and develop small molecules which could therapeutically target MYC in cancer, and in particular, inhibit the MYC:TRRAP interaction. Accordingly, among the objects herein, it is an object herein to provide methods, compounds, and compositions toward that end.

### BRIEF SUMMARY

The present disclosure generally relates to a method for identifying an inhibitor of a binding interaction between MYC transcription factor and Transformation/Transcription Domain-Associated Protein (TRRAP). The method may comprise (a) forming a MYC:TRRAP complex having a MYC:TRRAP binding interaction; (b) directly and/or indirectly detecting the MYC:TRRAP complex and/or the MYC:TRRAP binding interaction to determine a baseline measurement for the MYC:TRRAP complex and/or the MYC:TRRAP binding interaction; (c) introducing a chemical compound prior to or after forming a MYC:TRRAP complex having a MYC:TRRAP binding interaction; and (d) determining an absence or a reduction of the MYC:TRRAP complex and/or the MYC:TRRAP binding interaction after the chemical compound has been introduced compared to the baseline measurement, wherein the absence or the reduction of the MYC:TRRAP complex and/or the MYC:TRRAP binding interaction indicates that the chemical compound is an inhibitor of the binding interaction between MYC and TRRAP.

In some embodiments, MYC may comprise an amino acid sequence which is at least 75% identical, at least 80% identical, at least 85% identical, at least 90% identical, at least 95% identical, at least 98% identical, or at least 99% identical to SEQ ID NO: 2 or to another mammalian MYC amino acid sequence. In some embodiments, TRRAP may comprise an amino acid sequence which is at least 75% identical, at least 80% identical, at least 85% identical, at least 90% identical, at least 95% identical, at least 98% identical, or at least 99% identical to SEQ ID NO: 4 or to another mammalian TRRAP amino acid sequence.

In some embodiments, the MYC:TRRAP complex may be formed in an in vitro environment. In some embodiments, the MYC:TRRAP complex may be formed in a cell. The cell may be selected from a human cell, a mammalian cell, an insect cell, a yeast cell, and a bacterial cell. In some embodiments, the MYC:TRRAP complex may be formed in a non-human animal selected from C. elegans, D. melanogaster, a zebrafish, a rodent, and a non-human primate. In some embodiments, the MYC:TRRAP complex may be formed from endogenous MYC and endogenous TRRAP. In some embodiments, the MYC:TRRAP complex may be formed from endogenous MYC and an exogenous TRRAP or an exogenous TRRAP fragment. In some embodiments, the MYC:TRRAP complex may be formed from endogenous TRRAP and an exogenous MYC or an exogenous MYC fragment. In some embodiments, the MYC:TRRAP complex may be formed from an exogenous MYC or an exogenous MYC fragment and an exogenous TRRAP or an exogenous TRRAP fragment. In some embodiments, the exogenous MYC or the exogenous MYC fragment may be introduced into the cell or may be expressed from an exogenous nucleic acid which may be introduced into the cell or into the non-human animal. In some embodiments, the exogenous TRRAP or the exogenous TRRAP fragment may be introduced into the cell or may be expressed from an exogenous nucleic acid which may be introduced into the cell or into the non-human animal. In some embodiments, the exogenous nucleic acid may be selected from DNA, RNA, mRNA, a plasmid, a vector, and a viral construct. In some embodiments, the cell or the non-human animal may be genetically engineered to express the exogenous MYC, the exogenous MYC fragment, the exogenous TRRAP, and/or the exogenous TRRAP fragment.

In some embodiments, the MYC:TRRAP complex may comprise a full-length MYC and a full-length TRRAP. In some embodiments, the MYC:TRRAP complex may comprise a MYC fragment and a TRRAP fragment. In some embodiments, the MYC:TRRAP complex may comprise a full-length MYC and a TRRAP fragment. In some embodiments, the MYC:TRRAP complex may comprise a MYC fragment and a full-length TRRAP. In some embodiments, the MYC:TRRAP complex may comprise a MYC-TRRAP fusion comprising a full-length MYC, a linker, and a full-length TRRAP. In some embodiments, the MYC:TRRAP complex may comprise a MYC-TRRAP fusion comprising a MYC fragment, a linker, and a TRRAP fragment. In some embodiments, the MYC:TRRAP complex may comprise a MYC-TRRAP fusion comprising a full-length MYC, a linker, and a TRRAP fragment. In some embodiments, the MYC:TRRAP complex may comprise a MYC-TRRAP fusion comprising a MYC fragment, a linker, and a full-length TRRAP. In some embodiments, the MYC fragment may comprise a minimal MYC region. In some embodiments, the minimal MYC region may be a MYC MB2 domain. In some embodiments, the TRRAP fragment may comprise a minimal TRRAP region. In some embodiments, the minimal TRRAP region may be a TRRAP 2033-2088 region. In some embodiments, the linker may comprise the amino acid sequence of SEQ ID NO: 5.

In some embodiments, the full-length MYC may comprise an affinity tag, a detectable label, and/or a distinct protein, protein domain, or protein fragment useful for purification, identification, and/or complementation. In some embodiments, the MYC fragment may comprise an affinity tag, a detectable label, and/or a distinct protein, protein domain, or protein fragment useful for purification, identification, and/or complementation. In some embodiments, the full-length TRRAP may comprise an affinity tag, a detectable label, and/or a distinct protein, protein domain, or protein fragment useful for purification, identification, and/or complementation. In some embodiments, the TRRAP fragment may comprise an affinity tag, a detectable label, and/or a distinct protein, protein domain, or protein fragment useful for purification, identification, and/or complementation.

In some embodiments, the MYC fragment may be a MYC 129-145 fragment (i.e., a MYC MB2 fragment or MB2 domain). In some embodiments, the MYC 129-145 fragment may comprise an amino acid sequence which is at least 75% identical, at least 80% identical, at least 85% identical, at least 90% identical, at least 95% identical, at least 98% identical, or at least 99% identical to SEQ ID NO: 6 or to a corresponding MYC 129-145 amino acid sequence from a non-human mammalian species obtained by aligning a MYC amino acid sequence from one or more non-human mammalian species with SEQ ID NO: 2 and selecting the amino acid residues which align with amino acid residues 129-145 of SEQ ID NO: 2.

In some embodiments, the MYC fragment may be a MYC 1-190 fragment. In some embodiments, the MYC 1-190 fragment may comprise an amino acid sequence which is at least 75% identical, at least 80% identical, at least 85% identical, at least 90% identical, at least 95% identical, at least 98% identical, or at least 99% identical to SEQ ID NO: 7 or to a corresponding MYC 1-190 amino acid sequence from a non-human mammalian species obtained by aligning a MYC amino acid sequence from one or more non-human mammalian species with SEQ ID NO: 2 and selecting the amino acid residues which align with amino acid residues 1-190 of SEQ ID NO: 2.

In some embodiments, the MYC fragment may be a MYC 120-161 fragment. In some embodiments, the MYC 120-161 fragment may comprise an amino acid sequence which is at least 75% identical, at least 80% identical, at least 85% identical, at least 90% identical, at least 95% identical, at least 98% identical, or at least 99% identical to SEQ ID NO: 8 or to a corresponding MYC 120-161 amino acid sequence from a non-human mammalian species obtained by aligning a MYC amino acid sequence from one or more non-human mammalian species with SEQ ID NO: 2 and selecting the amino acid residues which align with amino acid residues 120-161 of SEQ ID NO: 2.

In some embodiments, the TRRAP fragment may be a TRRAP 2033-2088 fragment. In some embodiments, the TRRAP 2033-2088 fragment may comprise an amino acid sequence which is at least 75% identical, at least 80% identical, at least 85% identical, at least 90% identical, at least 95% identical, at least 98% identical, or at least 99% identical to SEQ ID NO: 9 or to a corresponding TRRAP 2033-2088 amino acid sequence from a non-human mammalian species obtained by aligning a TRRAP amino acid sequence from one or more non-human mammalian species with SEQ ID NO: 4 and selecting the amino acid residues which align with amino acid residues 2033-2088 of SEQ ID NO: 4.

In some embodiments, the TRRAP fragment may be a TRRAP 2033-2283 fragment. In some embodiments, the TRRAP 2033-2283 fragment may comprise an amino acid sequence which is at least 75% identical, at least 80% identical, at least 85% identical, at least 90% identical, at least 95% identical, at least 98% identical, or at least 99% identical to SEQ ID NO: 10 or to a corresponding TRRAP 2033-2283 amino acid sequence from a non-human mammalian species obtained by aligning a TRRAP amino acid sequence from one or more non-human mammalian species with SEQ ID NO: 4 and selecting the amino acid residues which align with amino acid residues 2033-2283 of SEQ ID NO: 4.

In some embodiments, the chemical compound may be an isolated chemical compound. In some embodiments, the chemical compound may be comprised in a mixture of chemical compounds. In some embodiments, the chemical compound may comprise a small-molecule organic chemical compound. In some embodiments, the chemical compound may be selected from a small-molecule chemical compound library. In some embodiments, the chemical compound may be introduced at various concentrations ranging from 10 nM to 100 μM. In some embodiments, the method may further comprise determining an IC50 value for the chemical compound. In some embodiments, the chemical compound may be introduced at a concentration of 25 μM. In some embodiments, the chemical compound may be selected from a chemical compound listed in Table 1. In some embodiments, the method may further comprise designing, synthesizing, and testing a chemical compound derived from one or more of the chemical compounds listed in Table 1 for an ability of the chemical compound to inhibit the binding interaction between MYC and TRRAP.

In some embodiments, the method may further comprise determining the specificity of the chemical compound for inhibiting the binding interaction between MYC and TRRAP by testing an ability of the chemical compound to inhibit a binding interaction between MYC and the MYC-associated factor MAX.

In some embodiments, the method may further comprise a cell-based protein-fragment complementation assay to detect the MYC:TRRAP complex and/or the MYC:TRRAP binding interaction.

In some embodiments, the cell-based protein-fragment complementation assay may be a luminescence complementation assay. In some embodiments, the luminescence complementation assay may comprise: a. an SmB-luciferase-MYC fusion comprising an N-terminal SmB-luciferase fragment and a C-terminal full-length MYC or a C-terminal MYC fragment; and b. a TRRAP-LgB-luciferase fusion comprising an N-terminal TRRAP fragment and a C-terminal LgB-luciferase fragment; wherein the SmB-luciferase-MYC fusion and the TRRAP-LgB-luciferase fusion form the MYC:TRRAP complex, whereby the SmB-luciferase fragment and the LgB-luciferase fragment form a functional luciferase enzyme which generates a luminescence signal in the presence of a luciferase substrate. In some embodiments, the MYC fragment may be a MYC 1-190 fragment and the TRRAP fragment may be a TRRAP 2033-2283 fragment. In some embodiments, the functional luciferase enzyme may be a 19.1 kDa luciferase enzyme derived from Oplophorus gracilirostris. In some embodiments, the SmB-luciferase-MYC fusion and the TRRAP-LgB-luciferase fusion may each be expressed in the cell from a mammalian expression vector comprising a constitutive promoter. In some embodiments, the promoter may be a CMV promoter. In some embodiments, the expression level of the SmB-luciferase-MYC fusion and the expression level of the TRRAP-LgB-luciferase fusion may be substantially equal. In some embodiments, the cell may be a HeLa cell or an Expi 293 cell or Expi 293 cell suspension. In some embodiments, the luciferase substrate may be furimazine. In some embodiments, the luminescence complementation assay may further comprise detecting a false positive result caused by direct inhibition of the luciferase activity or by inhibition of the complementation of the SmB-luciferase and LgB-luciferase fragments. In some embodiments, the cell may further express a fluorescence reporter, wherein the fluorescence report may be used to normalize transfection efficiency and cell number. In some embodiments, the fluorescence reporter may be EGFP. In some embodiments, the chemical compound may be introduced at various concentrations ranging from 10 nM to 100 μM. In some embodiments, the method may further comprise determining an IC50 value for the chemical compound. In some embodiments, the chemical compound may be introduced at a concentration of 25 μM and may reduce the luminescence signal by at least 50%. In some embodiments, the chemical compound may be selected from a chemical compound listed in Table 1, Table 2, Table 5 or may comprise one of the 4 generic structures set forth in Table 4. In some embodiments, the method may further comprise designing, synthesizing, and testing a chemical compound derived from one or more of the chemical compounds listed in Table 1, Table 2, Table 5 or one comprising one of the 4 generic structures set forth in Table 4 for the ability of the chemical compound to inhibit the binding interaction between MYC and TRRAP.

In some embodiments, the method may further comprise co-purification of the MYC:TRRAP complex from cells to detect the MYC:TRRAP complex and/or the MYC:TRRAP binding interaction. The cells may be selected from human cells, mammalian cells, insect cells, yeast cells, and bacterial cells.

In some embodiments, the method may further comprise co-immunoprecipitation of the MYC:TRRAP complex from a cell lysate to detect the MYC:TRRAP complex and/or the MYC:TRRAP binding interaction. The cell lysate may be selected from a human cell lysate, a mammalian cell lysate, an insect cell lysate, a yeast cell lysate, and a bacterial cell lysate. In some embodiments, the co-immunoprecipitation may comprise the full-length MYC or MYC fragment having a first affinity tag and the full-length TRRAP or TRRAP fragment having a second affinity tag and, wherein: a. the full-length MYC or MYC fragment having a first affinity tag and the full-length TRRAP or TRRAP fragment having a second affinity tag are co-expressed in the cell; and b. the first affinity tag and the second affinity tag are different. In some embodiments, the co-immunoprecipitation may comprise: a. the full-length MYC or MYC fragment having a first affinity tag wherein the full-length MYC or MYC fragment having a first affinity tag is expressed in the cell and co-immunoprecipitates endogenous TRRAP; or b. the full-length TRRAP or TRRAP fragment having a first affinity tag; wherein the full-length TRRAP or TRRAP fragment having a first affinity tag is expressed in the cell and co-immunoprecipitates endogenous MYC. In some embodiments, the first affinity tag and the second affinity tag may be selected from a PYO tag and a FLAG tag, optionally wherein the first affinity tag and the second affinity tag are different. In some embodiments, the MYC:TRRAP complex may be detected by Western Blot analysis using an anti-MYC antibody, an anti-TRRAP antibody, an anti-FLAG antibody, and/or an anti-PYO antibody. In some embodiments, the MYC fragment may be a MYC 1-190 fragment and the TRRAP fragment may be a TRRAP 2033-2283 fragment. In some embodiments, the cell lysate may be a human cell lysate. In some embodiments, the cell lysate may be a HEK293T cell lysate.

In some embodiments, the protein-stabilizing additive may be selected from ethylene glycol (EG), 2,2,2-trifluoroethanol (TFE), and deuterated TFE (TFE-d2), or any combination of these. In some embodiments, the protein-stabilizing additive may comprise a concentration ranging from about 5% (v/v) to about 50% (v/v) in the in vitro environment. In some embodiments, the protein-stabilizing additive may comprise a concentration ranging from about 20% (v/v) to about 30% (v/v) in the in vitro environment.

In some embodiments, the method may further comprise an in vitro pulldown assay to detect the MYC:TRRAP complex and/or the MYC:TRRAP binding interaction. In some embodiments, the in vitro pulldown assay may comprise the MYC:TRRAP complex formed from the MYC-TRRAP fusion, wherein the MYC-TRRAP fusion comprises at least one affinity tag. In some embodiments, the MYC-TRRAP fusion may comprise a MYC 1-190 fragment, a linker, a TRRAP 2033-2088 fragment, and an affinity tag. In some embodiments, the method may further comprise proteolytic cleavage of the MYC:TRRAP fusion at a protease cleavage site within the linker. The protease cleavage site may be any unique protease cleavage site within the MYC:TRRAP fusion. In some embodiments, the protease cleavage site may be a 3C protease cleavage site or a TEV cleavage site.

In some embodiments, the method may further comprise a nuclear magnetic resonance (NMR) assay to detect the MYC:TRRAP complex and/or the MYC:TRRAP binding interaction. In some embodiments, the NMR assay may comprise: a. the MYC:TRRAP complex formed from the MYC-TRRAP fusion; b. 1H, 15N-HSQC NMR; and c. one or more chemical shift peaks indicative of a chemical environment of MYC W135; wherein the one or more chemical shift peaks are different when the MYC:TRRAP binding interaction is present compared to when the MYC:TRRAP binding interaction is absent. In some embodiments, the MYC-TRRAP fusion may comprise a MYC 120-161 fragment, a linker, and a TRRAP 2033-2088 fragment.

In some embodiments, the method may further comprise intrinsic fluorescence of MYC W135 to detect the MYC:TRRAP complex and/or the MYC:TRRAP binding interaction, wherein the intrinsic fluorescence of MYC W135 is different when the MYC:TRRAP binding interaction is present compared to when the MYC:TRRAP binding interaction is absent.

In some embodiments, the method may further comprise in silico computational analysis of the MYC:TRRAP complex and/or the MYC:TRRAP binding interaction.

In some embodiments, the cell-based protein-fragment complementation assay may be a biomolecular fluorescence complementation (BiFC) assay.

In some embodiments, the method may further comprise size exclusion chromatography to detect the MYC:TRRAP complex and/or the MYC:TRRAP binding interaction.

In some embodiments, the method may further comprise bioluminescence resonance energy transfer (BRET) to detect the MYC:TRRAP complex and/or the MYC:TRRAP binding interaction.

In some embodiments, the method may further comprise fluorescence resonance energy transfer (FRET) to detect the MYC:TRRAP complex and/or the MYC:TRRAP binding interaction.

In some embodiments, the method may further comprise fluorescence polarization (FP) and/or fluorescence anisotropy (FA) to detect the MYC:TRRAP complex and/or the MYC:TRRAP binding interaction.

In some embodiments, the method may further comprise surface plasmon resonance (SPR) to detect the MYC:TRRAP complex and/or the MYC:TRRAP binding interaction.

In some embodiments, the method may further comprise native polyacrylamide gel electrophoresis (PAGE) to detect the MYC:TRRAP complex and/or the MYC:TRRAP binding interaction.

In some embodiments, the method may further comprise a protein microarray to detect the MYC:TRRAP complex and/or the MYC:TRRAP binding interaction.

In some embodiments, the method may further comprise a microfluidic assay to detect the MYC:TRRAP complex and/or the MYC:TRRAP binding interaction.

In some embodiments, the method may further comprise electron microscopy to detect the MYC:TRRAP complex and/or the MYC:TRRAP binding interaction.

Furthermore, the present disclosure generally relates to a method for developing a cancer therapeutic comprising: a. identifying an inhibitor of a binding interaction between MYC and TRRAP by any of the methods described herein; b. optionally derivatizing the identified inhibitor to produce a derivatized inhibitor and testing the derivatized inhibitor for an ability to inhibit a binding interaction between MYC and TRRAP; and c. testing the inhibitor or the derivatized inhibitor for an ability to treat cancer in a subject.

Moreover, the present disclosure generally pertains to a method for treating a subject having at least one cancer comprising, administering a therapeutically effect amount of a chemical compound to the subject, wherein the chemical compound has been identified to be an inhibitor of a binding interaction between MYC and TRRAP by any of the methods described herein.

In some embodiments, the subject may be a mammal selected from a rodent, a non-human primate, and a human. In some embodiments, the subject may be a human. In some embodiments, the at least one cancer may be selected from one or more of adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures.

Moreover, the present disclosure generally relates to a chemical compound for use as an inhibitor of a binding interaction between MYC and TRRAP, wherein the chemical compound is selected from a chemical compound listed in Table 1. In some embodiments, the chemical compound may be:

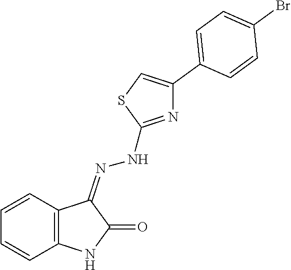 [SMILES: O=C1Nc2ccccc2/C1=N/Nc1nc(-c2ccc(Br)cc2)cs1]

or a derivative thereof.

In some embodiments, the chemical compound may be a derivative of a chemical compound listed in Table 1, Table 2, Table 5 or one comprising one of the 4 generic structures set forth in Table 4.

Furthermore, the present disclosure generally relates to a composition comprising a chemical compound as described herein and a pharmaceutically suitable carrier. In some embodiments, the composition may comprise a derivative of a chemical compound as described herein and a pharmaceutically suitable carrier.

Moreover, the present disclosure generally relates to a method for treating a subject having at least one cancer comprising administering a therapeutically effective amount of a chemical compound as described herein. In some embodiments, the method for treating a subject having at least one cancer may comprise administering a therapeutically effective amount of a derivative of a chemical compound as described herein.

In some embodiments, the subject may be a mammal selected from a rodent, a non-human primate, and a human. In some embodiments, the subject may be a human. In some embodiments, the at least one cancer may be selected from one or more of adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures.

### DESCRIPTION OF THE DRAWINGS

FIG. 1A-FIG. 1C present data regarding the minimal interacting domains of MYC:TRRAP. (A) The 231 indicated regions of TRRAP were cloned into a CMV-FLAG expression vector. Proteins were co-expressed with PYO-tagged full-length MYC (1-439) and then MYC was IPed with anti-PYO beads. Co-IP was assessed by western blot with anti-FLAG. The most critical binding domain is within residues 1997-2088. (B) Full-length TRRAP (1-3830) and TRRAP Δ2033-2088 were cloned into a CMV-FLAG expression vector and transfected into HEK293T cells. Proteins were co-expressed with PYO237 tagged full-length MYC and then MYC was IPed with anti-PYO beads. Co-IP was evaluated by western blot. TRRAP Δ2033-2088 shows reduced binding to MYC. (C) Full-length MYC, MYC ΔMB2, MYC 1-190, and MYC 1-190 ΔMB2 were cloned into a CMV-PYO expression vector and transfected into HEK293T cells. Proteins were co-expressed with FLAG-tagged TRRAP 2033-2283 then MYC was IPed with anti-PYO beads. Co-IP was evaluated by western blot. TRRAP 2033-2283 shows equal co-IP with full-length MYC as with MYC 1-190, and both require MB2.

FIG. 2A-FIG. 2B present data regarding endogenous co-IP confirmation. (A) MYC 1-190 and MYC 1-190 ΔMB2 were cloned into a CMV-PYO expression vector and transfected into HEK293T cells, then MYC was IPed with anti-PYO beads. Co-IP of endogenous TRRAP was evaluated by western blot. Endogenous TRRAP can co-IP with MYC 1-190 but requires MB2. (B) MYC, MYC ΔMB2, and MYC W135G were cloned into a CMV-PYO expression vector and transfected into HEK293T cells, then MYC was IPed with anti-PYO beads. Co-IP of endogenous TRRAP was evaluated by western blot. Endogenous TRRAP can co-IP with MYC and requires MB2 and W135.

FIG. 3A-FIG. 3B present data regarding protein purification strategy. (A) The general protein purification strategy involved the production of a protein construct in E. coli expressed by a modified pGEX vector containing both an N-terminal GST tag and a C-terminal TS tag. (B) A Coomassie-stained SDS-PAGE after production and lysis, cleared lysates were subjected to a glutathione column and the protein construct was eluted. It was then loaded on a StrepTactin® XT column and eluted a second time with biotin. The eluate was then subjected to a cleavage reaction by TEV protease carried out at 4° C. for 16h. Next, both the GST tag and TEV protease were removed on agarose glutathione beads. The TS tag was subsequently removed on StrepTactin® XT beads. Finally, the sample was loaded on an SEC column. After this final purification step, it was concentrated, flash frozen, and stored at −80° C.

FIG. 4A-FIG. 4C present data regarding a MYC:TRRAP complex does not form in vitro. (A) CD spectra of MYC 1-190 mixed in vitro with TRRAP 2033-2088 at 10 μM each. (B) CD spectra of MYC 1-190 ΔMB2 mixed in vitro with TRRAP 2033-2088 at 10 μM each. CD spectra show no gain in secondary structure after mixing either MYC 1-190 or MYC 1-190 ΔMB2 with TRRAP 2033-2088. (C) Coomassie-stained SDS-PAGE of a TS tag pulldown of TRRAP 2033-2088 mixed with MYC 1-190 and MYC 1-190 ΔMB2 at 50 μM each. This result demonstrates that MYC 1-190 and TRRAP 2033-2088 do not interact when mixed in vitro.

FIG. 5A-FIG. 5K present data regarding the effects of additives on MYC and TRRAP. (A-K) CD spectra of MYC 1-190, MYC 1-190 mixed with TRRAP 2033-2088, and TRRAP 2033-2088 with the indicated additives at the indicated concentration.

FIG. 6A-FIG. 6D present data regarding effects of ethylene glycol on MYC and TRRAP. (A-C) CD spectra of MYC 1-190, TRRAP 2033-2088, and BSA. Solid lines represent measurements taken in 1×PBS; dotted lines represent measurements taken in 30% EG. A significant increase in the α-helical character of MYC and (to a lesser extent) TRRAP is observed in the presence of EG. However, BSA (a highly α-helical well-folded protein) appears unaffected by the presence of EG. (D) SEC A280 spectra of MYC 1-190 (black), TRRAP 2033-2088 (grey), and MYC 1-190 mixed with TRRAP 2033-2088 (black dotted) in 30% EG all at 100 μM. Neither MYC nor TRRAP showed any variation in their expected hydrodynamic radius as measured in 1×PBS. The mixed sample did not have any measurable tertiary peak that would indicate an association between the MYC and TRRAP.

FIG. 7A-FIG. 7B present data regarding the effect of EG on MYC-TRRAP. (A) CD spectra of two MYC-TRRAP fusion proteins in 30% EG: MYC 1-190-TRRAP 2033-2088 in black and MYC 1-190 ΔMB2-TRRAP 2033-2088 in red. The effects of EG on the fusion protein containing MB2 are more profound and are indicative of a specific gain in α-helical character. (B) Coomassie-stained SDS-PAGE of a pulldown of two 3C protease-cleavable fusion proteins. MYC 1-190-TRRAP 2033-2088-TS and MYC 1-190 ΔMB2-TRRAP2033-2088-TS incubated in either 1×PBS or 30% EG. After 3C cleavage of the linker, the TRRAP domain was pulled down with StrepTactin® beads and the EG was washed away with 1×PBS. MYC 1-190 showed enhanced binding to TRRAP 2033-2088 in the presence of 30% EG but not in PBS, when compared to MYC 1-190 ΔMB2 in 30% EG.

FIG. 8A-FIG. 8D present data regarding endogenous co-IP confirmation. (A) CD spectra of 710 MYC 1-190, MYC 1-190 ΔMB2, MYC 120-161, and TRRAP 2033-2088 demonstrate that all four are intrinsically disordered. The lack of significant minima at wavelengths 208 nm, 215 nm, and 222 nm suggest that these constructs lack ordered secondary structure. This is confirmed also by the overall shapes of the curves with minima at 202 nm. However, the slight minima observed at 222 nm in MYC 1-190 and MYC 120-161 suggest that there might be some α-helical structural elements present. (B) CD spectra of MYC 120-161-TRRAP 2033-2088 in 0%-90% (v/v) TFE. Increasing TFE concentration is indicated by increasing darkness in color. TFE induces a gain in α-helical secondary structure with each increase in concentration. (C, D) 1H-NMR spectra of MYC 120-161 in 1×PBS (left) and 30% TFE-d2 (right). Bottom panels are enlarged from 6-10 ppm of the above spectra. The spectrum of MYC 120-161 in PBS indicates the presence of significant unstructured elements based on the large cluster of severely overlapped peaks. However, in the presence of TFE, the peaks become well-dispersed and individual peaks can be distinguished, which indicates a well-folded protein.

FIG. 9A-FIG. 9B present data regarding the environment of W135 in MYC 120-161 vs MYC 120-161-TRRAP 2033-2088. (A) 1H, 15N-HSQC spectra of MYC 120-161 in 30% TFE-d2. (B) 1H, 15N-HSQC spectra of MYC 120-161-TRRAP 2033-2088 in 30% TFE-d2. The peak shifts of W135 in the MYC-TRRAP fusion spectrum is indicative of a binding event. The splitting of the peak suggests two stable conformations: bound and unbound states.

FIG. 10A-FIG. 10B present data regarding combinations of MYC and TRRAP pairs for luminescence complementation assay. (A) Four constructs each were created using MYC 1-190 and TRRAP 2033-2283 with NanoBiT tags, LgB and SmB. All possible eight combinations that can result in luminescence complementation are shown. Each of these combinations was transfected into HeLa cells and luminescence was measured to determine which pair had the best signal-to-noise ratio. (B) Pairs of full-length MYC with TRRAP 2033-2283 (top) and MYC 1-190 and TRRAP 2033-2283 (bottom) that gave the best signal-to-noise luminescence.

FIG. 11 presents data regarding MYC's dependence on MB2 in cells. Luminescence measurements of HeLa cells transfected with the indicated MYC and TRRAP 2033-2283 pairs or with LgB in excess. The same amount of DNA was used for each MYC construct. The graph shows MYC's dependence on MB2 for TRRAP binding and equal expression of MYC and MYC ΔMB2.

FIG. 12 presents data regarding MYC 1-190's dependence on MB2 in cells. Luminescence measurements of HeLa cells transfected with the indicated MYC 1-190 and TRRAP 2033-2283 pairs or with LgB in excess. The same amount of DNA was used for each MYC 1-190 construct. The graph shows MYC 1-190's dependence on MB2 for TRRAP binding and higher expression of MYC 1-190 ΔMB2 compared to MYC 1-190.

FIG. 13 presents data regarding normalized MYC 1-190's dependence on MB2 in cells. Luminescence measurements of HeLa cells transfected with the indicated MYC 1-190 and TRRAP 2033-2283 pairs or with LgB in excess. Seven times more DNA was transfected of MYC 1-190 than MYC 1-190 ΔMB2 construct. The graph shows MYC 1-190's dependence on MB2 for TRRAP binding and equal expression of MYC 1-190 and MYC 1-190 ΔMB2.

FIG. 14 presents data regarding TRRAP's dependence on TRRAP 2033-2088 in cells. Luminescence measurements of HeLa cells transfected with the indicated MYC 1-190 and TRRAP 2033-2283 pairs or with SmB in excess. Nine times more DNA was transfected for each TRRAP construct compared to the MYC construct. The graph shows TRRAP 2033-2283's dependence on 2033-2088 for MYC binding and equal expression of TRRAP 2033-2283 and TRRAP 2088-2283.

FIG. 15 presents data regarding MYC substitution mutations' effect on TRRAP binding. Luminescence measurements of HeLa cells transfected with TRRAP 2033-2283 and the indicated MYC 1-190 or mutant pairs. Seven times more DNA was transfected of MYC 1-190 and all other mutants except for MYC 1-190 ΔMB2. The graph confirms MYC 1-190's dependence on W135 for TRRAP binding and shows the effects of other point mutations on the interaction.

FIG. 16 presents data regarding small-molecule inhibitors of MYC:TRRAP in the luminescence complementation assay. Structures of Compounds 1-25 are shown in Table 1.

FIG. 17 presents data regarding inhibitors' effects on endogenous MYC and TRRAP. Western blot analysis of the effects of compounds 1-17 from FIG. 16 on the indicated endogenous proteins. HeLa cells were incubated in the presence of each of the indicated compounds at 25 μM for 2 h prior to analysis.

FIG. 18A-FIG. 18H present data regarding NC160 G150 correlations with MYC expression of exemplary compounds from FIG. 16.

FIG. 19 presents data regarding inhibitors' effects on endogenous MYC:TRRAP Co-IP. Western blot analysis of co-IP experiments determining the effects of the compounds from FIG. 16 on the indicated endogenous complexes. HeLa cells were incubated in the presence of each of the indicated compounds at 25 μM for 2 h prior to analysis.

FIG. 20 presents data regarding quantification of inhibition of MYC:TRRAP Co-IP. LI-COR Odyssey® laser density quantification of the images presented in FIG. 19.

FIG. 21 presents a heat map summarizing the compounds from FIG. 16 that showed co-IP inhibition of the endogenous MYC:TRRAP complex.

FIG. 22A-FIG. 22E present data evidencing the dependency of MYC:TRRAP inhibitors on concentration. (A-E) MYC:TRRAP in-cell luminescence complementation inhibition measurements with incubation at varying concentration of the indicated compounds.

FIGS. 23A-23B show the results of co-IP assay experiments comparing the top 17 hits in immunoprecipitation experiments using endogenous full length MYC and TRRAP. In these experiments immunocomplexes were immunoprecipitated with anti-MYC beads in the presence of 25 μM of each of the 17 top compounds. Experiments were carried out in cells (compounds were added to the media of cells) (FIG. 23B) or in vitro (compounds were added directly to the purified complex on pre-washed beads) (FIG. 23A). Immunoprecipitation of TRRAP was normalized to the immunoprecipitation of MYC. As shown therein Compound 10 (NSC657456) exhibited the greatest inhibitory activity.

FIGS. 24A-24C show the results of an experiment comparing the effects of compounds similar in structure to Compound 10 on MYC:TRRAP inhibitory activity which showed that even small changes in the chemical structure of the lead compound can disrupt or eliminate MYC:TRRAP inhibitory activity. In these experiments compounds possessing similar structures to Compound 10 (NSC657456) were screened with an luminescence assay and co-IP assay as described infra. Measurements were compared to a DMSO vehicle control. As shown therein compound NSC657456 (FIG. 24B) inhibited MYC:TRRAP complex formation by 70% whereas a structurally similar compound NSC657457 (FIG. 24C) only inhibited MYC:TRRAP complex formation by 20% (FIG. 24A).

FIGS. 25A-25M show the results of experiments which showed that similarity screening increases MYC:TRRAP inhibitory activity. Luminescence measurements of HeLa cells transfected with SmB-MYC 1-190 and TRRAP 2033-2283-LgB and incubated with each of the indicated compounds (FIGS. 25D-25M) at the indicated concentrations were conducted (FIGS. 25A-25C). The original compound 10 is NSC657456 (FIG. 23, 24). This compound set was designed with >80% similarity to NSC657456.

FIGS. 26A-26B show the results of experiments which demonstrate that NSC657587 potently inhibits MYC:TRRAP complexes and wherein measurements were normalized to protein levels for both MYC and TRRAP. As shown therein NSC657587 had the lowest IC50 for the inhibition of MYC:TRRAP, both in measurements in cells with the in-cell luminescence complementation assay (4.7 μM; top; FIG. 26A) and in vitro by co-IP (3.7 μM; bottom; FIG. 26B). This represents an ^{~}10-fold increase in activity from NSC657456.

FIGS. 27A-27C show a results of a transfection protocol using Expi293 cells (obtained from ThermoFisher) which shows that these cells elicit about 100-fold more luminescent signal than HeLa cells while maintaining the same signal to noise ratio.

### DETAILED DESCRIPTION

### 1. Overview

Provided are methods and compositions for identifying inhibitors of an interaction between the oncogenic transcription factor MYC and its cofactor TRRAP. In general, the method involves forming a MYC:TRRAP complex having a MYC:TRRAP binding interaction, directly and/or indirectly detecting the MYC:TRRAP complex and/or the MYC:TRRAP binding interaction to determine a baseline measurement for the MYC:TRRAP complex and/or the MYC:TRRAP binding interaction, introducing a chemical compound prior to or after forming a MYC:TRRAP complex having a MYC:TRRAP binding interaction, and determining an absence or a reduction of the MYC:TRRAP complex and/or the MYC:TRRAP binding interaction after the chemical compound has been introduced compared to the baseline measurement, wherein the absence or the reduction of the MYC:TRRAP complex and/or the MYC:TRRAP binding interaction indicates that the chemical compound is an inhibitor of the binding interaction between MYC and TRRAP.

The present disclosure specifically contemplates several approaches whereby chemical compounds may be screened and tested for an ability to inhibit an interaction between MYC and TRRAP. The methods involve both cell-based and in vitro approaches for forming and detecting an interaction between MYC and TRRAP and for identifying inhibitors of a MYC-TRRAP interaction.

The cell-based methods may include a protein-fragment complementation assay, such as a luminescence complementation assay. The cell may be selected from a human cell, a mammalian cell, an insect cell, a yeast cell, and a bacterial cell. The cell-based methods may also include cells within a non-human animal selected from C. elegans, D. melanogaster, a zebrafish, a rodent, and a non-human primate.

The cell-based methods may also include cell-based and in vitro steps, such as co-purification of endogenous MYC and TRRAP from cell lysate. The cell-based methods may include cellular co-expression and co-purification of exogenous MYC and TRRAP, MYC and TRRAP fragments, or a MYC-TRRAP fusion from cell lysate. The cell-based methods may include cellular co-expression and co-immunoprecipitation of tagged MYC and TRRAP from cell lysate.

The in vitro approaches may include formation and detection of a MYC:TRRAP complex in any in vitro environment and may comprise any protein-protein interaction assay known in the art. For example, the in vitro methods may include a pulldown assay, an NMR assay, an intrinsic fluorescence assay, a biomolecular fluorescence complementation (BiFC) assay, size exclusion chromatography, a bioluminescence resonance energy transfer (BRET) assay, a fluorescence resonance energy transfer (FRET) assay, a fluorescence polarization (FP) and/or fluorescence anisotropy (FA) assay, surface plasmon resonance (SPR), native polyacrylamide gel electrophoresis (PAGE), a protein microarray, a microfluidic assay, and electron microscopy.

The in vitro methods may further comprise a MYC-TRRAP fusion having a linker with a protease cleavage site, such as a 3C protease cleavage site. The in vitro methods may also include a protein-stabilizing additive, such as ethylene glycol (EG), 2,2,2-trifluoroethanol (TFE), and deuterated TFE (TFE-d2), or any combination of these. The identity and concentration of protein-stabilizing additive may be determined using circular dichroism. For example, protein-stabilizing additive may have a concentration ranging from about 5% (v/v) to about 50% (v/v), or from about 20% (v/v) to about 30% (v/v).

It is also contemplated that the methods may involve in silico computational analysis of the MYC:TRRAP complex and in silico screening of chemical compounds for an ability to disrupt the MYC:TRRAP complex.

Also provided are compounds for use as inhibitors of the MYC/TRRAP interaction, and methods for developing a cancer therapeutic from such compounds, including methods for derivatizing such inhibitors and for testing the inhibitors and derivatized inhibitors for an ability to treat cancer in a subject. The methods, compounds, and compositions provided herein can provide various advantages, such as a means to target the oncogenic transcription factor MYC in cancer.

Carcinogenesis originates at the cellular level. Complex and interconnected signaling networks govern cellular processes, like growth and proliferation, and respond to both external and internal stimuli. These signaling pathways are hijacked by cancer cells and deregulated to confer proliferative advantages. Cancer cells evolve through a multistage process, driven by the progressive accumulation of multiple genetic and epigenetic abnormalities. Despite the complexity of carcinogenesis, the process is fragile: the growth and survival of cancerous cells can be impaired by the inactivation of a single oncogene (1). Altered transcriptional programs can also make cancer cells highly dependent on certain regulators of gene expression (2). Therefore, research into mechanisms of cellular proliferation carries the promise of discovering new therapies. Extensive studies sequencing the genome of tumors have revealed recurrent somatic mutations that affect normal transcriptional control (2). One of these, a master regulator of transcription, is MYC. It plays a central role in carcinogenesis and is an attractive target for a new generation of drugs that perturb dysregulated transcriptional programs. The fact that many cancer cells cannot survive without MYC—a phenomenon termed “MYC addiction”—provides a compelling case for the development of MYC-specific targeted therapies as disclosed herein.

Exploiting cancer dependencies for medicinal purposes has already led to the development of mechanism-based targeted therapies. Rather than interfering with all rapidly dividing cells (chemotherapy), targeted therapy specifically blocks the growth of cancer cells by interfering with pathways needed for carcinogenesis. Numerous studies have shown that MYC is unique and essential for tumorigenesis and disease progression, and therefore, a good candidate for targeted inhibition (1). TRRAP is a MYC MB2 cofactor and therefore therapeutically targeting MB2 will involve its interaction with TRRAP as disclosed herein.

While human MYC is composed of 439 amino acid resides, TRRAP is much larger (3859 residues). The identification of their respective binding regions and minimal interacting domains disclosed herein has greatly facilitated the study of their interaction. As disclosed herein, MYC 1-190 and TRRAP 2033-2283 display similar interaction characteristics as their full-length counterparts, measured by co-IP or in-cell PPI luminescence complementation. These small MYC and TRRAP constructs have enabled structural studies of the interaction between MYC and TRRAP and development of a method for identifying inhibitors, such as small-molecule inhibitors, of said interaction, as disclosed herein.

It will be understood that particular embodiments described herein are shown by way of illustration and not as limitations. The principal features of this disclosure can be employed in various embodiments without departing from the scope of the disclosure. Those skilled in the art will recognize, or be able to ascertain, using no more than routine experimentation, numerous equivalents to the specific procedures described herein. Such equivalents are considered to be within the scope of this disclosure and are covered by the appended claims.

All publications and patent applications mentioned in the instant specification are indicative of the level of skill of one skilled in the art to which this disclosure pertains. All publications and patent applications are herein incorporated by reference to the same extent as if each individual publication or patent application was specifically and individually indicated to be incorporated by reference.

Unless defined otherwise, all technical and scientific terms used herein have the same meaning as is commonly understood by one of skill in the art to which this disclosure belongs. In the event that there are a plurality of definitions for terms herein, those in this section prevail. Where reference is made to a URL or other such identifier or address, it is to be understood that such identifiers can change and particular information on the internet can come and go, but equivalent information can be found by searching the internet. Reference thereto evidences the availability and public dissemination of such information.

As used herein, the singular forms “a,” “an,” and “the” may mean “one” but also include plural referents such as “one or more” and “at least one” unless the context clearly dictates otherwise. All technical and scientific terms used herein have the same meaning as commonly understood to one of ordinary skill in the art to which this invention belongs unless clearly indicated otherwise.

As used herein, the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 8, 9, 10, 11, 12, 13, 14, or 15%.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply necessarily complete curing of a disease or complete elimination of any symptom or effect(s) on all symptoms or outcomes.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result alone or in combination with other active agents.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts alone or in combination with other active agents or therapies, e.g., those used in cancer treatment.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

As used herein “Expi293” or “Expi293F” cells refer to cells derived from the 293 cell line, which are a core component of the Expi293 Expression System® (ThermoFisher Scientific). They cells are maintained in suspension culture and will grow to high density in Expi293 Expression Medium®. Expi293F cells are highly transfectable and generate superior protein yields compared to standard 293 cell lines in transient protein expression. These cells are also available from a cGMP bank (Cat. No. 100044202).

As used herein, “MYC” and other forms thereof (including “Myc” and “myc”) refers to the MYC transcription factor protein, transcript (mRNA), and/or gene expressing said protein from human (NCBI GeneID No. 4609) or from any other mammalian species, including all isoforms and allelic variants thereof. MYC is also known as MRTL, MYCC, bHLHe39, and c-MYC. MYC may have a cDNA nucleotide sequence which is at least 75% identical, at least 80% identical, at least 85% identical, at least 90% identical, at least 95% identical, at least 98% identical, at least 99% identical or more to SEQ ID NO: 1 or to any other mammalian MYC cDNA sequence. MYC may have an amino sequence which is at least 75% identical, at least 80% identical, at least 85% identical, at least 90% identical, at least 95% identical, at least 98% identical, at least 99% identical or more to SEQ ID NO: 2 or to any other mammalian MYC amino acid sequence. MYC may be expressed on its own or may be expressed as a fusion with a TRRAP or a TRRAP fragment, with a MAX or a MAX fragment, with an affinity tag, with a detectable label, and/or with a distinct protein, protein domain, or protein fragment useful for purification, identification, and/or complementation.

As used herein, “TRRAP” and other forms thereof (including “Trrap” and “trap”) refers to the “Transformation/Transcription Domain-Associated Protein” protein, transcript (mRNA), and/or gene expressing said protein from human (NCBI GeneID No. 8295) or from any other mammalian species, including all isoforms and allelic variants thereof. TRRAP is also known as DEDDFA, PAF350/400, PAF400, STAF40, TR-AP, and Tra1. TRRAP may have a cDNA nucleotide sequence which is at least 75% identical, at least 80% identical, at least 85% identical, at least 90% identical, at least 95% identical, at least 98% identical, at least 99% identical or more to SEQ ID NO: 3 or to any other mammalian TRRAP cDNA sequence. TRRAP may have an amino sequence which is at least 75% identical, at least 80% identical, at least 85% identical, at least 90% identical, at least 95% identical, at least 98% identical, at least 99% identical or more to SEQ ID NO: 4 or to any other mammalian TRRAP amino acid sequence. TRRAP may be expressed on its own or may be expressed as a fusion with a MYC or a MYC fragment, with an affinity tag, with a detectable label, and/or with a distinct protein, protein domain, or protein fragment useful for purification, identification, and/or complementation.

As used herein, “MAX” and other forms thereof, refers to the “MYC-associated factor X” protein, transcript (mRNA), and/or gene expression said protein from human (NCBI GeneID No. 4149) or from any other mammalian species, including all isoforms and allelic variants thereof. MAX is also known as bHLHd4. MAX may have a cDNA nucleotide sequence which is at least 75% identical, at least 80% identical, at least 85% identical, at least 90% identical, at least 95% identical, at least 98% identical, at least 99% identical or more to SEQ ID NO: 11 or to any other mammalian MAX cDNA sequence. MAX may have an amino acid sequence which is at least 75% identical, at least 80% identical, at least 85% identical, at least 90% identical, at least 95% identical, at least 98% identical, at least 99% identical or more to SEQ ID NO: 12 or to any other mammalian MAX amino acid sequence. MAX may be expressed on its own or may be expressed as a fusion with a MYC or a MYC fragment, with an affinity tag, with a detectable label, and/or with a distinct protein, protein domain, or protein fragment useful for purification, identification, and/or complementation.

As used herein, a “MYC fragment” refers to any soluble MYC protein fragment from any mammalian species comprising a minimal MYC region defined as a MYC MB2 domain and which is capable of forming a binding interaction with TRRAP or a TRRAP fragment from the same and/or different species. A MYC fragment may be expressed on its own or may be expressed as a fusion with a TRRAP or a TRRAP fragment, with an affinity tag, with a detectable label, and/or with a distinct protein, protein domain, or protein fragment useful for purification, identification, and/or complementation.

As used herein, a “MYC 129-145” fragment, domain, or region (i.e., a “MYC MB2” fragment, domain, or region or “a minimal MYC region”) refers to a MYC protein fragment, domain, or region having an amino acid sequence which is at least 75% identical, at least 80% identical, at least 85% identical, at least 90% identical, at least 95% identical, at least 98% identical, or at least 99% identical to SEQ ID NO: 6 or to a corresponding MYC 129-145 amino acid sequence from a non-human mammalian species obtained by aligning a MYC amino acid sequence from one or more non-human mammalian species with SEQ ID NO: 2 and selecting the amino acid residues which align with amino acid residues 129-145 of SEQ ID NO: 2. A MYC 129-145 fragment may be expressed on its own as an isolated domain or may be expressed as a MYC 129-145 region within a larger MYC fragment or domain. A MYC 129-145 fragment may be expressed as a fusion with a TRRAP or a TRRAP fragment, with an affinity tag, with a detectable label, and/or with a distinct protein, protein domain, or protein fragment useful for purification, identification, and/or complementation. A MYC protein or MYC fragment having the MYC MB2 domain or region deleted (i.e., MYC ΔMB2 or MYC Δ129-145) may be expressed on its own or as a fusion with a TRRAP or a TRRAP fragment, with an affinity tag, with a detectable label, and/or with a distinct protein, protein domain, or protein fragment useful for purification, identification, and/or complementation.

As used herein, a “MYC 1-190” fragment, domain, or region refers to a MYC protein fragment, domain, or region having an amino acid sequence which is at least 75% identical, at least 80% identical, at least 85% identical, at least 90% identical, at least 95% identical, at least 98% identical, or at least 99% identical to SEQ ID NO: 7 or to a corresponding MYC 1-190 amino acid sequence from a non-human mammalian species obtained by aligning a MYC amino acid sequence from one or more non-human mammalian species with SEQ ID NO: 2 and selecting the amino acid residues which align with amino acid residues 1-190 of SEQ ID NO: 2. A MYC 1-190 fragment may be expressed on its own as an isolated domain or may be expressed as a MYC 1-190 region within a larger MYC fragment or domain. A MYC 1-190 fragment may be expressed as a fusion with a TRRAP or a TRRAP fragment, with an affinity tag, with a detectable label, and/or with a distinct protein, protein domain, or protein fragment useful for purification, identification, and/or complementation. A MYC protein or MYC fragment having the MYC 1-190 domain or region deleted (i.e., MYC Δ1-190) may be expressed on its own or as a fusion with a TRRAP or a TRRAP fragment, with an affinity tag, with a detectable label, and/or with a distinct protein, protein domain, or protein fragment useful for purification, identification, and/or complementation.

As used herein, a “MYC 120-161” fragment, domain, or region refers to a MYC protein fragment, domain, or region having an amino acid sequence which is at least 75% identical, at least 80% identical, at least 85% identical, at least 90% identical, at least 95% identical, at least 98% identical, or at least 99% identical to SEQ ID NO: 8 or to a corresponding MYC 120-161 amino acid sequence from a non-human mammalian species obtained by aligning a MYC amino acid sequence from one or more non-human mammalian species with SEQ ID NO: 2 and selecting the amino acid residues which align with amino acid residues 120-161 of SEQ ID NO: 2. A MYC 120-161 fragment may be expressed on its own as an isolated domain or may be expressed as a MYC 120-161 region within a larger MYC fragment or domain. A MYC 120-161 fragment may be expressed as a fusion with a TRRAP or a TRRAP fragment, with an affinity tag, with a detectable label, and/or with a distinct protein, protein domain, or protein fragment useful for purification, identification, and/or complementation. A MYC protein or MYC fragment having the MYC 120-161 domain or region deleted (i.e., MYC Δ120-161) may be expressed on its own or as a fusion with a TRRAP or a TRRAP fragment, with an affinity tag, with a detectable label, and/or with a distinct protein, protein domain, or protein fragment useful for purification, identification, and/or complementation.

As used herein, a “TRRAP fragment” refers to any soluble TRRAP protein fragment from any mammalian species comprising a minimal TRRAP region defined as a TRRAP 2033-2088 region and which is capable of forming a binding interaction with MYC or a MYC fragment from the same and/or different species. A TRRAP fragment may be expressed on its own or may be expressed as a fusion with a MYC or a MYC fragment, with an affinity tag, with a detectable label, and/or with a distinct protein, protein domain, or protein fragment useful for purification, identification, and/or complementation.

As used herein, a “minimal TRRAP region” or “TRRAP 2033-2088 region” refers to an amino acid sequence which is at least 75% identical, at least 80% identical, at least 85% identical, at least 90% identical, at least 95% identical, at least 98% identical, or at least 99% identical to SEQ ID NO: 9 or to a corresponding TRRAP 2033-2088 amino acid sequence from a non-human mammalian species obtained by aligning a TRRAP amino acid sequence from one or more non-human mammalian species with SEQ ID NO: 4 and selecting the amino acid residues which align with amino acid residues 2033-2088 of SEQ ID NO: 4. The TRRAP 2033-2088 region may be expressed on its own as an isolated TRRAP 2033-2088 domain or may be expressed as a TRRAP 2033-2088 region within a larger TRRAP fragment. A TRRAP 2033-2088 fragment may be expressed as a fusion with a MYC or a MYC fragment, with an affinity tag, with a detectable label, and/or with a distinct protein, protein domain, or protein fragment useful for purification, identification, and/or complementation. A TRRAP protein or TRRAP fragment having the TRRAP 2033-2088 domain or region deleted (i.e., TRRAP Δ2033-2088) may be expressed on its own or as a fusion with a MYC or a MYC fragment, with an affinity tag, with a detectable label, and/or with a distinct protein, protein domain, or protein fragment useful for purification, identification, and/or complementation.

As used herein, a “TRRAP 2033-2283” fragment, domain, or region refers to a TRRAP protein fragment, domain, or region having an amino acid sequence which is at least 75% identical, at least 80% identical, at least 85% identical, at least 90% identical, at least 95% identical, at least 98% identical, or at least 99% identical to SEQ ID NO: 10 or to a corresponding TRRAP 2033-2283 amino acid sequence from a non-human mammalian species obtained by aligning a TRRAP amino acid sequence from one or more non-human mammalian species with SEQ ID NO: 4 and selecting the amino acid residues which align with amino acid residues 2033-2283 of SEQ ID NO: 4. A TRRAP 2033-2283 fragment may be expressed on its own as an isolated domain or may be expressed as a TRRAP 2033-2283 region within a larger TRRAP fragment or domain. A TRRAP 2033-2283 fragment may be expressed as a fusion with a MYC or a MYC fragment, with an affinity tag, with a detectable label, and/or with a distinct protein, protein domain, or protein fragment useful for purification, identification, and/or complementation. A TRRAP protein or TRRAP fragment having the TRRAP 2033-2283 domain or region deleted (i.e., TRRAP Δ2033-2283) may be expressed on its own or as a fusion with a MYC or a MYC fragment, with an affinity tag, with a detectable label, and/or with a distinct protein, protein domain, or protein fragment useful for purification, identification, and/or complementation.

### II. Methods for Identifying an Inhibitor of an Interaction Between MYC and TRRAP

### A. Identification and Characterization of a MYC:TRRAP Binding Interaction

A method for identifying an inhibitor of an interaction between the oncogenic transcription factor MYC and its cofactor TRRAP is provided. In general, the method involves forming a MYC:TRRAP complex having a MYC:TRRAP binding interaction, directly and/or indirectly detecting the MYC:TRRAP complex and/or the MYC:TRRAP binding interaction to determine a baseline measurement for the MYC:TRRAP complex and/or the MYC:TRRAP binding interaction, introducing a chemical compound prior to or after forming a MYC:TRRAP complex having a MYC:TRRAP binding interaction, and determining an absence or a reduction of the MYC:TRRAP complex and/or the MYC:TRRAP binding interaction after the chemical compound has been introduced compared to the baseline measurement, wherein the absence or the reduction of the MYC:TRRAP complex and/or the MYC:TRRAP binding interaction indicates that the chemical compound is an inhibitor of the binding interaction between MYC and TRRAP.

### B. Cell-Based Methods

The general method described above may include a cell-based method for forming and identifying a MYC:TRRAP binding interaction, and for screening chemical compounds for an ability to inhibit a binding interaction between MYC and TRRAP. The cell-based methods may include a protein-fragment complementation assay (PCA). PCA is a method for the identification and quantification of protein-protein interactions. In the PCA, the proteins of interest (“bait” and “prey”) are each covalently linked to fragments of a third protein which acts as a reporter. Interaction between the bait and the prey proteins brings the fragments of the reporter protein in close proximity to allow them to form a functional reporter protein whose activity can be measured. This principle can be applied to many different reporter proteins and is the basis for PCA assays such as the yeast two-hybrid system, an archetypical PCA assay.

The protein-fragment complementation assay may also be a luminescence complementation assay, namely a split luciferase system called NanoLuc® Binary Technology (NanoBiT®), developed by Promega Corporation. This assay was established using a novel 19.1 kDa, monomeric, highly soluble and stable, ATP-independent luciferase enzyme called NanoLuc® as the reporter protein (65). The NanoLuc® enzyme was split into two parts: Large BiT (LgB; 18 kDa) and Small BiT (SmB; 11 amino acid peptide). These are used as tags on the two proteins of interest; upon protein dimerization, the tags complement and form a highly active luciferase enzyme.

Using the minimal domains that form the MYC and TRRAP complex, each grafted to the LgB and SmB tags, an in-cell luminescence complementation system was developed that can be used to measure direct binding interactions of MYC and TRRAP mutants or the inhibition of binding by small-molecule chemical compounds.

The cell may be selected from a human cell, a mammalian cell, an insect cell, a yeast cell, and a bacterial cell. The cell-based methods may also include cells within a non-human animal selected from C. elegans, D. melanogaster, a zebrafish, a rodent, and a non-human primate.

The cell-based methods may also include cell-based and in vitro steps, such as co-purification of endogenous MYC and TRRAP from cell lysate. The cell-based methods may include cellular co-expression and co-purification of exogenous MYC and TRRAP, MYC and TRRAP fragments, or a MYC-TRRAP fusion from cell lysate. The cell-based methods may include cellular co-expression and co-immunoprecipitation of tagged MYC and TRRAP from cell lysate.

### C. In Vitro Methods

The general method described above may include an in vitro method for forming and identifying a MYC:TRRAP binding interaction, and for screening chemical compounds for an ability to inhibit a binding interaction between MYC and TRRAP. The in vitro approaches may include formation and detection of a MYC:TRRAP complex in any in vitro environment and may comprise any protein-protein interaction assay known in the art. For example, the in vitro methods may include a pulldown assay, an NMR assay, an intrinsic fluorescence assay, a biomolecular fluorescence complementation (BiFC) assay, size exclusion chromatography, a bioluminescence resonance energy transfer (BRET) assay, a fluorescence resonance energy transfer (FRET) assay, a fluorescence polarization (FP) and/or fluorescence anisotropy (FA) assay, surface plasmon resonance (SPR), native polyacrylamide gel electrophoresis (PAGE), a protein microarray, a microfluidic assay, and electron microscopy.

The in vitro methods may further comprise a MYC-TRRAP fusion having a linker with a unique protease cleavage site, such as a 3C protease cleavage site or a TEV protease cleavage site. The in vitro methods may also include a protein-stabilizing additive, such as ethylene glycol (EG), 2,2,2-trifluoroethanol (TFE), and deuterated TFE (TFE-d2), or any combination of these. The identity and concentration of protein-stabilizing additive may be determined using circular dichroism. For example, protein-stabilizing additive may have a concentration ranging from about 5% (v/v) to about 50% (v/v), or from about 20% (v/v) to about 30% (v/v).

### D. In Silico Methods

It is also contemplated that the methods may involve in silico computational analysis of the MYC:TRRAP complex and in silico screening of chemical compounds for an ability to disrupt the MYC:TRRAP complex.

### E. Chemical Compounds and Derivatives Thereof

Also provided are compounds for use as inhibitors of an interaction between MYC and TRRAP, and methods for developing a cancer therapeutic from such compounds, including methods for derivatizing such inhibitors and for testing the inhibitors and derivatized inhibitors for an ability to treat cancer in a subject. The methods, compounds, and compositions provided herein can provide various advantages, such as a means to target the oncogenic transcription factor MYC in cancer.

The chemical compound may be selected from any small-molecule organic chemical compound. The chemical compound may be selected from a chemical compound library, such as from the NCI/DTP Open Chemical Repository. Examples are described below.

Approved Oncology Drugs Set VIII: A set of FDA-approved anticancer drugs consisting of 133 agents.

Diversity Set VI: The Diversity Set VI consists of 1584 compounds derived from 140,000 compounds using the programs Chem-X (Oxford Molecular Group) and Catalyst (Accelrys, Inc.). These programs use defined pharmacophoric centers and defined distance intervals to create a finite set of three dimensional, 3-point pharmacophores resulting in over 1,000,000 possible pharmacophores.

Mechanistic Set IV: The Mechanistic Set IV consists of 811 compounds derived from 37,836 compounds that have been tested in the NCI human tumor 60 cell line screen. This mechanistic diversity set was chosen to represent a broad range of growth inhibition patterns.

Natural Products Set IV: The Natural Products Set IV consists of 419 compounds selected by origin, purity, structural diversity, and availability of compound.

Chemical compounds for use as inhibitors of an interaction between MYC and TRRAP may include any of the compounds listed in Table 1 and derivatives thereof,

TABLE 1
Exemplary Chemical Compounds Which Inhibit MYC/TRRAP Interaction

| Pri- | | | Molec- | | | |
|---|---|---|---|---|---|---|
| ority | % Inhibition | | ular | | | |
| Rank | 10 uM | NSC | Weight | IUPAC | InChI (image) | InChI |
| 1 | 14.38457729 | 106208 | 248 | 3-[(2- chlorophenyl) diazenyl] pyridine-2,6- diamine | 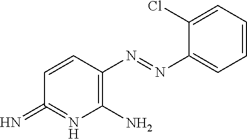 | InChI = 1S/C11H10ClN5/ c12- 7-3-1-2-4-8(7)16-17-9-5-6- 10(13)15-11(9)14/h1- 6H,(H4,13,14,15) |
| 2 | 11.16107149 | 2186 | 431 | 3,3-bis(4-hydroxy- 2-methyl-5-propan- 2-ylphenyl)-2- benzofuran-1-one | 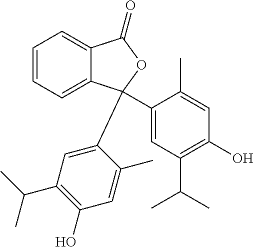 [SMILES: Cc1cc(O)c(C(C)C)cc1C1(c2cc(C(C)C)c(O)cc2C)OC(=O)c2ccccc21] | InChI = 1S/C28H30O4/c1- 15(2)20-13-23(17(5)11- 25(20)29)28(22-10-8-7-9- 19(22)27(31)32-28)24-14- 21(16(3)4)26(30)12- 18(24)6/h7-16,29-30H,1- 6H3 |
| 3 | 22.40657061 | 13974 | 247 | 4- phenyldiazenylnaph- thalen-1-amine | 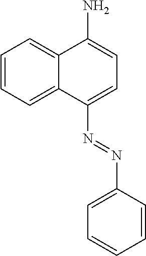 [SMILES: Nc1ccc(/N=N/c2ccccc2)c2ccccc12] | InChI = 1S/C16H13N3/c17- 15-10-11-16(14-9-5-4-8- 13(14)15)19-18-12-6-2-1-3- 7-12/h1-11H,17H2 |
| 4 | 30.35289092 | 108235 | 229 | 4-[(2,6- diaminopyridin-3- yl)diazenyl]phenol | 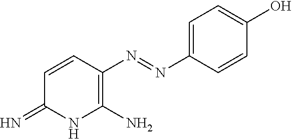 [SMILES: N=c1ccc(/N=N/c2ccc(O)cc2)c(N)[nH]1] | InChI = 1S/C11H11N5O/c12- 10-6-5-9(11(13)14-10)16- 15-7-1-3-8(17)4-2-7/h1- 6,17H,(H4,12,13,14) |
| 5 | 37.23823552 | 374898 | 411 | N-(2,4- dimethylphenyl)-3- hydroxy-4-[(2- hydroxyphenyl) diazenyl] naphthalene- 2-carboxamide | 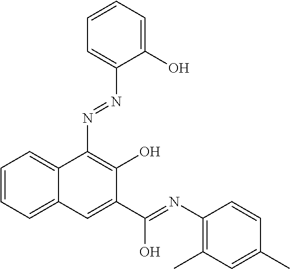 [SMILES: Cc1ccc(/N=C(\O)c2cc3ccccc3c(/N=N/c3ccccc3O)c2O)c(C)c1] | InChI = 1S/C25H21N3O3/ c1- 15-11-12-20(16(2)13-15)26- 25(31)19-14-17-7-3-4-8- 18(17)23(24(19)30)28-27- 21-9-5-6-10-22(21)29/h3- 14,29-30H,1-2H3,(H,26,31) |
| 6 | 33.65552204 | 71300 | 402 | 2-(2,5- dimethoxyphenyl)- 5,6,7,8- tetramethoxy- chromen-4-one | 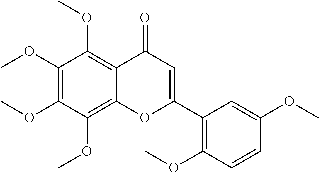 | InChI = 1S/C21H22O8/c1-23- 11-7-8-14(24-2)12(9-11)15- 10-13(22)16-17(25-3)19(26- 4)21(28-6)20(27- 5)18(16)29-15/h7-10H,1- 6H3 |
| 7 | 16.01835024 | 679524 | 533 | 2-O-benzyl 8-O- methyl 3-(4- methylphenyl) sulfonyl- 4,5-dioxo-6H- pyrrolo[3,2- e]indole-2,8- dicarboxylate | 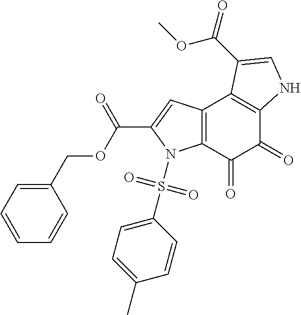 [SMILES: COC(=O)c1c[nH]c2c1-c1cc(C(=O)OCc3ccccc3)n(S(=O)(=O)c3ccc(C)cc3)c1C(=O)C2=O] | InChI = 1S/C27H20N2O8S/ c1-15-8-10-17(11-9- 15)38(34,35)29- 20(27(33)37-14-16-6-4-3-5- 7-16)12-18-21-19(26(32)36- 2)13-28- 22(21)24(30)25(31)23(18)29/ h3-13,28H,14H2,1-2H3 |
| 8 | 33.32495792 | 679527 | 479 | methyl 1-(4- methylphenyl) sulfonyl-4-(4- phenylmethoxycar- bonyl-1H-pyrrol-3- yl)pyrrole-2- carboxylate | 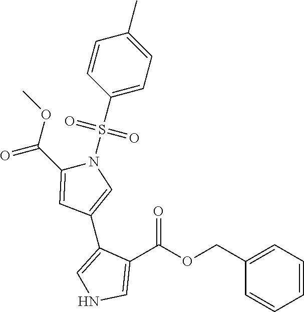 [SMILES: COC(=O)c1cc(-c2c[nH]cc2C(=O)OCc2ccccc2)cn1S(=O)(=O)c1ccc(C)cc1] | InChI = 1S/C25H22N2O6S/ c1-17-8-10-20(11-9- 17)34(30,31)27-15-19(12- 23(27)25(29)32-2)21-13-26- 14-22(21)24(28)33-16-18-6- 4-3-5-7-18/h3- 15,26H,16H2,1-2H3 |
| 9 | 48.58727868 | 37187 | 312 | N-(5-chloro-2- methylphenyl)-3- hydroxy- naphthalene- 2-carboxamide | 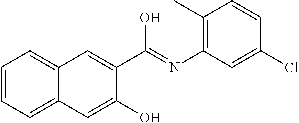 [SMILES: Cc1ccc(Cl)cc1/N=C(\O)c1cc2ccccc2cc1O] | InChI = 1S/C18H14ClNO2/ c1-11-6-7-14(19)10-16(11) 20-18(22)15-8-12-4-2-3-5- 13(12)9-17(15)21/h2- 10,21H,1H3,(H,20,22) |
| 10 | 58.97645483 | 657456 | 399 | 3-[[4-(4- bromophenyl)-1,3- thiazol-2- yl]diazenyl]-1H- indol-2-ol | 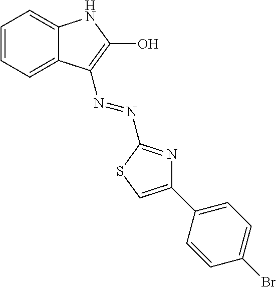 [SMILES: Oc1[nH]c2ccccc2c1/N=N/c1nc(-c2ccc(Br)cc2)cs1] | InChI = 1S/C17H11BrN4OS/ c18-11-7-5-10(6-8-11)14- 9-24- 17(20-14)22-21-15-12-3-1- 2-4-13(12)19-16(15)23/h1- 9,19,23H |
| 11 | 46.89103186 | 111041 | 189.17 | 7-amino-5-imino- 1H-quinoline-2,8- dione | 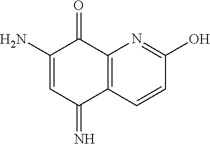 [SMILES: N=C1C=C(N)C(=O)c2nc(O)ccc21] | InChI = 1S/C9H7N3O2/ c10-5- 3-6(11)9(14)8-4(5)1-2- 7(13)12-8/h1- 3,10H,11H2,(H,12,13) |
| 12 | 58.86073027 | 680516 | 290 | N,N,3-trimethyl-4- (quinolin-6- yldiazenyl)aniline | 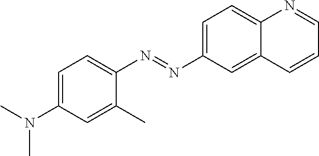 [SMILES: Cc1cc(N(C)C)ccc1/N=N/c1ccc2ncccc2c1] | InChI = 1S/C18H18N4/ c1-13- 11-16(22(2)3)7-9-17(13)21- 20-15-6-8-18-14(12-15)5-4- 10-19-18/h4-12H,1-3H3 |
| 13 | 68.66349117 | 659501 | 258 | 3-methyl-2- methyliminobenzo [f][1,3] benzothiazole- 4,9-dione | 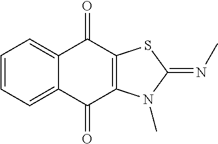 [SMILES: C/N=c1\sc2c(n1C)C(=O)c1ccccc1C2=O] | InChI = 1S/C13H10N2O2S/ c1- 14-13-15(2)9-10(16)7-5-3-4- 6-8(7)11(17)12(9)18-13/h3- 6H,1-2H3 |
| 14 | 40.77706328 | 45383 | 506.47 | 5-amino-6-(7- amino-6-methoxy- 5,8-dioxoquinolin- 2-yl)-4-(2-hydroxy- 3,4- dimethoxyphenyl)- 3-methylpyridine- 2-carboxylic acid | 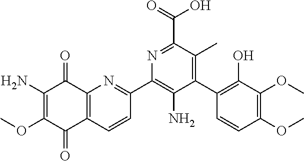 | InChI = 1S/C25H22N4O8/ c1- 9-14(10-6-8-13(35-2)23(36- 3)20(10)30)15(26)19(29- 17(9)25(33)34)12-7-5-11- 18(28- 12)22(32)16(27)24(37- 4)21(11)31/h5-8,30H,26- 27H2,1-4H3,(H,33,34) |
| 15 | 57.01810981 | 359463 | 644 | 1,5-bis[[3- (diethyl- aminomethyl)- 4-hydroxyphenyl] diazenyl] naphthalene-2,6- diol; hydrochloride | 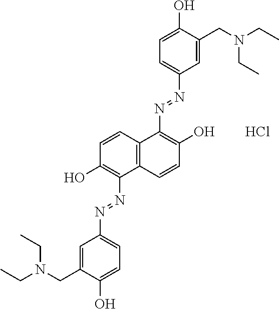 [SMILES: CCN(CC)Cc1cc(/N=N/c2c(O)ccc3c(/N=N/c4ccc(O)c(CN(CC)CC)c4)c(O)ccc23)ccc1O ; Cl] | InChI = 1S/ C32H38N6O4•ClH/ c1-5-37(6-2)19-21-17-23(9- 13-27(21)39)33-35-31-25- 11-16-30(42)32(26(25)12- 15-29(31)41)36-34-24-10- 14-28(40)22(18-24)20-38(7- 3)8-4;/h9-18,39-42H,5-8,19- 20H2,1-4H3;1H |
| 16 | 62.39884673 | 40749 | 290 | (5-methyl-2- phenyldiazenyl-1H- imidazol-4-yl)- phenyldiazene | 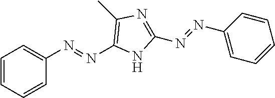 [SMILES: Cc1nc(/N=N/c2ccccc2)[nH]c1/N=N/c1ccccc1] | InChI = 1S/C16H14N6/c1- 12- 15(21-19-13-8-4-2-5-9- 13)18-16(17-12)22-20-14- 10-6-3-7-11-14/h2- 11H,1H3,(H,17,18) |
| 17 | 46.81378485 | 248605 | 715 | 1-[(2S)-6-[(6S)-5- (1-acetyloxyethyl)- 1,6- dihydroxy-6- methyl-8,9,10- trioxo-5,7- dihydroanthracen- 2-yl]-2,5- dihydroxy- 2-methyl-4,9,10- trioxo-1,3- dihydroanthracen- 1-yl]ethyl acetate | 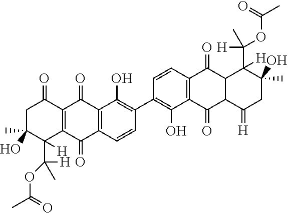 | InChI = 1S/C38H34O14/c1- 13(51-15(3)39)29-27- 25(21(41)11- 37(29,5)49)35(47)23- 19(33(27)45)9-7- 17(31(23)43)18-8-10-20- 24(32(18)44)36(48)26- 22(42)12- 38(6,50)30(28(26)34(20)46) 14(2)52-16(4)40/h7-10,13- 14,29-30,43-44,49-50H,11- 12H2,1- 6H3/t13?,14?,29?,30?,37-, 38-/m0/s1 |
| 18 | 57.20547726 | 108753 | 209 | 2-(6-methyl-2,3- dihydro-1H- quinolin-4- ylidene)propanedi- nitrile | 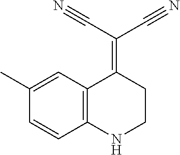 [SMILES: Cc1ccc2c(c1)C(=C(C#N)C#N)CCN2] | InChI = 1S/C13H11N3/ c1-9-2- 3-13-12(6-9)11(4-5-16- 13)10(7-14)8-15/h2- 3,6,16H,4-5H2,1H3 |
| 19 | 92.00826949 | 369317 | 180 | 1-cyano-N-(4- fluorophenyl) methanethioamide | 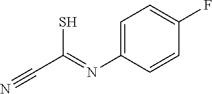 [SMILES: N#C/C(S)=N/c1ccc(F)cc1] | InChI = 1S/C8H5FN2S/ c9-6-1- 3-7(4-2-6)11-8(12)5-10/h1- 4H,(H,11,12) |
| 20 | 41.40164371 | 18268 | 3808 | 2-amino-1-N- [(3S,6S,7R,10S, 16S)- 3-[(2S)-butan-2- yl]-7,11,14- trimethyl- 2,5,9,12,15- pentaoxo-10- propan-2-yl-8-oxa- 1,4,11,14- tetrazabicyclo [14.3.0] nonadecan-6-yl]- 4,6-dimethyl-3- | 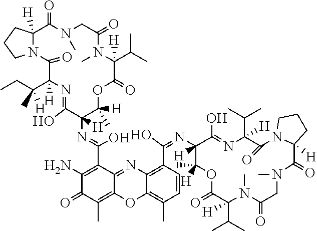 [SMILES: CC[C@H](C)[C@@H]1/N=C(\O)[C@@H](/N=C(\O)c2c3nc4c(/C(O)=N\[C@@H]5/C(O)=N/[C@@H](C(C)C)C(=O)N6CCC[C@H]6C(=O)N(C)CC(=O)N(C)[C@@H](C(C)C)C(=O)O[C@@H]5C)ccc(C)c4oc-3c(C)c(=O)c2N)[C@@H](C)OC(=O)[C@H](C(C)C)N(C)C(=O)CN(C)C(=O)[C@@H]2CCCN2C1=O] | InChI = 1S/ C63H88N12O16/ c1-17-31(8)44-61(86)75-25- 19-21- 38(75)59(84)71(14)27- 40(77)73(16)50(30(6)7)63 (88)90-35(12)46(57(82) 67-44)69-55(80)41- 42(64)51(78)33(10)53- 48(41)65-47-36(23-22- 32(9)52(47)91-53)54(79) 68-45-34(11)89- 62(87)49(29(4)5)72(15)39 (76)26-70(13)58(83)37- |
| | | | | oxo-9-N- | | 20-18-24- |
| | | | | [(3S,6S,7R, | | 74(37)60(85)43(28(2)3)66- |
| | | | | 10S,16S)- | | 56(45)81/h22-23,28-31,34- |
| | | | | 7,11,14-trimethyl- | | 35,37-38,43-46,49-50H,17- |
| | | | | 2,5,9,12,15- | | 21,24-27,64H2,1- |
| | | | | pentaoxo-3,10- | | 16H3,(H,66,81)(H,67,82)(H, |
| | | | | di(propan-2-yl)-8- | | 68,79)(H,69,80)/t31-, |
| | | | | oxa-1,4,11,14- | | 34+,35+,37-,38-,43-,44-,45-, |
| | | | | tetrazabicyclo | | 46-,49-,50-/m0/s1 |
| | | | | [14.3.0] | | |
| | | | | nonadecan-6- | | |
| | | | | yl]phenoxazine-1,9- | | |
| | | | | dicarboxamide | | |
| 21 | 39.87934306 | 679525 | 533 | 8-O-benzyl 2-O- methyl 3-(4- methylphenyl) sulfonyl- 4,5-dioxo-6H- pyrrolo[3,2- e]indole-2,8- dicarboxylate | 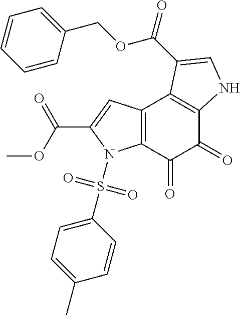 [SMILES: COC(=O)c1cc2c(n1S(=O)(=O)c1ccc(C)cc1)C(=O)C(=O)c1[nH]cc(C(=O)OCc3ccccc3)c1-2] | InChI = 1S/ C27H20N2O8S/ c1-15-8-10-17(11-9- 15)38(34,35)29- 20(27(33)36-2)12-18-21- 19(13-28- 22(21)24(30)25(31)23 (18)29) 26(32)37-14-16-6-4-3-5-7- 16/h3-13,28H,14H2,1-2H3 |
| 22 | 52.47806631 | 93419 | 575 | 4-(4,5-dihydroxy- 3-methoxy-6- methyloxan-2- yl)oxy-2,5,7- trihydroxy-3,9- dimethoxy-2- methyl-3,4- dihydrotetracene- 1,6,11-trione | 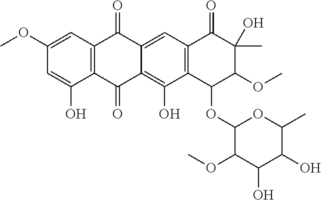 [SMILES: COc1cc(O)c2c(c1)C(=O)c1cc3c(c(O)c1C2=O)C(OC1OC(C)C(O)C(O)C1OC)C(OC)C(C)(O)C3=O] | InChI = 1S/C28H30O13/ c1-9- 18(30)22(34)24(38-4) 27(40-9)41-23-17- 13(25(35)28(2,36)26(23)39- 5)8-12- 16(21(17)33)20(32)15- 11(19(12)31)6-10(37-3)7- 14(15)29/h6-9,18,22-24,26- 27,29-30,33-34,36H,1-5H3 |
| 23 | 50.17280125 | 45384 | 520.5 | methyl 5-amino-6- (7-amino-6- methoxy-5,8- dioxoquinolin-2- yl)-4-(2- hydroxy-3,4- dimethoxyphenyl)- 3-methylpyridine- 2-carboxylate | 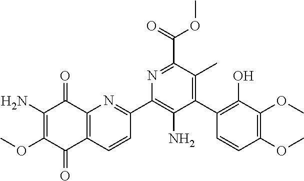 | InChI = 1S/C26H24N4O8/ c1- 10-15(11-7-9-14(35- 2)24(36- 3)21(11)31)16(27)20(30- 18(10)26(34)38-5)13-8-6- 12-19(29- 13)23(33)17(28)25(37- 4)22(12)32/h6-9,31H,27- 28H2,1-5H3 |
| 24 | 41.01460284 | 307981 | 434 | 6-hydroxy-7-(4- hydroxyphenyl)-5- methoxy-2,2- dimethyl-10-(3- methylbut-2- enyl)pyrano[3,2- g]chromen-8-one | 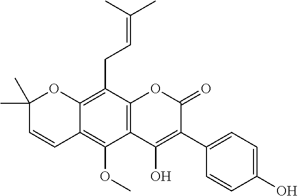 [SMILES: COc1c2c(c(CC=C(C)C)c3oc(=O)c(-c4ccc(O)cc4)c(O)c13)OC(C)(C)C=C2] | lnChI = 1S/C26H26O6/c1- 14(2)6-11-17-22-18(12-13- 26(3,4)32-22)23(30-5)20- 21(28)19(25(29)31- 24(17)20)15-7-9-16(27)10- 8-15/h6-10,12-13,27- 28H,11H2,1-5H3 |
| 25 | 42.51816789 | 242557 | 282 | 2-[(3,5-ditert-butyl- 4- hydroxyphenyl) methylidene] propanedinitrile | 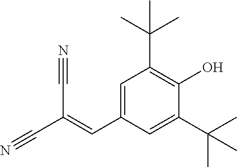 [SMILES: CC(C)(C)c1cc(C=C(C#N)C#N)cc(C(C)(C)C)c1O] | InChI = 1S/C18H22N2O/c1- 17(2,3)14-8-12(7-13(10- 19)11-20)9- 15(16(14)21)18(4,5)6/h7- 9,21H,1-6H3 |
| 26 | 90.89043061 | 369318 | 190 | 1-cyano-N-(2,4- dimethylphenyl) methanethioamide | 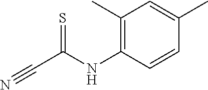 | InChI = 1S/ C10H10N2S/c1-7- 3-4-9(8(2)5-7)12-10(13)6- 11/h3-5H,l-2H3,(H,12,13) |
| 27 | 75.42496379 | 18298 | 572 | 3-[18-(2- carboxyethyl)- 3,8,13,17- tetramethyl-22,23- dihydroporphyrin- 2-yl]propanoic acid | 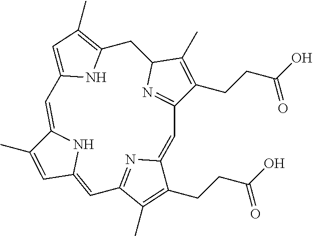 [SMILES: CC1=C(CCC(=O)O)/C2=C/C3=NC(Cc4[nH]c(cc4C)/C=c4\[nH]/c(cc4C)=C\C1=N2)C(C)=C3CCC(=O)O] | InChI = 1S/ C30H30N4O4/c1- 15-9-20-12-25-17(3)21(5-7- 29(35)36)27(33-25)14-28- 22(6-8- 30(37)38)18(4)26(34-28)13- 24-16(2)10-19(32-24)11- 23(15)31-20/h9-14,31- 32H,5-8H2,1- 4H3,(H,35,36)(H,37,38) |
| 28 | 66.32362424 | 631529 | 341 | 2-(4- chloroanilino)benzo [f][1,3] benzothiazole- 4,9-dione | 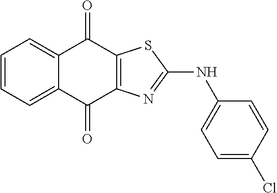 [SMILES: O=C1c2ccccc2C(=O)c2sc(Nc3ccc(Cl)cc3)nc21] | InChI = 1S/ C17H9ClN2O2S/ c18-9-5-7-10(8-6-9)19-17- 20-13-14(21)11-3-1-2-4- 12(11)15(22)16(13)23- 17/h1-8H,(H,19,20) |
| 29 | 60.10082935 | 45384 | 520.5 | methyl 5-amino-6- (7-amino-6- methoxy-5,8- dioxoquinolin-2- yl)-4-(2- hydroxy-3,4- dimethoxyphenyl)- 3-methylpyridine- 2-carboxylate | 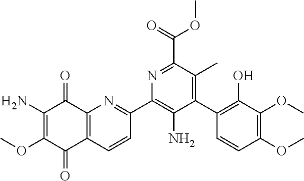 | InChI = 1S/C26H24N4O8/ c1-10-15(11-7-9-14(35- 2)24(36- 3)21(11)31)16(27)20(30- 18(10)26(34)38-5)13-8-6- 12-19(29- 13)23(33)17(28)25(37- 4)22(12)32/h6-9,31H,27- 28H2,1-5H3 |
| 30 | 68.67934757 | 785144 | 351.35 | missing | | |
| 31 | 68.27370242 | 56817 | 519 | 7-(8-formyl-1,6,7- trihydroxy-3- methyl-5-propan-2- ylnaphthalen-2-yl)- 2,3,8-trihydroxy-6- methyl-4-propan-2- ylnaphthalene-1- carbaldehyde | 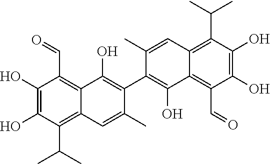 [SMILES: Cc1cc2c(C(C)C)c(O)c(O)c(C=O)c2c(O)c1-c1c(C)cc2c(C(C)C)c(O)c(O)c(C=O)c2c1O] | InChI = 1S/C30H30O8/c1- 11(2)19-15-7- 13(5)21(27(35)23(15)17(9- 31)25(33)29(19)37)22- 14(6)8-16- 20(12(3)4)30(38)26(34)18 (10-32)24(16)28(22)36/h7- 12,33-38H,1-6H3 |
| 32 | 80.85334594 | 177407 | 333.09 | 5,6-dichloro-2-[3- (trifluoromethyl) phenyl]-1H- imidazo[4,5- b]pyrazine | 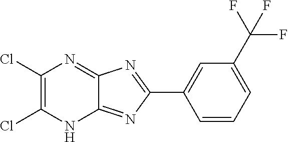 [SMILES: FC(F)(F)c1cccc(-c2nc3nc(Cl)c(Cl)[nH]c-3n2)c1] | InChI = 1S/C12H5Cl2F3N4/ c13-7-8(14)19-11-10(18-7) 20-9(21-11)5-2-1-3-6(4- 5)12(15,16)17/h1- 4H,(H,18,19,20,21) |
| 33 | 29.74327127 | 179834 | 487 | (1S,2R,7S,9R,11R, 12R,15S,16S)-15- [(1S)-1-[(2S)-4,5- dimethyl-6-oxo- 2,3-dihydropyran- 2-yl]-1- hydroxyethyl]- 12,15-dihydroxy- 2,16-dimethyl-8- oxapentacyclo [9.7.0.02, 7.07,9.012,16] | 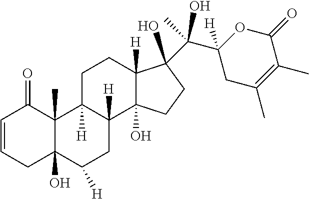 | InChI = 1S/C28H38O7/c1- 15-13-20(34- 22(30)16(15)2)25(5,31)28 (33)12-11-26(32)18-14-21- 27(35-21)9-6-7- 19(29)24(27,4)17(18)8-10- 23(26,28)3/h6-7,17-18,20- 21,31-33H,8-14H2,1- 5H3/t17-,18+,20-,21+,23-, 24-,25-,26+,27+,28-/m0/s1 |
| | | | | octadec-4-en-3-one | | |
| 34 | 78.65192228 | 268242 | 744 | (7S,9S)-9-acetyl-7- [4- (dibenzylamino)- 5-hydroxy-6- methyloxan-2- yl]oxy-6,9,11- trihydroxy-4- methoxy-8,10- dihydro-7H- tetracene-5,12- dione; hydrochloride | 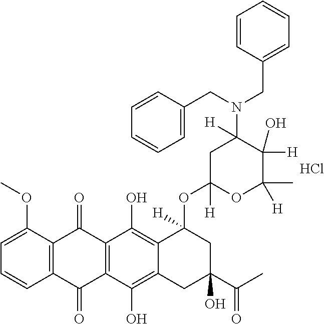 [SMILES: COc1cccc2c1C(=O)c1c(O)c3c(c(O)c1C2=O)C[C@@](O)(C(C)=O)C[C@@H]3OC1CC(N(Cc2ccccc2)Cc2ccccc2)C(O)C(C)O1 ; Cl] | InChI = 1S/ C41H41NO10.ClH/ c1-22-36(44)28(42(20-24- 11-6-4-7-12-24)21-25-13-8- 5-9-14-25)17-31(51-22)52- 30-19-41(49,23(2)43)18-27- 33(30)40(48)35- 34(38(27)46)37(45)26-15- 10-16-29(50-3) 32(26)39(35)47;/h4- 16,22,28,30-31,36,44,46,48- 49H,17-21H2,1- 3H3;1H/t22?,28?,30-, 31?,36?,41-;/m0./s1 |
| 35 | 50.78842551 | 11437 | 302 | (E)-1-(5-chloro-2- hydroxyphenyl)-3- [4-(dimethylamino) phenyl]prop-2-en-1- one | 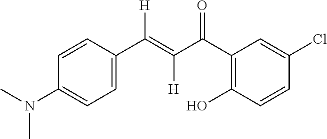 [SMILES: CN(C)c1ccc(/C=C/C(=O)c2cc(Cl)ccc2O)cc1] | InChI = 1S/C17H16ClNO2/ c1-19(2)14-7-3-12(4-8-14) 5-9- 16(20)15-11-13(18)6-10- 17(15)21/h3-11,21H,1- 2H3/b9-5+ |
| 36 | 73.39335453 | 672425 | 397 | (2S)-2-amino-3-(5- oxobenzo[a]pheno- xazin-10- yl)propanoic acid; nitric acid | 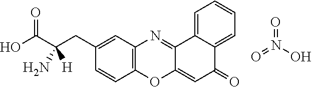 | InChI = 1S/ C19H14N2O4•HNO3/ c20-13(19(23)24)7-10-5- 6-16-14(8-10)21-18-12-4-2- 1-3-11(12)15(22)9- 17(18)25-16;2-1(3)4/h1-6,8- |
| | | | | | | 9,13H,7,20H2,(H,23,24); |
| | | | | | | (H,2,3,4)/t13-;/m0./s1 |
| 37 | 84.83163802 | 85700 | 454.31 | 5-[2-(1,6- dimethylquinolin-1- ium-2- yl)ethenyl]quinolin- 8-ol | 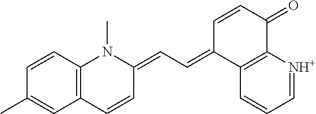 [SMILES: Cc1ccc2c(c1)C=C/C(=C\C=C1/C=CC(=O)c3[nH+]cccc31)N2C] | InChI = 1S/C22H18N2O/c1- 15-5-11-20-17(14-15)7-10- 18(24(20)2)9-6-16-8-12- 21(25)22-19(16)4-3-13-23- 22/h3-14H,1-2H3/p+1 |
| 38 | 76.03001044 | 345647 | 546.53 | 5,6,8-trihydroxy- 2,3-dimethyl-9- 2,3-dimethyl-4- oxo-2,3- dihydrobenzo[g] chromen-9-yl)-2,3- dihydrobenzo[g] chromen-4-one | 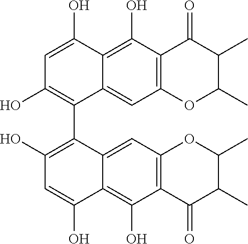 [SMILES: CC1Oc2cc3c(-c4c(O)cc(O)c5c(O)c6c(cc45)OC(C)C(C)C6=O)c(O)cc(O)c3c(O)c2C(=O)C1C] | InChI = 1S/C30H26010/c1- 9-11(3)39-19-5-13- 17(33)23(13)29(37)25(19) 27(9)35)22-14-6-20- 26(28(36)10(2)12(4)40- 20)30(38)24(14)18(34)8- 16(22)32/h5-12,31-34,37- 38H,1-4H3 |
| 39 | 73.41743844 | 785168 | 279.33 | missing | | |
| 40 | 76.05565763 | 26326 | 242.27 | 2,2-dimethyl-3,4- dihydrobenzo[h] chromene-5,6- dione | 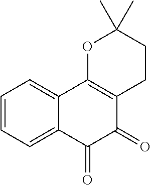 [SMILES: CC1(C)CCC2=C(O1)c1ccccc1C(=O)C2=O] | InChI = 1S/C15H14O3/c1- 15(2)8-7-11-13(17)12(16)9- 5-3-4-6-10(9)14(11)18- 15/h3-6H,7-8H2,1-2H3 |
| 41 | 78.9658571 | 65537 | 356.4 | 3-[(4-amino-3- methoxy- naphthalen-1- yl)diazenyl] benzene- sulfonamide | 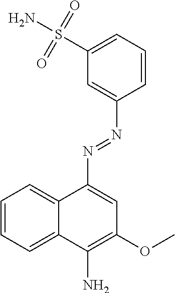 [SMILES: COc1cc(/N=N/c2cccc(S(N)(=O)=O)c2)c2ccccc2c1N] | InChI = 1S/C17H16N4O3S/ c1-24-16-10-15(13-7-2-3- 8-14(13)17(16)18)21-20- 11-5-4-6-12(9- 11)25(19,22)23/h2- 10H,18H2,1H3,(H2,19, 22,23) |
| 42 | 102.4892976 | 72138 | 579 | methyl (1R,15S,17R,18R, 19S,20S)-18- methoxy-17-(3,4,5- trimethoxybenzoyl) oxy- 1,3,11,12,14,15,16, 17,18,19,20,21- dodecahydro- yohimban- 19-carboxylate | 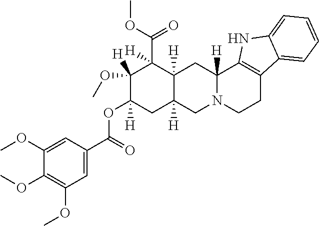 [SMILES: COC(=O)[C@H]1[C@H]2C[C@@H]3c4[nH]c5ccccc5c4CCN3C[C@H]2C[C@@H](OC(=O)c2cc(OC)c(OC)c(OC)c2)[C@@H]1OC] | InChI = 1S/C32H38N2O8/ c1-37-24-12-17(13-25(38- 2)29(24)39-3)31(35)42-26- 14-18-16-34-11-10-20-19-8- 6-7-9-22(19)33- 28(20)23(34)15- 21(18)27(30(26)40- 4)32(36)41-5/h6-9,12- 13,18,21,23,26- 27,30,33H,10-11,14-16H2, 1-5H3/t18-,21+,23-,26-, 27+,30+/m1/s1 |
| 43 | 57.3356708 | 111041 | 189.17 | 7-amino-5-imino- 1H-quinoline-2,8- dione | 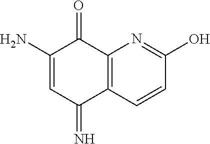 [SMILES: N=C1C=C(N)C(=O)c2nc(O)ccc21] | InChI = 1S/C9H7N3O2/ c10-5-3-6(11)9(14)8-4 (5)1-2-7(13)12-8/h1- 3,10H,11H2,(H,12,13) |
| 44 | 93.68437027 | 11881 | 390 | [3,7- bis(dimethylamino) phenothiazin-10- yl]- phenylmethanone | 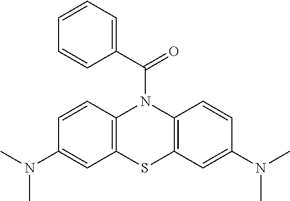 | InChI = 1S/C23H23N3OS/ c1- 24(2)17-10-12-19-21(14- 17)28-22-15-18(25(3)4)11- 13-20(22)26(19)23(27)16-8- 6-5-7-9-16/h5-15H,1-4H3 |
| 45 | 85.81902531 | 250430 | 420.5 | 21-methoxy-17,17- dimethyl-5-(3- methylbut-2-enyl)- 3,12,16- trioxapentacyclo [11.8.0.02,10.04, 9.015,20] henicosa- 1(13),4(9),5,7, 14,18, 20-heptaen-6-ol | 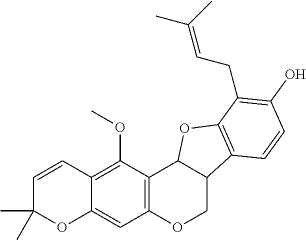 [SMILES: COc1c2c(cc3c1C1Oc4c(ccc(O)c4CC=C(C)C)C1CO3)OC(C)(C)C=C2] | InChI = 1S/C26H28O5/c1- 14(2)6-7-16-19(27)9-8-15- 18-13-29-21-12-20-17(10- 11-26(3,4)31-20)24(28- 5)22(21)25(18)30- 23(15)16/h6,8- 12,18,25,27H,7,13H2,1-5H3 |
| 46 | 96.48476074 | 33575 | 345.44 | (3Z)-3-[[4- (dimethylamino) phenyl] methylidene]- 5-(5,6,7,8- tetrahydro- naphthalen- 2-yl)furan-2-one | 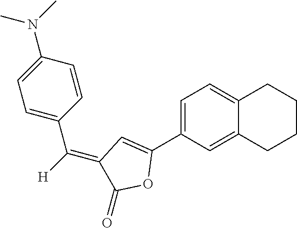 [SMILES: CN(C)c1ccc(/C=C2\C=C(c3ccc4c(c3)CCCC4)OC2=O)cc1] | InCh1= 1S/C23H23NO2/c1- 24(2)21-11-7-16(8-12- 21)13-20-15-22(26- 23(20)25)19-10-9-17-5-3-4- 6-18(17)14-19/h7-15H,3- 6H2,1-2H3/b20-13- |

TABLE 2
Exemplary Chemical Compounds Which Inhibit MYC/TRRAP Interaction

| In- | | | | | | | | |
|---|---|---|---|---|---|---|---|---|
| ternal | | Pri- | | | Molec- | | | |
| Num- | | ority | % Inhibition | | ular | | | |
| bering | CID | Rank | 10 uM | NSC | Weight | IUPAC | InChI | InChI |
| 1024 | 376575 | 1 | 39.63318036 | 657587 | 320 | 3-[(4-phenyl- 1,3-thiazol-2- yl)diazenyl]- 1H-indol-2-ol | 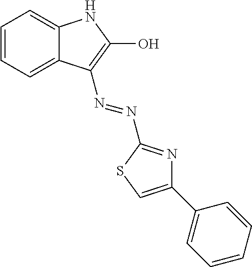 [SMILES: Oc1[nH]c2ccccc2c1/N=N/c1nc(-c2ccccc2)cs1] | InChI = 1S/C17H 12N4OS/c22-16- 15(12-8-4-5-9- 13(12)18-16)20- 21-17-19-14(10- 23-17)11-6-2-1- 3-7-11/h1- 10,18,22H |
| 1023 | 3800162 | 2 | 47.9320828 | 657576 | 365 | 3-[[4-(4- nitrophenyl)- 1,3-thiazol-2- yl]diazenyl]- 1H-indol-2-ol | 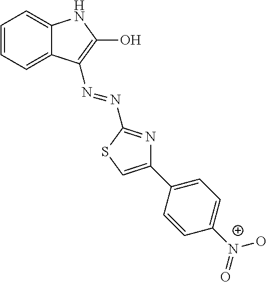 | InChI = 1S/C17H 11N5O3S/c23-16- 15(12-3-1-2-4- 13(12)18-16)20- 21-17-19-14(9- 26-17)10-5-7- 11(8-6- 10)22(24)25/h1- 9,18,23H |
| 1030 | 135499432 | 3 | 48.26266032 | 659825 | 404 | 1-[(E)-2-(1,3- benzothiazol- 2-yl)-2- cyanoethenyl]- 3-[(2- hydroxy-1H- indol-3- yl)imino] thiourea | 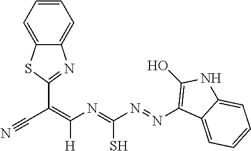 [SMILES: N#C/C(=C/N=C(S)/N=N/c1c(O)[nH]c2ccccc12)c1nc2ccccc2s1] | InChI = 1S/C19H 12N6OS2/c20-9- 11(18-23-14-7- 3-4-8-15(14)28- 18)10-21- 19(27)25-24-16- 12-5-1-2-6- 13(12)22- 17(16)26/h1- 8,10,22,26H,(H,21, 27)/b11- 10+,25-24? |
| 1027 | 376652 | 4 | 50.65631481 | 657698 | 334 | 3-[(5-methyl- 4-phenyl-1,3- thiazol-2- yl)diazenyl]- 1H-indol-2-ol | 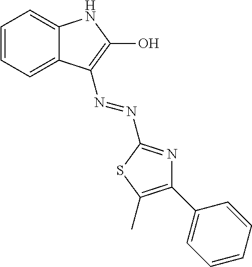 [SMILES: Cc1sc(/N=N/c2c(O)[nH]c3ccccc23)nc1-c1ccccc1] | InChI = 1S/C18H 14N4OS/C1-11- 15(12-7-3-2-4-8- 12)20-18(24- 11)22-21-16-13- 9-5-6-10- 14(13)19- 17(16)23/h2- 10,19,23H,1H4 |
| 1025 | 376577 | 5 | 53.24492022 | 657589 | 389 | 3-[[4-(3,4- dichlorophenyl)- 1,3- thiazol-2- yl]diazenyl]- 1H-indol-2-ol | 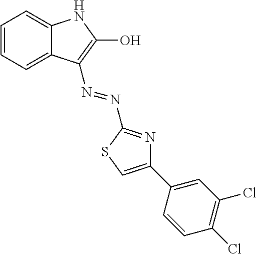 [SMILES: Oc1[nH]c2ccccc2c1/N=N/c1nc(-c2ccc(Cl)c(Cl)c2)cs1] | InChI = 1S/C17H 10Cl2N4OS/c18- 11-6-5-9(7- 12(11)19)14-8- 25-17(21-14)23- 22-15-10-3-1-2- 4-13(10)20- 16(15)24/h1- 8,20,24H |
| 109 | 3776068 | 6 | 54.47523118 | 116536 | 282 | 3-[(4- nitrophenyl) diazenyl]-1H- indol-2-ol | 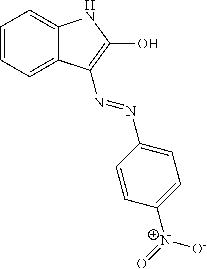 [SMILES: O=[N+]([O-])c1ccc(/N=N/c2c(O)[nH]c3ccccc23)cc1] | InChI =1S/C14H 10N4O3/C19-14- 13(11-3-1-2-4- 12(11)15-14)17- 16-9-5-7-10(8-6- 9)18(20)21/h1- 8,15,19H |
| 1022 | 4412709 | 7 | 54.58339018 | 657568 | 365 | 3-[[4-(3- nitrophenyl)- 1,3-thiazol-2- yl]diazenyl]- 1H-indol-2-ol | 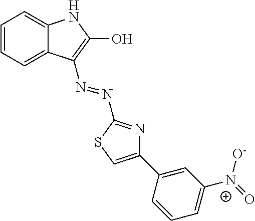 [SMILES: O=[N+]([O-])c1cccc(-c2csc(/N=N/c3c(O)[nH]c4ccccc34)n2)c1] | InChI = 1S/C17H 11N5O3S/c23-16- 15(12-6-1-2-7- 13(12)18-16)20- 21-17-19-14(9- 26-17)10-4-3-5- 11(8- 10)22(24)25/h1- 9,18,23H |
| 1026 | 376595 | 8 | 59.5050642 | 657607 | 330 | ethyl 2-[2- [(2-hydroxy- 1H-indol-3- yl)diazenyl]- 1,3-thiazol-4- yl] acetate | 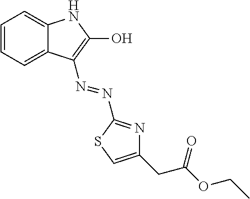 [SMILES: CCOC(=O)Cc1csc(/N=N/c2c(O)[nH]c3ccccc23)n1] | InChI = 1S/C15H 14N4O3S/c1-2- 22-12(20)7-9-8- 23-15(16-9)19- 18-13-10-5-3-4- 6-11(10)17- 14(13)21/h3- 6,8,17,21H,2,7H 2,1H4 |
| 1039 | 135585452 | 9 | 59.83997794 | 728946 | 235 | 5H- indolo[2,3- c][2,1,5]ben- zoxadiazepine | 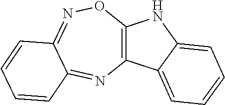 [SMILES: c1ccc2c(c1)=NOc1[nH]c3ccccc3c1N=2] | InChI = 1S/C14H9 N3O/c1-2-6-10- 9(5-1)13-14(16- 10)18-17-12-8- 4-3-7-11(12)15- 13/h1-8,16H |
| 1011 | 272589 | 10 | 64.74626212 | 117192 | 272 | 3-[(2- chlorophenyl) diazenyl]- 1H-indol-2-ol | 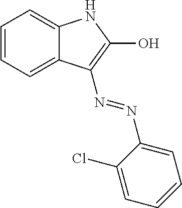 [SMILES: Oc1[nH]c2ccccc2c1/N=N/c1ccccc1Cl] | InChI = 1S/C14H 10ClN3O/C15-10- 6-2-4-8- 12(10)17-18-13- 9-5-1-3-7- 11(9)16- 14(13)19/h1- 8,16,19H |
| 105 | 837253 | 11 | 65.25125763 | 73303 | 296 | 1-[(2- hydroxy-1H- indol-3- yl)imino]-3- phenylthiourea | 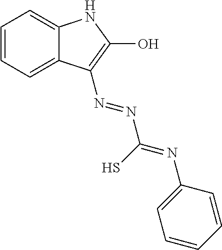 [SMILES: Oc1[nH]c2ccccc2c1/N=N/C(S)=N/c1ccccc1] | InChI = 1S/C15H 12N4OS/c20-14- 13(11-8-4-5-9- 12(11)17-14)18- 19-15(21)16-10- 6-2-1-3-7-10/h1- 9,17,20H,(H,16, 21) |
| 1012 | 272989 | 12 | 66.52969087 | 117808 | 237 | 3- phenyldiazenyl- 1H-indol-2- ol | 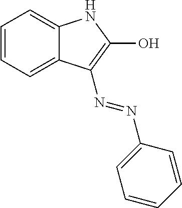 [SMILES: Oc1[nH]c2ccccc2c1/N=N/c1ccccc1] | InChI = 1S/C14H 11N3O/c18-14- 13(11-8-4-5-9- 12(11)15-14)17- 16-10-6-2-1-3-7- 10/h1-9,15,18H |
| 1021 | 2783973 | 13 | 71.88293594 | 635422 | 275 | 4-amino-3- [(2-hydroxy- 1H-indol-3- yl)diazenyl]- 1H-1,2,4- triazole-5- thione | 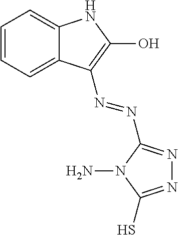 [SMILES: Nn1c(S)nnc1/N=N/c1c(O)[nH]c2ccccc12] | InChI = 1S/C10H9 N7OS/c11-17- 9(15-16- 10(17)19)14-13- 7-5-3-1-2-4- 6(5)12- 8(7)18/h1- 4,12,18H,11H2, (H,16,19) |
| 10 | 376515 | 14 | 73.55903033 | 657456 | 399 | 3-[[4-(4- bromophenyl)- 1,3-thiazol-2- yl]diazenyl]- 1H-indol-2-ol | 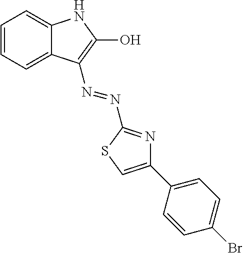 [SMILES: Oc1[nH]c2ccccc2c1/N=N/c1nc(-c2ccc(Br)cc2)cs1] | InChI = 1S/C17H 11BrN4OS/c18- 11-7-5-10(6-8- 11)14-9-24- 17(20-14)22-21- 15-12-3-1-2-4- 13(12)19- 16(15)23/h1- 9,19,23H |
| 1018 | 3000820 | 15 | 77.03571071 | 321199 | 260 | 1-1(2- hydroxy-1H- indol-3- yl)imino]-3- prop-2- enylthiourea | 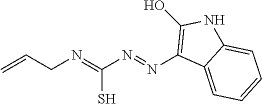 [SMILES: C=CC/N=C(S)/N=N/c1c(O)[nH]c2ccccc12] | InChI = 1S/C12H 12N4OS/c1-2-7- 13-12(18)16-15- 10-8-5-3-4-6- 9(8)14- 11(10)17/h2- 6,14,17H,1,7H2, (H,13,18) |
| 1035 | 772848 | 16 | 82.11898282 | 668501 | 310 | 1-[(2- hydroxy-1H- indol-3- yl)imino]-3- (3- methylphenyl) thiourea | 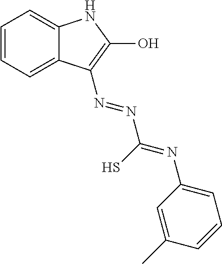 [SMILES: Cc1cccc(/N=C(S)/N=N/c2c(O)[nH]c3ccccc23)c1] | InChI = 1S/C16H 14N4OS/c1-10-5- 4-6-11(9-10)17- 16(22)20-19-14- 12-7-2-3-8- 13(12)18- 15(14)21/h2- 9,18,21H,1H3,(H, 17,22) |
| 1019 | 332489 | 17 | 82.33939147 | 329344 | 257 | 2,2,2- trifluoro-N- [(2-hydroxy- 1H-indol-3- yl)imino] acetamide | 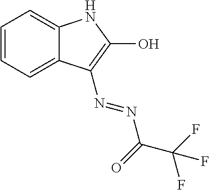 [SMILES: O=C(/N=N/c1c(O)[nH]c2ccccc12)C(F)(F)F] | InChI = 1S/C10H6 F3N3O2/c11- 10(12,13)9(18) 16-15-7-5-3-1-2- 4-6(5)14- 8(7)17/h1- 4,14,17H |
| 1015 | 4300816 | 18 | 82.44008817 | 120144 | 288 | [2-hydroxy-4- (trifluoro- methyl)-1H- indol-3- yl]iminothiourea | 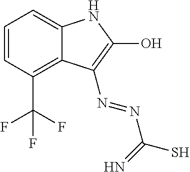 [SMILES: N=C(S)/N=N/c1c(O)[nH]c2cccc(C(F)(F)F)c12] | InChI = 1S/C10H7 F3N4OS/c11- 10(12,13)4-2-1- 3-5- 6(4)7(8(18)15- 5)16-17- 9(14)19/h1- 3,15,18H,(H2,14, 19) |
| 1013 | 273467 | 19 | 83.25403843 | 118728 | 280 | 1-[(2- hydroxy-1H- indol-3- yl)imino]-3- phenylurea | 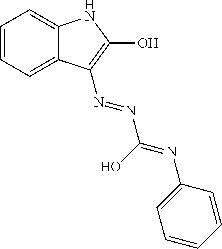 [SMILES: OC(=N\c1ccccc1)/N=N/c1c(O)[nH]c2ccccc12] | InChI = 1S/C15H 12N4O2/c20-14- 13(11-8-4-5-9- 12(11)17-14)18- 19-15(21)16-10- 6-2-1-3-7-10/h1- 9,17,20H,(H,16, 21) |
| 1037 | 381992 | 20 | 85.4883682 | 669319 | 385 | 3-(4,5,6,7- tetrahydro- 1H-1,3- diazepin-2- yldiazenyl)- 1H-indol-2- ol; hydroiodide | 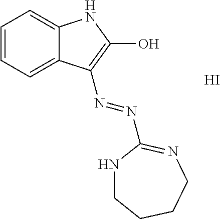 | InChI = 1S/C13H 15N5O•HI/c19- 12-11(9-5-1-2-6- 10(9)16-12)17- 18-13-14-7-3-4- 8-15-13;/h1-2,5- 6,16,19H,3-4,7- 8H2,(H,14,15); 1H |
| 108 | 256526 | 21 | 86.24955041 | 83459 | 248 | 3-1(2- hydroxy-1H- indol-3- yl)imino]-1,1- dimethylthiourea | 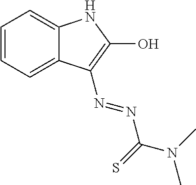 [SMILES: CN(C)C(=S)/N=N/c1c(O)[nH]c2ccccc12] | InChI = 1S/C11H 12N4OS/c1- 15(2)11(17)14- 13-9-7-5-3-4-6- 8(7)12- 10(9)16/h3- 6,12,16H,1-2H4 |
| 1038 | 388108 | 22 | 86.66516352 | 682573 | 279 | N-((2- hydroxy-1H- indol-3- yl)imino]-2- phenylacetamide | 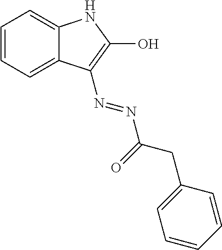 [SMILES: O=C(Cc1ccccc1)/N=N/c1c(O)[nH]c2ccccc12] | InChI = 1S/C16H 13N3O2/c20- 14(10-11-6-2-1- 3-7-11)18-19- 15-12-8-4-5-9- 13(12)17- 16(15)21/h1- 9,17,21H,10H3 |
| 1033 | 381666 | 23 | 86.95546963 | 668494 | 274 | N-((2- hydroxy-1H- indol-3- yl)imino] pyrrolidine-1- carbothioamide | 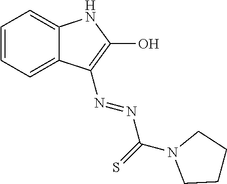 [SMILES: Oc1[nH]c2ccccc2c1/N=N/C(=S)N1CCCC1] | InChI = 1S/C13H 14N4OS/C18-12- 11(9-5-1-2-6- 10(9)14-12)15- 16-13(19)17-7- 3-4-8-17/h1-2,5- 6,14,18H,3-4,7- 8H3 |
| 1036 | 707890 | 24 | 86.97897782 | 668502 | 310 | 1-1(2- hydroxy-1H- indol-3- yl)imino]-3- (4- methylphenyl) thiourea | 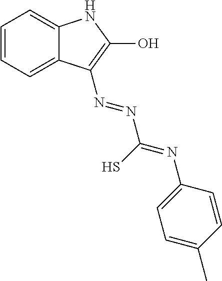 [SMILES: Cc1ccc(/N=C(S)/N=N/c2c(O)[nH]c3ccccc23)cc1] | InChI = 1S/C16H 14N4OS/C1-10-6- 8-11(9-7-10)17- 16(22)20-19-14- 12-4-2-3-5- 13(12)18- 15(14)21/h2- 9,18,21H,1H3,(H, 17,22) |
| 10N | 135499369 | 25 | 88.10443542 | 657457 | 547 | (2E,5Z)-2-[[4- (4- bromophenyl)- 1,3-thiazol-2-yl] hydrazinylidene]- 5-[(3,4,5- trimethoxy- | 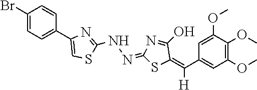 [SMILES: COc1cc(/C=C2/S/C(=N/Nc3nc(-c4ccc(Br)cc4)cs3)N=C2O)cc(OC)c1OC] | InChI = 1S/C22H 19BrN4O4S2/c1- 29-16-8-12(9- 17(30- 2)19(16)31- 3)10-18- 20(28)25-22(33- |
| | | | | | | phenyl) | | 18)27-26-21-24- |
| | | | | | | methylidene]- | | 15(11-32-21)13- |
| | | | | | | 1,3- | | 4-6-14(23)7-5- |
| | | | | | | thiazolidin-4- | | 13/h4-11H,1- |
| | | | | | | one | | 3H3,(H,24,26)(H, |
| | | | | | | | | 25,27,28)/b18- |
| | | | | | | | | 10- |
| 1032 | 3005056 | 26 | 88.62157584 | 668492 | 262 | 1-[(2- hydroxy-1H- indol-3- yl)imino]-3- propylthiourea | 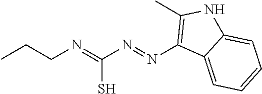 [SMILES: CCC/N=C(S)/N=N/c1c(C)[nH]c2ccccc12] | InChI = 1S/C12H 14N4OS/c1-2-7- 13-12(18)16-15- 10-8-5-3-4-6- 9(8)14- 11(10)17/h3- 6,14,17H,2,7H2, |
| | | | | | | | | 1H3,(H,13,18) |
| 103 | 240794 | 27 | 88.8310143 | 47469 | 374 | 1-N,2-N- bis[(2- hydroxy-1H- indol-3- yl)imino]ethane- diimidamide | 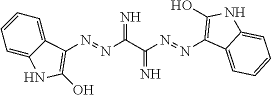 [SMILES: N=C(/N=N/c1c(O)[nH]c2ccccc12)C(=N)/N=N/c1c(O)[nH]c2ccccc12] | InChI = 1S/C18H 14N8O2/c19- 15(25-23-13-9- 5-1-3-7-11(9)21- 17(13)27)16(20) 26-24-14-10-6- 2-4-8-12(10)22- |
| | | | | | | | | 18(14)28/h1- |
| | | | | | | | | 8,19-22,27-28H |
| 1017 | 299560 | 28 | 89.12864369 | 172776 | 237 | N′-(2- hydroxy-1H- indol-3- yl)ethane- diimidoyl dicyanide | 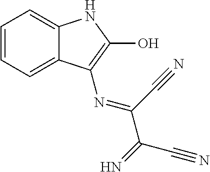 [SMILES: N#CC(=N)/C(C#N)=N/c1c(O)[nH]c2ccccc12] | InChI =1S/C12H7 N5O/c13-5- 8(15)10(6- 14)16-11-7-3-1- 2-4-9(7)17- 12(11)18/h1- 4,15,17-18H |
| 1020 | 341712 | 29 | 91.7232243 | 374728 | 229 | 3-(4,5- dihydro-1H- imidazol-2- yldiazenyl)- 1H-indol-2-ol | 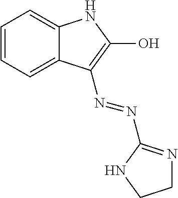 [SMILES: Oc1[nH]c2ccccc2c1/N=N/C1=NCCN1] | InChI = 1S/C11H 11N5O/c17-10- 9(15-16-11-12- 5-6-13-11)7-3-1- 2-4-8(7)14- 10/h1- 4,14,17H,5- 6H2,(H,12,13) |
| 1031 | 135499436 | 30 | 93.29349333 | 659851 | 461 | 1-[(E)-2- benzylsulfinyl- 2- phenylethenyl]- 3-[(2- hydroxy-1H- indol-3- yl)imino] thiourea | 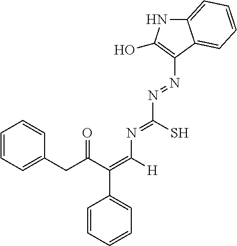 [SMILES: O=C(Cc1ccccc1)\C(=C/N=C(S)/N=N/c1c(O)[nH]c2ccccc12)c1ccccc1] | InChI = 1S/C24H 20N4O2S2/c29- 23-22(19-13-7- 8-14-20(19)26- 23)27-28- 24(31)25-15- 21(18-11-5-2-6- 12-18)32(30)16- 17-9-3-1-4-10- 17/h1- 15,26,29H,16H2, (H,25,31)/b21- 15+,28-27? |
| 1010 | 272588 | 31 | 94.7641608 | 117191 | 251 | 3-[(2- methylphenyl) diazenyl]- 1H-indol-2-ol | 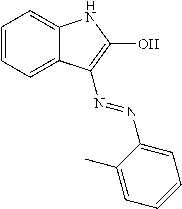 [SMILES: Cc1ccccc1/N=N/c1c(O)[nH]c2ccccc12] | InChI = 1S/C15H 13N3O/c1-10-6- 2-4-8-12(10)17- 18-14-11-7-3-5- 9-13(11)16- 15(14)19/h2- 9,16,19H,1H4 |
| 1028 | 376657 | 32 | 97.4617817 | 657703 | 377 | 2-[2-[(2- hydroxy-1H- indol-3- yl)diazenyl]- 1,3-thiazol-4- y]-N- phenylacetamide | 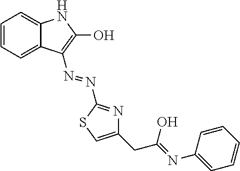 [SMILES: O/C(Cc1csc(/N=N/c2c(O)[nH]c3ccccc23)n1)=N\c1ccccc1] | InChI = 1S/C19H 15N5O2S/c25- 16(20-12-6-2-1- 3-7-12)10-13- 11-27-19(21- 13)24-23-17-14- 8-4-5-9- 15(14)22- 18(17)26/h1- 9,11,22,26H,10H 2,(H,20,25) |
| 1029 | 376659 | 33 | 98.06987707 | 657705 | 391 | 2-2-[(2- hydroxy-1H- indol-3- yl)diazenyl]- 1,3-thiazol-4- yl]-N-(2- methylphenyl) acetamide | 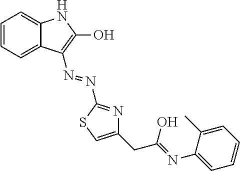 [SMILES: Cc1ccccc1/N=C(\O)Cc1csc(/N=N/c2c(O)[nH]c3ccccc23)n1] | InChI = 1S/C20H 17N5O2S/C1-12- 6-2-4-8- 15(12)22- 17(26)10-13-11- 28-20(21-13)25- 24-18-14-7-3-5- 9-16(14)23- 19(18)27/h2- 9,11,23,27H,10H 2,1H3,(H,22,26) |
| 102 | 135436477 | 34 | 100.4198351 | 1513 | 248 | 1-[(2- hydroxy-1H- indol-3- yl)imino]-3- nitroguanidine | 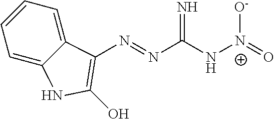 [SMILES: N=C(/N=N/c1c(O)[nH]c2ccccc12)N[N+](=O)[O-]] | InChI = 1S/C9H8N 6O3/c10-9(14- 15(17)18)13-12- 7-5-3-1-2-4- 6(5)11- 8(7)16/h1- 4,11,16H,(H2,10, 14) |
| 101 | 690109 | 35 | 104.452045 | 721 | 220 | (2-hydroxy- 1H-indol-3- yl)iminothiourea | 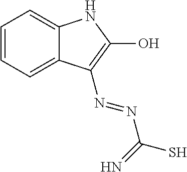 [SMILES: N=C(S)/N=N/c1c(O)[nH]c2ccccc12] | InChI = 1S/C9H8N 4OS/c10- 9(15)13-12-7-5- 3-1-2-4-6(5)11- 8(7)14/h1- 4,11,14H,(H2,10, 15) |
| 1016 | 281526 | 36 | 104.5183687 | 134357 | 260 | 2-[(2- hydroxy-1H- indol-3- yl)diazenyl]- 1,3-thiazol-4- one | 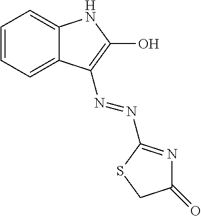 [SMILES: O=C1CSC(/N=N/c2c(O)[nH]c3ccccc23)=N1] | InChI = 1S/C11H8 N4O2S/c16-8-5- 18-11(13-8)15- 14-9-6-3-1-2-4- 7(6)12- 10(9)17/h1- 4,12,17H,5H3 |
| 1014 | 273475 | 37 | 107.2760801 | 118737 | 233 | ethyl N-[(2- hydroxy-1H- indol-3- yl)imino] carbamate | 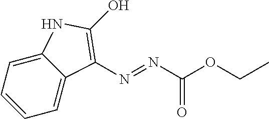 [SMILES: CCOC(=O)/N=N/c1c(O)[nH]c2ccccc12] | InChI = 1S/C11H 11N3O3/c1-2-17- 11(16)14-13-9- 7-5-3-4-6- 8(7)12- 10(9)15/h3- 6,12,15H,2H2, 1H4 |
| 1034 | 2169056 | 38 | 111.8855291 | 668500 | 310 | 1-[(2- hydroxy-1H- indol-3- yl)imino]-3- (2-methylphenyl) thiourea | 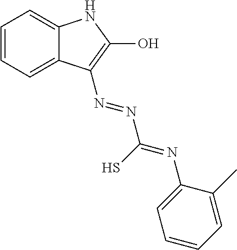 [SMILES: Cc1ccccc1/N=C(S)/N=N/c1c(O)[nH]c2ccccc12] | InChI = 1S/C16H 14N4OS/c1-10-6- 2-4-8-12(10)18- 16(22)20-19-14- 11-7-3-5-9- 13(11)17- 15(14)21/h2- 9,17,21H,1H3,(H, 18,22) |
| 107 | 252921 | 39 | 112.5578699 | 75233 | 204 | (2-hydroxy- 1H-indol-3- yl)iminourea | 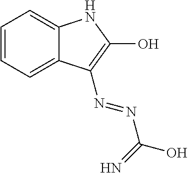 [SMILES: N=C(O)/N=N/c1c(O)[nH]c2ccccc12] | InChI = 1S/C9H8N 4O2/c10- 9(15)13-12-7-5- 3-1-2-4-6(5)11- 8(7)14/h1- 4,11,14H,(H2,10, 15) |
| 104 | 249068 | 40 | 123.875762 | 67024 | 203 | 1-[(2- hydroxy-1H- indol-3- yl)imino] guanidine | 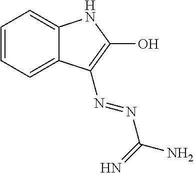 [SMILES: N=C(N)/N=N/c1c(O)[nH]c2ccccc12] | InChI = 1S/C9H9N 5O/c10-9(11)14- 13-7-5-3-1-2-4- 6(5)12- 8(7)15/h1- 4,12,15H,(H3,10, 11) |
| 106 | 252894 | 41 | 127.2795255 | 75201 | 220 | (1,2- dihydroxyindol- 3-yl)iminourea | 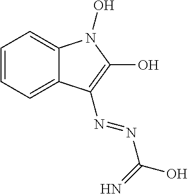 [SMILES: N=C(O)/N=N/c1c(O)n(O)c2ccccc12] | InChI = 1S/C9H8N 4O3/c10- 9(15)12-11-7-5- 3-1-2-4- 6(5)13(16)8(7) 14/h1- 4,14,16H,(H2,10, 15) |

The chemical compound may also be a derivative of a chemical compound listed in Table 1 or Table 2, such as compound 1 or compound 10 therein. Methods for derivatizing small-molecule organic compounds are well-known in the art. For example, Compound 10 is a hydrazone derived from isatin, and variants of this type of lead compound are easily accessible with simple condensation chemistry (Scheme 1):

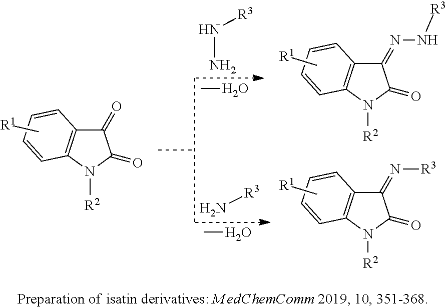

Notably, hydrazones are functional groups that are present in approved drugs (e.g., eltrombopag and edaravone), as well as numerous investigational and experimental drugs (e.g., levosimendan, talampanel, carbazochrome, ambazone). Importantly, an isatin-derived hydrazone is also known as an experimental drug (DrugBank: metisazone), a fact that supports further study of Compound 10 as a lead compound for development of a therapeutic targeting MYC in cancer.

### III. EXAMPLES

The following examples are provided for illustrative purposes only and are non-limiting.

### Example 1: Materials and Methods for the Indicated Experiments

### Cell Culture

HEK293T cells from ATCC® (CRL-3216™) were maintained in DMEM supplemented with 10% fetal bovine serum and prophylactic Plasmocin™ (InvivoGen) to prevent mycoplasma contamination. The HEK293T cell line is a highly transfectable derivative of human embryonic kidney 293 cells and contains the SV40 T-antigen. LookOut® Mycoplasma PCR Detection Kit (Millipore Sigma) was used to check for mycoplasma contamination every six months.

### Deletion Mapping Co-Immunoprecipitation

The indicated TRRAP constructs were cloned into a CMV-driven plasmid containing an N-terminal FLAG tag as previously described (23). Full-length wild-type (WT) MYC and MYC ΔMB2 (A129-145), and the indicated MYC constructs were cloned into the same CMV-driven plasmid but containing a Glu-Glu (PYO) tag instead. HEK293T cells were co-transfected with equal amounts of each plasmid using LipoD293™ In vitro DNA Transfection Reagent per protocol (SignaGen). Cells were plated subconfluently 16-20 hours prior to transfection. After 24 hours, cells were lysed in F-buffer (10 mM TRIS pH 7.5, 50 mM NaCl, 30 mM sodium pyrophosphate, 5 mM ZnCl2, 10% glycerol, 1% Triton-X, 50 mM NaF) supplemented with 1 mM PMSF, 10 μM Leupeptin, 10 μM Pepstatin-A, and 10 μg/mL Aprotinin for immunoprecipitations and co-immunoprecipitations. Immunoprecipitations were performed using anti-FLAG (Sigma Aldrich), anti-PYO (Covance), or anti-MYC (C33 Santa Cruz Biotechnology) agarose preconjugated beads. Co-immunoprecipitation was analyzed by western blots with the following antibodies: MYC (sc-764 Santa Cruz Biotechnology), TRRAP (A301-132A Bethel Laboratories), FLAG (F7425 Sigma Aldrich), and PYO (Covance).

### Protein Production and Purification

The indicted MYC or TRRAP constructs, or MYC-TRRAP fusions were cloned into a modified pGEX 6P-1 vector containing an additional C-terminal TwinStrep tag II (TS) from IBA Life Sciences. BL21 (DE3) E. coli were transformed with each of these vectors and stored at −802C in a 25% glycerol solution. A starter culture was prepared by adding a small amount of glycerol stock to 25 mL Terrific Broth (TB; BD Biosciences) with 50 μg/mL ampicillin. Next, this culture was incubated in a shaker overnight at 37° C./250 RPM and then divided into five 2 L flasks containing 500 mL of TB supplemented with ampicillin. After the OD of the culture reached 2.0, the flasks were placed in an ice bath until the temperature of the culture reached 16° C. Finally, Isopropyl 3-D-1-thiogalactopyranoside (IPTG) was added to a final concentration of 1 mM and the culture was incubated in a shaker at 16° C./250 RPM for 20-24 hours. The culture was subsequently centrifuged at 4° C., 6,000 RCF for 20 minutes and the pellet stored at −80° C. until purification.

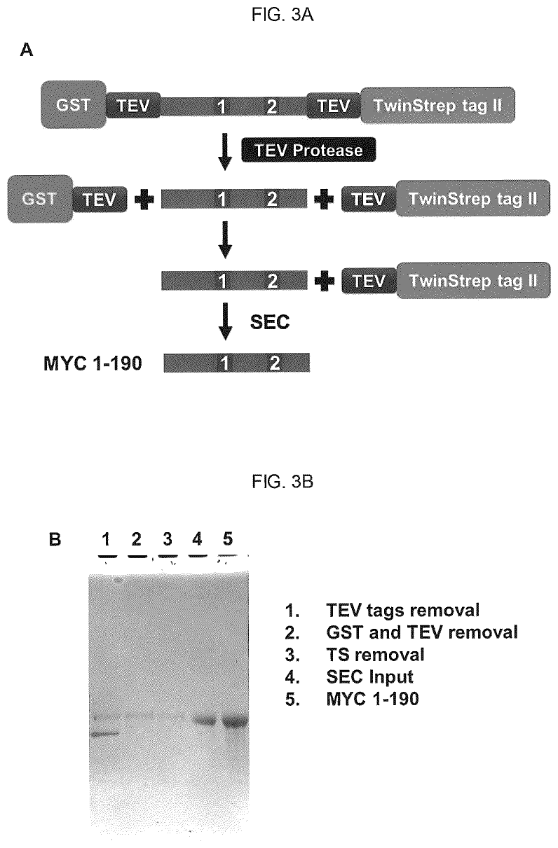

The frozen pellets were resuspended for lysis in 250 mL of a solubility-optimized buffer for MYC constructs containing: 100 mM TRIS, 150 mM NaCl, 5% Ethylene Glycol (EG), 1 mM EDTA, 1 mM TCEP, and 0.02% NaN3. Additionally, lysozyme was added at 1 mg/mL and protease inhibitors including: 1 mM PMSF, 10 μM Leupeptin, 10 μM Pepstatin A, and 10 μg/mL Aprotinin. The lysate was kept on ice for 30 min, sonicated at 70% amplitude with a Branson 250 sonicator for 3 min (10 sec ON, 10 sec OFF cycles), and spun >100,000 RCF for 60 min. The lysate was collected, and the pellet discarded. Using an NGC chromatography system (Bio-Rad), a 5 mL GSTrap (GE Healthcare) affinity column was used to purify the indicated GST fusion construct from the lysate. Following elution with the same lysis buffer minus lysozyme and protease inhibitors but supplemented with 20 mM reduced glutathione, the eluate was incubated overnight in the presence of HRV-3C protease (ThermoFisher) for the removal of the GST tag. Then, the products of this reaction were loaded onto a 5 mL StrepTactin XT® column (IBA Life Sciences) using the same chromatography system. After washing with the same buffer as above, constructs were eluted with 50 mM Biotin. This eluate was then incubated with Ac-TEV protease (ThermoFisher) for the removal of the TS tag. The products of this reaction were passed through a Ni-NTA gravity column (QIAGEN) for the removal of the Ac-TEV protease. The flow-through was concentrated and loaded on to a SEC Superdex 200 16/600 column (GE Healthcare) previously equilibrated with 1×PBS. Following elution, purity was confirmed using SDS-PAGE (FIG. 3B). Protein concentration was quantified using spectrometric analysis, and aliquots were flash-frozen in liquid N2 and stored at −80° C.

15N-labeled proteins were purified exactly as above. However, expression in E. coli differed. Starters were added to 250 mL of TB; the culture was incubated until an OD of 4.0 was reached. Then, the bacteria were centrifuged at 500 RCF for 20 min to remove the TB media. The pellet was then resuspended in minimal media (M9 media) containing 0.75 g 15NH4Cl and unlabeled dextrose. The culture was then incubated with 1 mM IPTG for protein induction and harvested as outlined above.

### Circular Dichroism Spectroscopy

The secondary structure of the indicated protein constructs (1 μM) was measured in 1×PBS with or without the indicated additives. CD spectra were acquired from 200 to 250 nm at 25° C. in a Jasco J-185 instrument using a 10 mm spectrosil cuvette (VWR). The mean residue ellipticity (MRE) was calculated using Equation 1:

$$[ θ ] = θ \frac{M}{10 L C} \qquad ( 1 )$$

where [θ] is the MRE, θ is the measured ellipticity in millidegrees, M is the average molecular weight in g/mol, L is the path length of the sample cell in centimeters, and C is the concentration of the protein in g/L.

### In-Vitro Pulldown

The specified purified MYC and C-terminal TS-tagged TRRAP protein constructs were mixed at 50 μM each and incubated at room temperature for 2 h in the presence of StrepTactin XT® beads in 1×PBS. After pulldown, bound proteins were eluted with 50 mM Biotin and analyzed with a Coomassie-stained SDS-PAGE. For MYC-TRRAP fusion proteins, the specified constructs were incubated with and without 30% ethylene glycol (EG) in 1×PBS for 30 min before linker cleavage with HRV-3C. Afterwards, the same pulldown and analysis followed.

### Size-Exclusion Chromatography in Ethylene Glycol

A Superdex 200 16/600 column (GE Healthcare) connected to an NGC chromatography system (Bio-Rad)_was used like above. The column was first equilibrated in 1×PBS supplemented with 30% EG. The indicated protein constructs were loaded onto the column and A280 spectra were collected in real-time. Due to a high system pressure, the flow rate for this method had to be reduced to 0.5 mL/min.

### NMR Spectroscopy

Both 1H measurements and 1H,15N-HSQC measurements were recorded at 25° C. with a 500 MHz Bruker NMR spectrometer equipped with a standard probe using 3 mm sample tubes. Unlabeled MYC 120-161 1H spectra were recorded in either 1×PBS or with 30% TFE-d2. 1H,15N-HSQC spectra of MYC 120-161 and MYC 120-161-TRRAP 2033-2088 were recorded in 1×PBS with 30% TFE-d2. Data were processed using TopSpin 4.0 (Bruker) and visualized using NMRFAM-SPARKY software (42).

### In-Cell PPI Luminescence Complementation

HeLa cells plated on Greiner 96-well TC-rated white plates with clear bottoms were used for the following measurements. Reverse transfections were carried out using LipoD293™ before cells were plated. A bluescript KS' plasmid (Addgene) was used as carrier DNA for transfections, and a pcDNA3.1 EGFP plasmid (ThermoFisher) was used as a fluorescence reporter. White light-reflecting film (USA Scientific) was used to cover the bottom of the plates for luminescence measurements. Black light-absorbing film was used to cover the top of the plates for fluorescence measurements. All measurements were taken on a SpectraMax i3 instrument (Molecular Devices).

First, the usable range of expression was determined where LgB and SmB complementation does not occur spontaneously. Transfections were carried out with increasing DNA amounts (1-100 ng per well) of a CMV-driven LgB plasmid and SmB in excess or vice-versa. Background luminesce for the usable range and the amount of DNA needed for vast excess of LgB or SmB were noted. Next, complementation with vast excess either LgB or SmB was used to determine the expression of each of the indicated MYC and TRRAP constructs for a range of DNA amounts per well: 10-100 ng. An optimal ratio of DNA to equalize the expression of each MYC and TRRAP pair was calculated. Then, signal-to-noise ratios were calculated for the indicated MYC and TRRAP pairs, and the pair with the highest ratio was chosen. Finally, the chosen pair was used to determine the optimal DNA transfection mixture. It was determined to be 6.7 ng of a plasmid with a MYC construct, 60 ng of a plasmid with a TRRAP construct and 33.3 ng of the pcDNA 3.1 EGFP plasmid, for each well in a 96-well plate. Unless otherwise indicated, these were the ratios of DNA transfected for this type of assay.

Using the optimal ratio described above, changes in TRRAP binding caused by point mutations to MYC 1-190 were measured using in-cell luminescence complementation. The indicated mutations were cloned into SmB-MYC 1-190 and transfected with TRRAP 2033-2283-LgB and EGFP into HeLa cells. Luminescence was measure as described above 48 h post-transfection.

### Screening NCI Small-Molecule Chemical Library Sets

HeLa cells were transfected as above using LipoD293™ with a mixture of SmB-MYC 1-190, TRRAP 2033-2283-LgB, and EGFP in CMV-driven plasmids at the same optimized ratio described above. Two days post-transfection, the media in each well was replaced with fresh media containing each compound from the NCI's sets at 25 μM. Cells were incubated for 2 h with each compound, and luminescence and fluorescence measurements were recorded for each well. Changes in luminescence were normalized to fluorescence. The following pre-plated compound sets were obtained from the NCI/DTP chemical repository and used for this screen:

### Approved Oncology Drugs Set VIII:

The compounds in the Approved Oncology Drugs Set VIII were delivered in Greiner 650201 96-well PP U-bottom plates. Each well contained 20 μL of a compound at 10 mM in DMSO. All proprietary agents in this set were obtained through commercial sources. All compounds were found to have satisfactory purity and identity.

### Diversity Set VI:

The compounds in the NCI's Diversity Set VI were delivered in Greiner 650201 96-well PP U-bottom plates. Each well contained 20 μL of a compound at 10 mM in DMSO. All compounds were checked for purity via LC/Mass Spectrometry and found to have a purity of 90% or better.

### Mechanistic Set IV:

The compounds in the Mechanistic Set IV were delivered in Greiner 650201 96-well PP U-bottom plates. Each well contained 20 μL of a compound at 1 mM in DMSO.

### Natural Products Set IV:

The compounds in the Natural Products Set IV were arrayed across two 384-well polypropylene (PP) microtiter plates. Each well contained 0.20 μmol of compound plus 1 μL of glycerol; 20 μL of a 10 mM solution of each compound was obtained by the addition of 19 μL of DMSO to each well.

### Statistics

All experiments were repeated at least 3 times. An unpaired student's t-test was performed to determine standard deviation and statistical significance. P-value 0.05 was considered statistically significant. Error bars represent SEM.

### Example 2: Mapping the MYC:TRRAP Interaction

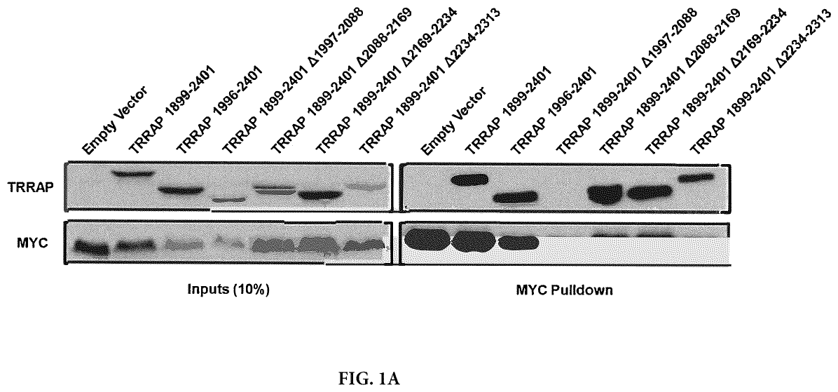

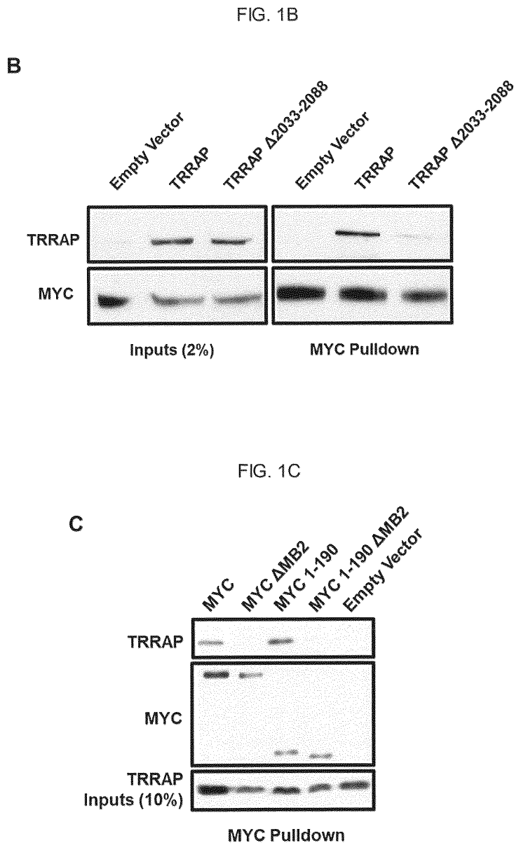

Mapping of the MYC:TRRAP interaction was initiated with a series of external and internal deletions within residues 1899-2401 of TRRAP (39). These deletions were constructed using proline residues as boundaries which largely correspond to the HEAT repeat boundaries (41). Through a series of co-immunoprecipitation experiments, the most critical MYC-interacting region in TRRAP was determined to be within residues 1997-2088, without which the TRRAP:MYC interaction cannot occur in transient assays (FIG. 1A). Although transient expression of the MYC protein and these TRRAP constructs vary significantly, it is still clear that the construct that lacks residues 1997-2088 of TRRAP is the most defective MYC binder. These TRRAP constructs were aligned with the results described by Knutson and Hahn and Diaz-Santin et al. (40, 41). Structural predictions for the most critical region suggest that it is inherently disordered, unlike its flanking domains.

To validate the mapping data above, a similar domain dependence was studied with full length proteins. An expression construct for full length TRRAP (1-3830) was created, plus a similar construct lacking only the predicted intrinsically disordered domain (amino acids 2033-2088). Thus, the latter lacks only 55 amino acids out of the native 3830 amino acids in TRRAP. FIG. 1B shows this small deletion mutant is defective for interaction with full length MYC. In this experiment the transient expression is consistent for each construct used. Thus, the intrinsically disordered region in TRRAP (2033-2088) is necessary for MYC interaction. The identification of a clear region that is necessary for the MYC:TRRAP interaction in human cells is inconsistent with the conclusions drawn from previously published mapping studies (39). The data presented here suggests that solubility and/or conformational differences exist within this region of TRRAP when produced in bacteria, which can be the cause of the inconsistency between these two mapping studies.

Similar mapping studies were conducted on MYC (1-439) to define its domain of interaction with TRRAP. Stable binding appears to require amino acids 1-190 of MYC, which encompass a large portion of the TAD. Importantly, an internal deletion of MB2 (17 amino acids) within this domain largely eliminates TRRAP binding, consistent with earlier studies (FIG. 1C) (23). The relatively large domains in both TRRAP and MYC required for a stable interaction may suggest an extended protein-protein interface, although large domains may also be required to ensure proper folding of a small protein-protein interface. The fact that small deletions within each domain can eliminate binding (i.e., TRRAP 2033-2088 and MYC MB2) indicates these small regions may be the most crucial sites for binding and, therefore, small molecule targeting.

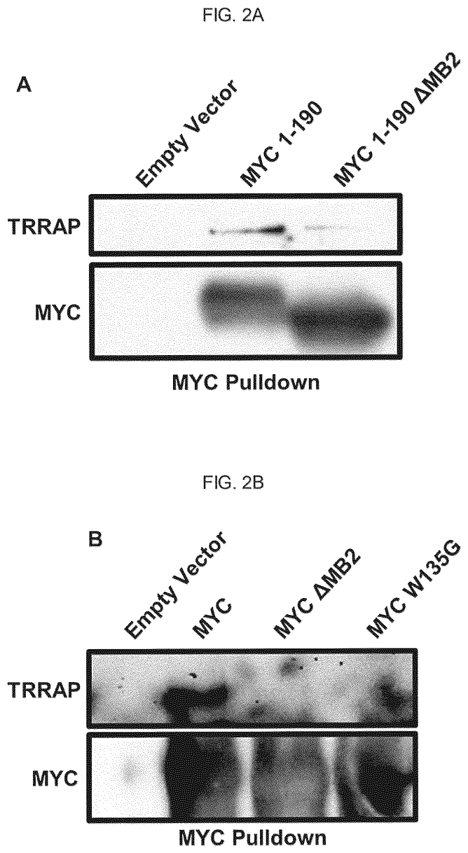

To validate the MYC's minimal domain of interaction (i.e., MYC 1-190), a co-IP experiment was performed, testing the binding of this domain to endogenous TRRAP. MYC 1-190 co-IPs with endogenous TRRAP, and this interaction requires MB2 (FIG. 2A). Finally, another co-IP experiment was performed to test the importance of the MB2 W135 amino acid (FIG. 2B) which is essential for MYC-driven cellular transformation (21, 23). The results indicate that W135 of MYC is indispensable for the co-IP of the complex.

### Example 3: Secondary Structure of MYC and TRRAP

To gain further insight into the secondary structure of MYC:TRRAP, we produced pure protein constructs in large quantities in E. coli (FIG. 3A). This involved sequential affinity purification using a GST N-terminal tag and a C-terminal TS tag. Upon tag removal, size exclusion chromatography (SEC) was used to assess the monomeric state of protein constructs and to buffer exchange. This resulted in very pure and highly concentrated protein constructs meeting the requirements for structural determination experiments (FIG. 3B).

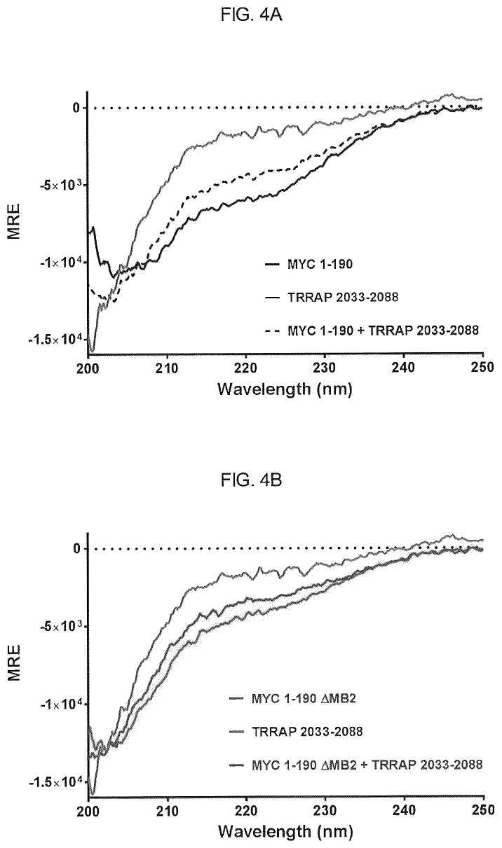

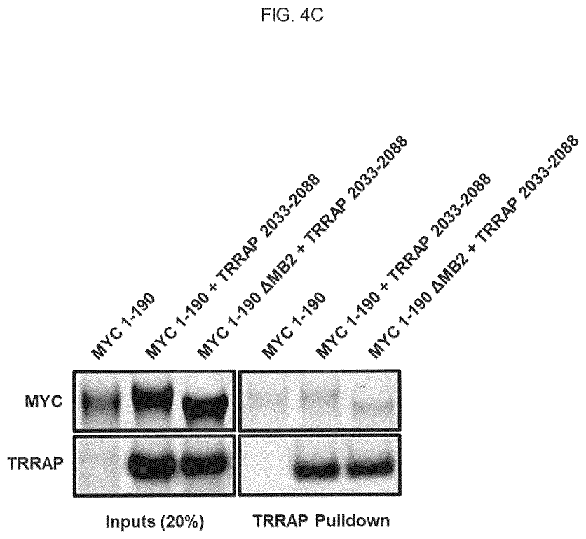

The secondary structures of the MYC TAD and TRRAP 2033-2088 were evaluated by CD spectroscopy (FIG. 4A). Although TRRAP 2033-2088 was suspected to be intrinsically disordered, nothing of its actual structural conformation was known (41). CD measurements revealed that this domain of TRRAP is, in fact, an IDR, lacking any measurable α-helical or 3-sheet secondary structure. The MYC TAD has also been described as an IDR, but the largest domain ever studied was MYC 1-143 (43). CD spectra of MYC 1-190 confirms that the MYC TAD is largely disordered but with some helical characteristics, consistent with previous findings (43, 44). However, the deletion of MB2 removes the minimum at 222 nm while conserving the minimum at 208 nm (FIG. 4B). This suggests that MB2 contains some of the α-helical elements attributed to the MYC TAD. With the hypothesis that MYC and TRRAP acquire a stable conformation upon binding, CD was used to test whether there was any gain in newly acquired secondary structure upon mixing MYC 1-190 and TRRAP 2033-2088. FIG. 4B shows that there was no gain in secondary structure when these two constructs were mixed in vitro at a 1:1 molar ratio. There was no change in measurements at concentrations between 1-10 μM. Finally, a co-IP experiment was performed with purified proteins to determine whether MYC 1-190 and MYC 1-190 ΔMB2 exhibited any difference in binding to TRRAP 2033-282 2088. The proteins were mixed at a 1:1 molar ratio (50 μM each). Binding was assayed by Coomassie-stained SDS-PAGE (FIG. 4C). There was no difference of TRRAP 2033-2088 binding to either MYC construct, suggesting that MYC 1-190 does not form a complex in vitro.

### Example 4: Induction of an Ordered Structure on MYC:TRRAP

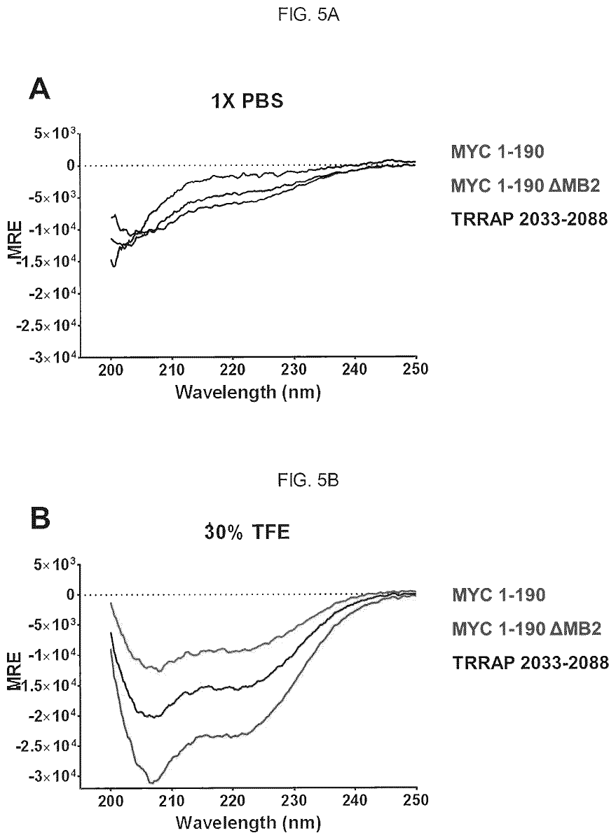

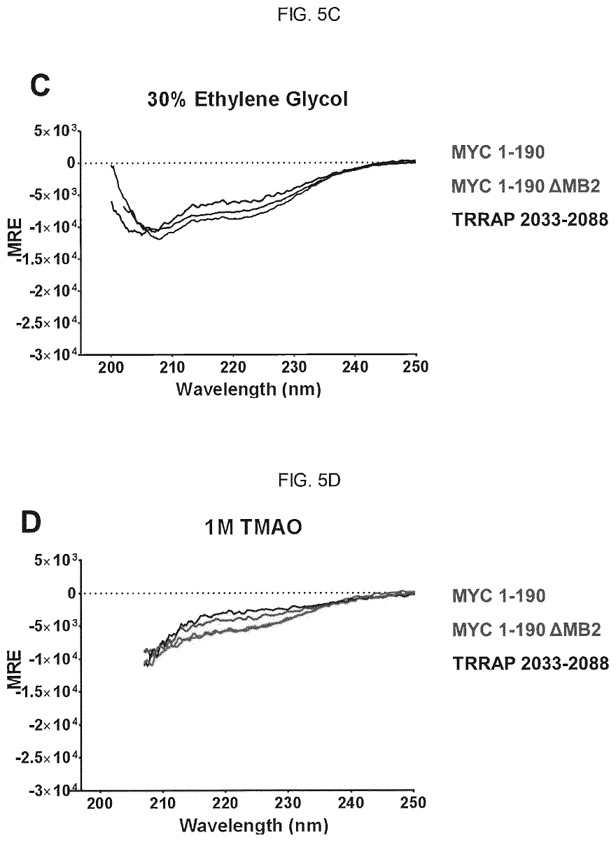

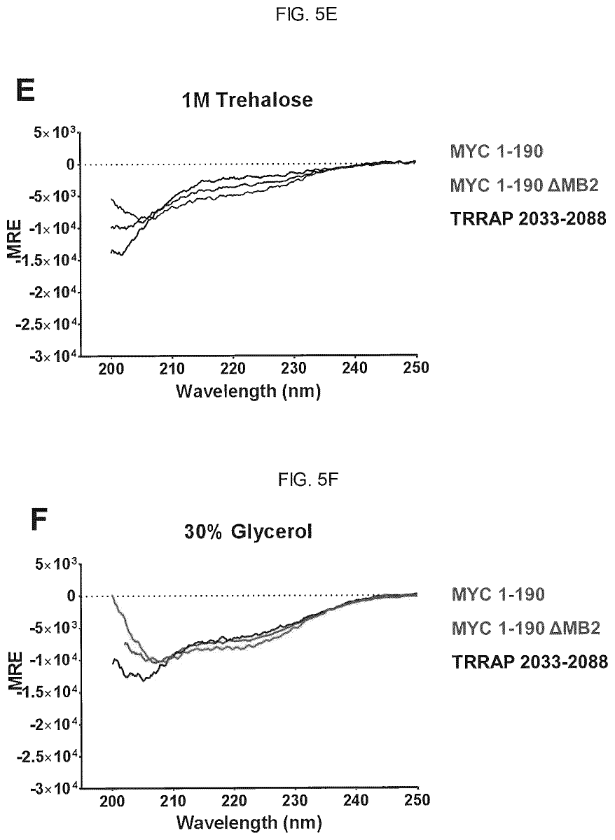

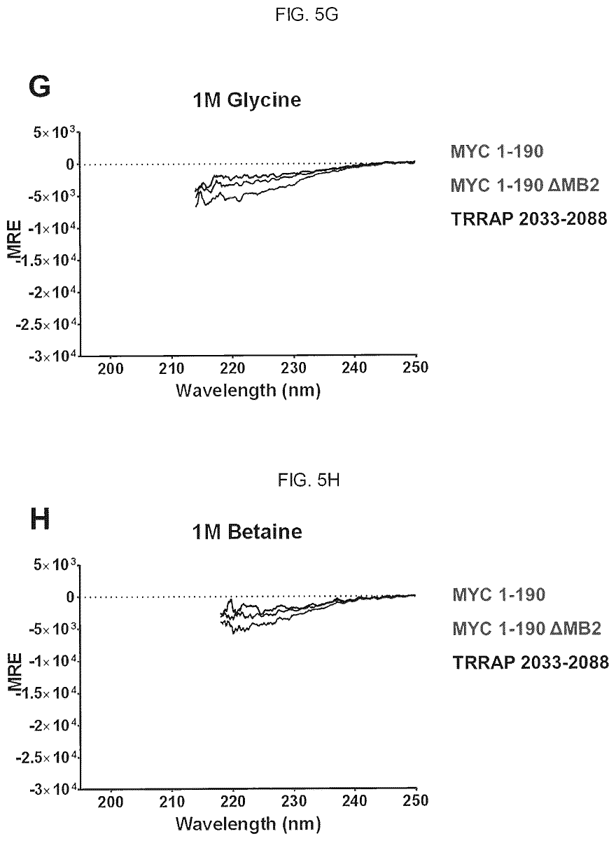

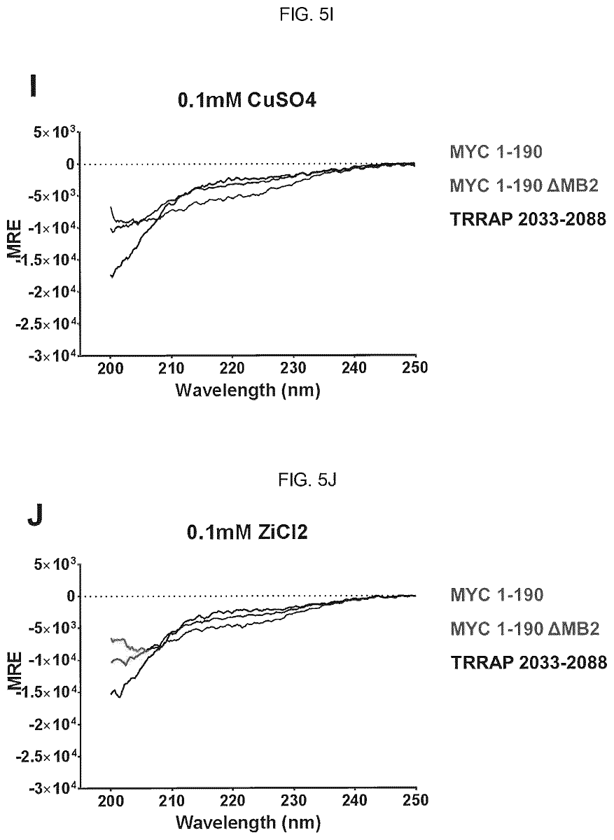

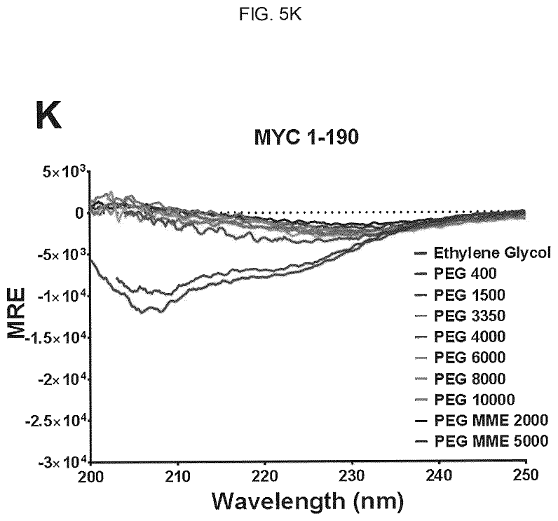

We explored alternative conditions to aid the formation of a protein complex in vitro. Although the regions of interaction of both MYC and TRRAP are IDRs, an ordered conformation could occur upon dimerization. Different methods of stabilizing an interaction have been described in the literature. Two precedents are the MYC:MAX crystal structure and the more recent NMR structure of the p53 TAD, both of which created protein-protein complexes from primary fusion constructs (11, 45). Furthermore, we tested additives or molecular chaperones that could induce secondary structure in MYC, TRRAP, and/or a MYC:TRRAP complex. To test different molecular chaperones, the secondary structure of each construct was characterized by CD spectroscopy in the presence of additives. These constructs included: MYC 1-190, TRRAP 2033-2088, and MYC 1-190 mixed in vitro with TRRAP 2033-2088 (FIG. 5A-FIG. 5K). The additives tested included mostly osmolytes, along with some metal ions and organic solvents. Table 2 summarizes the results from these measurements.

TABLE 3
The effect of additives on MYC: TRRAP

| Additive | Secondary Structure |
|---|---|
| PBS | Unstructured |
| Glycerol | Partially α-helical |
| Ethylene Glycol | α-helical |
| Trehalose | Unstructured |
| Glycine | Unstructured |
| Betaine | Unstructured |
| Trimethylamine N-oxide | Unstructured |
| PEG400 | Partially α-helical |
| PEG1500 | Unstructured |
| PEG3350 | Unstructured |
| PEG4000 | Unstructured |
| PEG6000 | Unstructured |
| PEG8000 | Unstructured |
| PEG 10000 | Unstructured |
| PEG MME 2000 | Unstructured |
| PEG MME 5000 | Unstructured |
| ZiCl2 | Unstructured |
| CuSO4 | Unstructured |
| 2,2,2-Trifluoroethanol | Highly α-helical |
| Methanol | Unstructured |
| Ethanol | Unstructured |

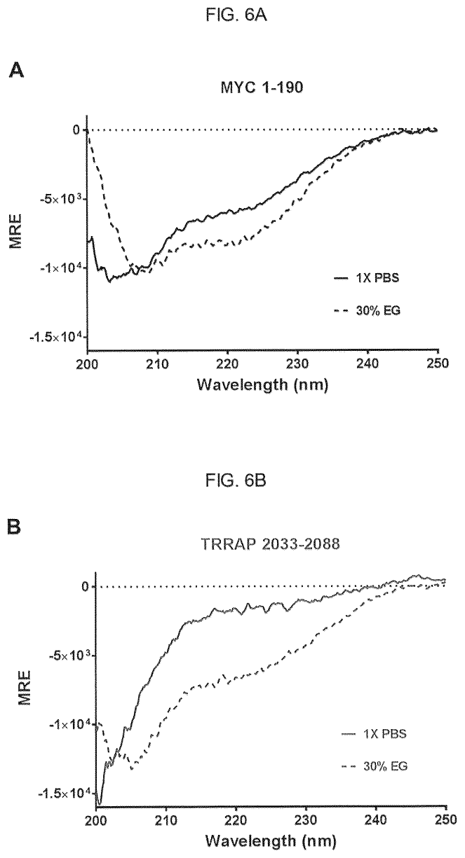

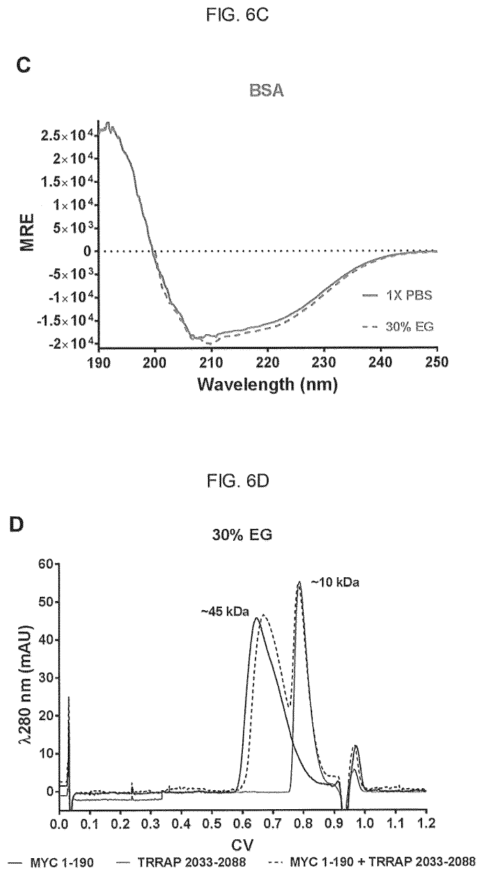

Of the additives tested, ethylene glycol (EG) and 2,2,2-Trifluoroethanol (TFE) produced the most specific effect and the largest gain in secondary structure, respectively. EG induces a secondary structural change in both MYC and TRRAP, but not BSA (FIG. 6A-FIG. 6C). To test whether EG could induce a MYC:TRRAP complex, samples containing MYC 1-190, TRRAP 2033-2088, and MYC 1-190 mixed with TRRAP 2033-2088 (100 μM each) in 30% EG were run on an SEC column equilibrated with 30% EG (FIG. 6D). Only two peaks were observed using the mixed sample, confirming that EG does not induce a MYC:TRRAP complex.

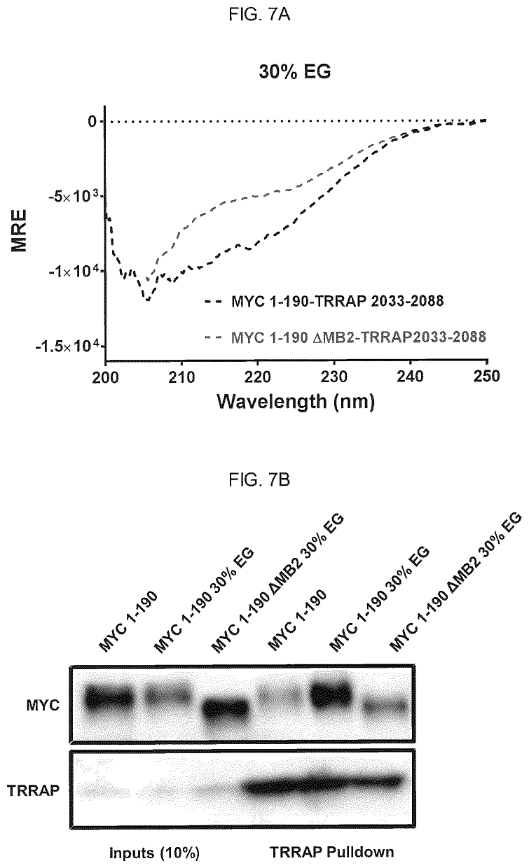

The MYC-MAX and p53-CBP structures suggest that complexes of two IDRs can be established using a covalent linker. Therefore, expression of the minimal-interacting regions of TRRAP and MYC were produced as a fusion protein separated by a computationally-designed flexible linker (GSGSAGSAAGSGEFG) (reviewed in 46). The effects of EG on a MYC-TRRAP fusion protein were compared to MYC ΔMB2-TRRAP using CD spectroscopy (FIG. 7A). EG had a more profound effect on the secondary structure of the fusion protein containing MB2. This indicates that a fusion protein may be required to form a stable MB2-dependent MYC:TRRAP complex in vitro. To test whether a complex is being formed, two fusion proteins were produced with a cleavable 3C protease site between the MYC and TRRAP domains, MYC 1-190-TRRAP 2033-2088-TS and MYC 1-190 ΔMB2-TRRAP2033-337 2088-TS. After the addition of EG, the linker was cleaved, and any potential complex assessed by a pulldown experiment followed by Coomassie-stained SDS-PAGE (FIG. 7B). These results suggest that a MYC:TRRAP complex was formed only in the presence of EG and that the complex remained bound after the cleavage of the linker and the removal of EG. Furthermore, the complex requires MB2, consistent with the complex that forms in vivo. These results point to a native-like complex, formed in vitro with the aid of a flexible linker and stabilizing additives.

### Example 5: ¹H, ¹⁵N-HSQC Spectrum of MYC Vs MYC-TRRAP

Because W135 of MYC is critical for cellular transformation and for the MYC:TRRAP interaction, an 1H, 15N-HSQC of MYC with W135 assigned would be extremely useful for screening inhibitors of MYC activity in cancer. The indole N—H pair in a tryptophan side-chain gives its chemical shift peak in the HSQC spectra a unique and distinctive appearance. Therefore, an HSQC spectrum of MYC could have the W135 side-chain N—H pair assigned without necessarily assigning all other peaks.

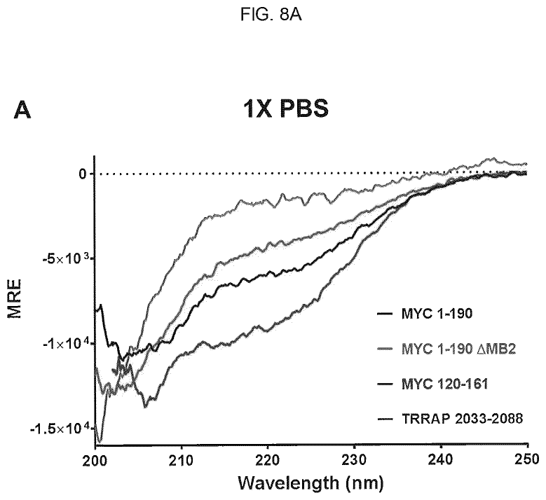

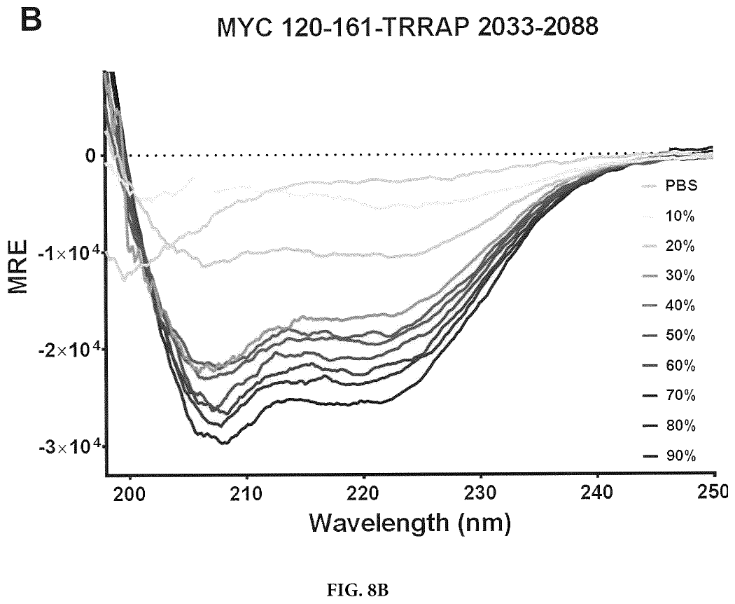

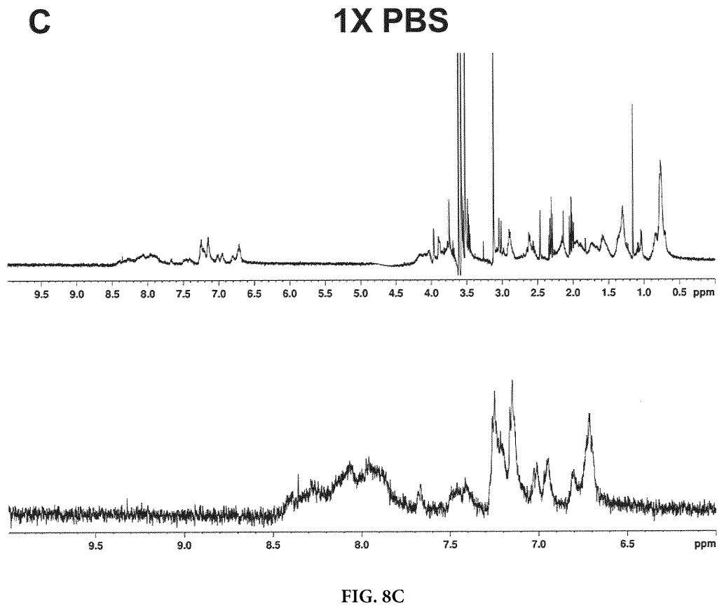

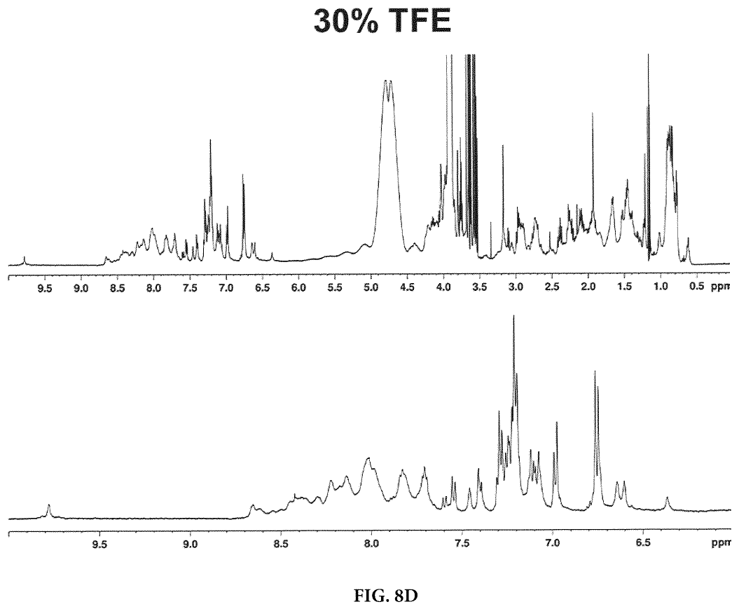

Since TFE induced the highest gain in secondary structure in MYC measured by CD (Table 2), NMR experiments were carried out to characterize the structural elements of MYC 120-161 and MYC 120-161-TRRAP 2033-2088 in the presence of TFE-d2. These constructs were chosen because W135 of MYC is the only tryptophan residue within this segment, and this region of the MYC TAD has the most stable secondary structural elements, even in PBS (FIG. 8A). Therefore, CD measurements of MYC 120-161-TRRAP 2033-2088 were taken with increasing TFE concentrations from 10%-90% (v/v) (FIG. 8B). These measurements show that, in the presence of 0-20% TFE, the resulting complex is too unstructured to warrant further measurements. However, the complex showed highly α-helical character in the presence of 20-30% TFE. There were minimal gains in secondary structure upon increasing the TFE concentration beyond 30%.

Before HSQC measurements were carried out, simple one-dimensional 1H-NMR spectra were collected to confirm that MYC 120-161 had a measurable W135 signal in the presence of 30% (v/v) TFE-d2. As shown in FIG. 8C-FIG. 8D, a peak in the chemical shift (~9.8 ppm) consistent with that of a tryptophan residue side-chain appears only in the presence of TFE. Additionally, the dispersion of peaks between 6-10 ppm in TFE (FIG. 8D) compared to PBS (FIG. 8C) demonstrates that MYC 120-161 transitions from an unfolded to a folded state.

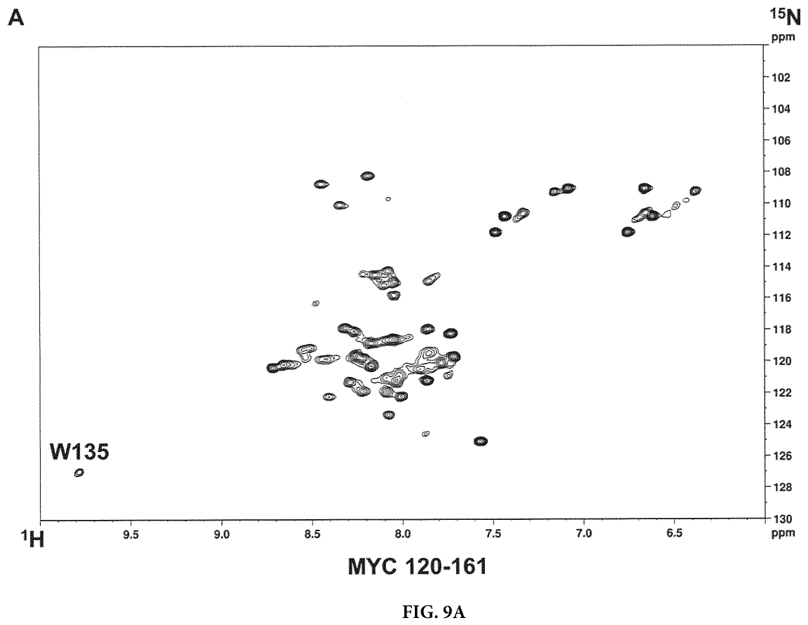

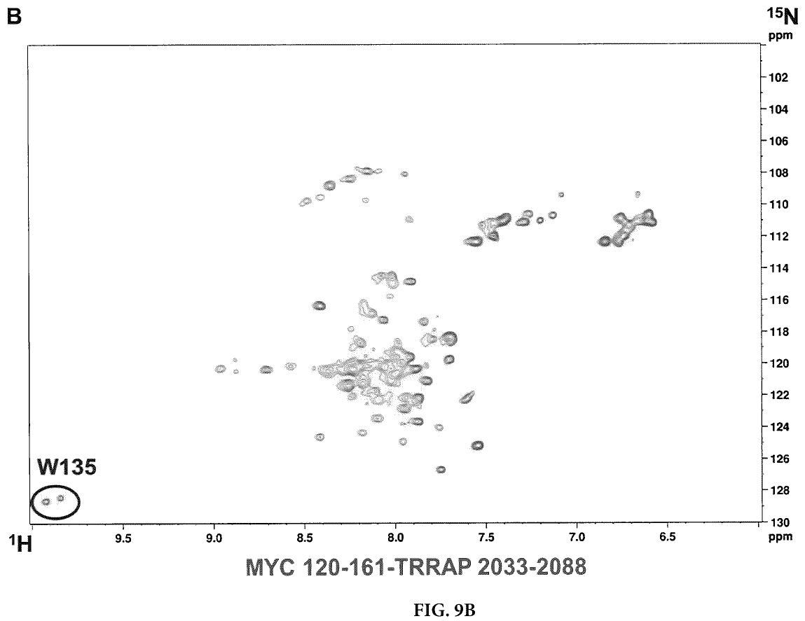

Next, HSQC measurements of 15N MYC 120-161 and MYC 120-161-TRRAP 2033-2088 were compared to perform the assignment of W135 and determine if a binding event can occur (FIG. 9A-FIG. 9B). In the MYC alone construct containing 54 residues, 65 peaks were resolved using NMRFAM-SPARKY's automated peak picking (APES) utility (47). It contained 1 P, 1 R, 2 Ns, 3 Qs, 1 W, and no H residues. The 5 Ns and Qs side-chain N—H pairs match exactly to the predicted peaks in the 108-112 ppm 15N and 6-7.5 ppm 1H region. These peaks are doublet proton peaks with the same nitrogen chemical shift. The W135 N—H side-chain pair has a nitrogen chemical shift of ~127 ppm and a hydrogen chemical shift of ~9.8 ppm, typical of a N—H indole pair (FIG. 9A).

In the 127 residue MYC-TRRAP construct, 122 peaks were resolved as above (FIG. 9B). However, this construct contained 3 Ps, 6 Rs, 4 Ns, 5 Qs, 1 W, and no H residues. More importantly, the W135 N—H side-chain pair had a split peak resonance, suggesting that it resides in two different environments and hence there are likely two different conformations for the construct. Considering both spectra, one of the two chemical shifts of the split peak can represent each of the two conformations. Since TRRAP binding requires W135 (FIG. 2B), these two conformations provide evidence for the existence of a bound and an unbound MYC:TRRAP state in TFE. The ratio of intensities of these two peaks is an indicator of the ratio of bound and unbound complexes. These measurements validated the previous data and confirm that a stable 3D structure of MYC:TRRAP can exist in the presence of TFE. The assignment of W135's chemical shift peak in the context of both MYC 120-161 and MYC 120-161-TRRAP 2033-2088 indicates its usefulness for detecting the MYC:TRRAP binding interaction and for determining a reduction or an absence of this interaction in ligand screening assays to identify inhibitors of a binding interaction between MYC and TRRAP.

### Example 6: In-Cell Luminescence Complementation Assay

Deletion mapping enabled the identification of the minimal interacting domains of MYC and TRRAP. By reducing the size of the binding complex, it is now possible to accurately assay small-molecule interactions. To accomplish this, an in-cell luminescence complementation assay was used, namely a split luciferase system called NanoLuc® Binary Technology (NanoBiT®), developed by Promega Corporation. This assay was established using a novel 19.1 kDa, monomeric, highly soluble and stable, ATP-independent luciferase enzyme called NanoLuc® (65). The NanoLuc® enzyme was split into two parts: Large BiT (LgB; 18 kDa) and Small BiT (SmB; 11 amino acid peptide). These are used as tags on the two proteins of interest; upon protein dimerization, the tags complement and form a highly active luciferase enzyme.

Using the minimal domains that form the MYC and TRRAP complex, each grafted to the LgB and SmB tags, an in-cell luminescence complementation system was developed that can be used to measure direct binding interactions of MYC and TRRAP mutants or the inhibition of binding by small-molecules. The orientation of the tag-binding domain complexes and the levels of protein expression were optimized before luminescence measurements were taken. These measurements revealed novel aspects of the interaction and of MYC biology in cancer. Later, the assay was adapted for a screen of small-molecule inhibitors of the MYC:TRRAP interaction. Several compound libraries were received from the NCI/DTP Open Chemical Repository (http://dtp.cancer.gov). The results and details of this screen are discussed further in Example 7.

Establishing an in-Cell Luminescence Complementation Assay

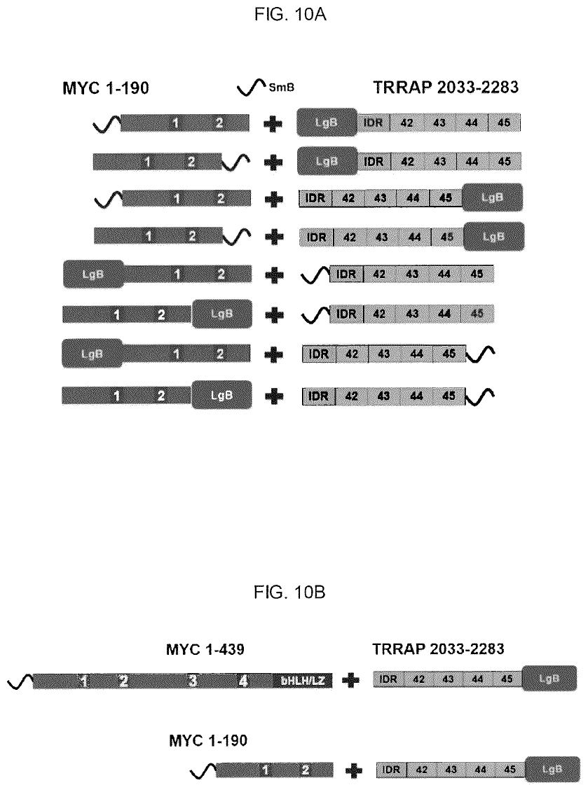

With high sensitivity and broad dynamic range, bioluminescent methods have proven useful for many applications, including binding assays and drug discovery. Native enzymes and substrates have been incrementally adapted to existing methodologies to great advantage. Using directed evolution from a deep-sea shrimp luciferase, Oplophorus gracilirostris, Promega Corporation engineered a novel bioluminescence system (65). The resulting NanoLuc® enzyme is a 19.1 kDa protein that produces a glow-type luminescence (half-life >2 h) when the novel substrate, furimazine, is added. Further investigation resulted in the creation of NanoBiT®, a split version of this system intended for measurement of PPIs in live cells. Unlike co-IPs and other binding assays, the NanoBiT® system enables quantifiable measurements without cell lysis. Specifically, live cells were transiently transfected to express two vectors: one containing MYC with a luminescence tag; the other containing TRRAP with a complementary tag. Luminescence was observed upon complementation of the NanoLuc® enzyme only in the presence of a MYC:TRRAP interaction. LgB and SmB tags have a low affinity for each other; only by bridging them in proximity can complementation occur. To prevent nonspecific association of the NanoBiT tags and to ensure that only a specific and direct interaction of MYC and TRRAP would result in luminescence, only low levels of expression should be used in this type of assay. Therefore, an appropriate expression vector and mammalian promoter had to be selected. Per the manufacturer's recommendations, full-length MYC and MYC 1-190, and TRRAP 2033-2283 and TRRAP 2033-2088 were each cloned into four mammalian expression vectors containing a Herpes Simplex Virus-1 Thymidine Kinase (HSV-TK) promoter. Full-length TRRAP was cloned into two vectors only containing an N-terminus tag of either LgB or SmB. HSV-TK is a low-expressing constitutive promoter with expression levels as low as ^{~}100-fold compared to CMV-driven vectors. Each of the four vectors had an N-terminus LgB or SmB tag, or a C-terminus LgB or SmB tag. It is necessary to optimize the orientations of the tags to optimize the signal-to-noise ratio of the assay. FIG. 10A summarizes all eight possible combinations of MYC and TRRAP pairs with the LgB and SmB tags.

None of these construct pairs in a vector with an HSV-TK promoter produced detectable luminesce at 48 h post-transfection. We reasoned that a higher expressing promoter was necessary. Therefore, all constructs, including tags, were moved into a CMV-driven mammalian expression vector. Luminescence was detectable using this expression system. However, variability in transfection efficiency and cell number had to be controlled. To do so, a pcDNA3.1 plasmid containing EGFP was co-transfected with all LgB and SmB pairs. Fluorescence measurements were taken immediately after every luminescence measurement and used for normalization.

During initial luminescence measurements, it became clear that most constructs had varying levels of protein expression, and this variation was especially noticeable given their low levels of expression. Therefore, it was necessary to measure the differential protein expression levels of each of the MYC and TRRAP constructs. Unfortunately, expression levels were too low for western blotting or in-cell western assays. The amount of expression required to obtain a reliable signal in any of these methods was past the saturation point for the luminescence assay. Taking measurements outside of the range of the luminescence assay proved that the differential in expression levels between constructs observed in any overexpressed system did not correlate to those values in a low-expression assay. Consequently, a method of measuring low protein expression levels was needed.

Ideally, the same luminescence system could measure protein levels and binding of the MYC and TRRAP constructs. We determined that excess LgB or SmB could complement with low expression MYC or TRRAP LgB/SmB fusion protein to give a quantifiable luminescence signal indicative of the construct's relative level of expression. Since SmB is too small to express on its own, a fusion of Halo tag-SmB was obtained from Promega. Overexpressing either LgB or Halo-SmB in the presence of any of the complementary fusion constructs allowed the quantification of fusion construct expression. This allowed DNA transfection protocols to be adjusted to equalize cellular expression levels.

With cells now expressing each construct in equal amounts, signal-to-noise ratios were determined in each MYC and TRRAP pair. The pairs that were chosen as a result are shown in FIG. 10B. Fortunately, the same vector pair was optimal for MYC 1-190 with TRRAP 2033-2283 and full-length MYC with TRRAP 2033-2283, enabling direct comparison. The tagged N-terminal region is the same in full-length MYC and MYC 1-190. The same is true of TRRAP's C-terminal region, simplifying comparison of the two pairs.

Two additional TRRAP constructs, full-length TRAAP and amino acids 2033-2088, did not produce measurable luminescence when co-transfected with MYC full-length or MYC 1-190. TRRAP 2033-2088 did not show any binding when transiently transfected and co-IPed either; perhaps this region of TRRAP is necessary but not sufficient for MYC binding. Full-length TRRAP, on the other hand, has been shown to co-IP with full-length MYC and MYC 1-190. However, LgB/SmB tags require the use of an optimized 15 residue linker. The N-terminus of TRRAP may be far enough from the MYC interacting domain that complementation of the luciferase enzyme would require a much longer linker region.

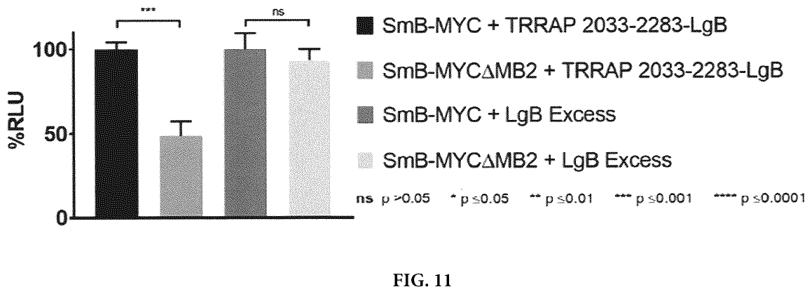

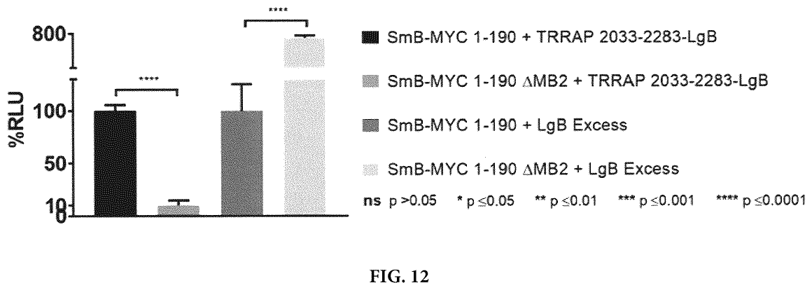

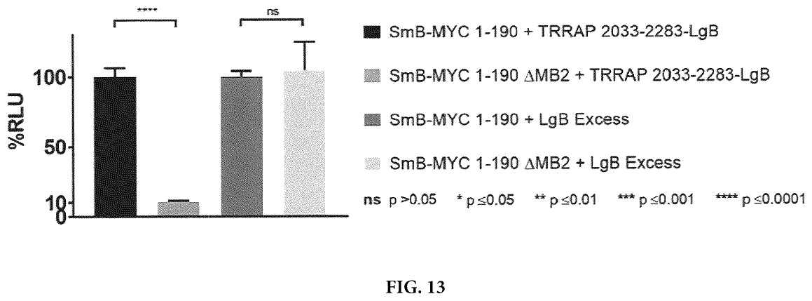

After obtaining reproducible luminescence complementation measurements with TRRAP 2033-2283 co-transfected with either full-length MYC or MYC 1-190, the protocol was repeated with MB2 removed from the respective MYC constructs. FIG. 11, FIG. 12, and FIG. 13 show the result of these experiments. For both MYC and MYC 1-190, binding to TRRAP is MB2 dependent. MYC 1-190 shows more dependence on MB2, perhaps because it lacks residues involved in secondary contacts. However, directly comparing MYC and MYC 1-190 shows the same level of luminescence, which is consistent with findings from the co-IP experiments presented in Example 2.

There was no measurable difference in expression between MYC, MYC ΔMB2, and MYC 1-190. However, MYC 1-190 ΔMB2 expression was higher than the rest of the constructs (FIG. 12). Its transfection protocol was adjusted until MYC 1-190 ΔMB2 expressed the same amount of protein as MYC 1-190 (FIG. 13). Given its greater dependence on MB2, the MYC 1-190 and TRRAP 2033-2283 pair were chosen for further experimentation, namely investigation into point mutations and small-molecule inhibitors.

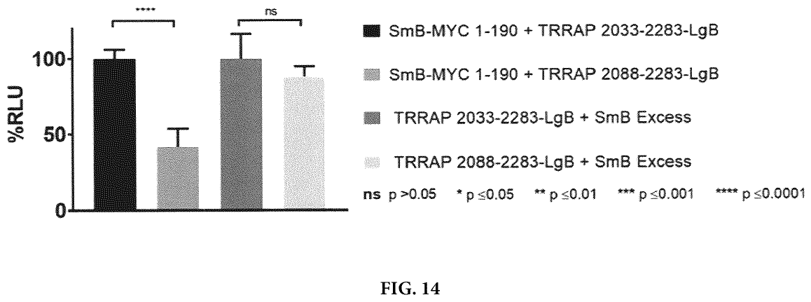

First, MYC:TRRAP's dependence on TRRAP 2033-2088 had to be confirmed considering the failure of the TRRAP 2033-2088 construct to produce luminescence complementation. In vivo binding measurements of TRRAP 2033-2283 were compared to a similar construct lacking the MYC binding region, TRRAP 2088-2283 (FIG. 14). Like MB2, the absence of TRRAP 2033-2088 diminishes binding, consistent with co-IP experiments. It is worth noting that expression of both TRRAP constructs was significantly lower than that of the MYC constructs—900% more DNA was transfected to produce a similar level of expression.

These experiments confirm that luciferase assays can be used to assess differential changes in MYC:TRRAP binding. Therefore, they can also be used to test small-molecule chemical libraries and identify inhibitors of the MYC:TRRAP interaction.

### Measuring Key Factors of the MYC:TRRAP Interaction

A 10-fold difference in luminescence complementation was observed when TRRAP 2033-2283 was co-transfected with MYC 1-190 versus MYC 1-190 ΔMB2. Given the magnitude of this difference, very small changes in affinity, arising from point mutations, can be detected with high sensitivity. Although co-IP experiments are ineffective for this application, the broad dynamic range of bioluminescence make it an appropriate assay.

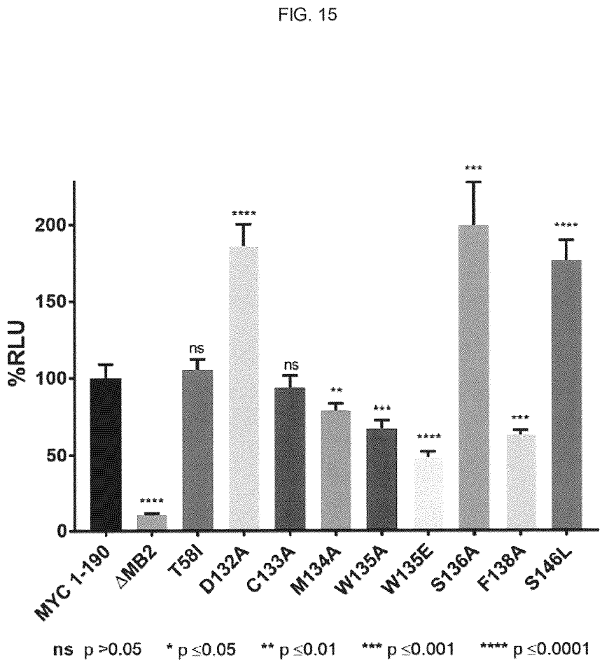

A series of point mutations were created in MYC 1-190, and any changes in TRRAP 2033-2283 binding were measured via luminescence complementation. Key residues were substituted with alanine residues (D132, C133, M134, W135, S136, and F138) or glutamate (W135). Additionally, two of the most common MYC mutations in cancer (T581/A/P/N and S146L) were screened (66, 67). Fluorescence by EGFP was used to normalize luminescence measurements by correcting protein expression levels (FIG. 15). The expression level for each construct was determined with the previously described luminescence-based assay.

Substitutions of major conserved MB2 residues (D132A, C133A, M134A, W135A, S136A, F138A, and W135E) confirmed their relative importance in the MYC:TRRAP interaction. A decrease in luminescence complementation is indicative of a residue that may participate directly in contacts between MYC and TRRAP. W135 proved essential once more, both in the case of W135A and W135E. M134A also caused a significant decrease in luminescence complementation, though not as much as W135A/E. C133A did not appear to affect binding. A novel finding, F138A showed the same decrease in luminescence as W135A. This suggests that F138 may have a meaningful participation in the MYC:TRRAP interaction. Quite unexpectedly, D132A and S136A produced a significant increase in luminescence, suggesting an increase in the affinity of MYC:TRRAP.

Two of the most common and recurrent MYC mutations in cancer, T581/A/P/N and S146L, were tested using the same in-cell luminescence complementation assay. FIG. 15 shows that T581 produced no change in TRRAP binding, despite a significant increase in expression. S146L produced a significant increase in luminescence complementation, suggesting an increase in TRRAP binding. These data may indicate a link between a previously undescribed gain-of-function mutation and MYC:TRRAP binding, the first reported link of its kind.

### Example 7: Screening Small-Molecule NCI Chemical Libraries in the in-Cell Luminescence Complementation Assay

The goal of developing an in-cell MYC and TRRAP PPI luminesce assay was to create a primary screen for use in drug discovery. For this purpose, four small-molecule chemical libraries were requested from the NCI/DTP Open Chemical Repository. These are listed below:

### Approved Oncology Drugs Set VIII:

### A Set of FDA-Approved Anticancer Drugs Consisting of 133 Agents

### Diversity Set VI:

The Diversity Set VI Consists of 1584 Compounds Derived from 140,000 Compounds Using the programs Chem-X (Oxford Molecular Group) and Catalyst (Accelrys, Inc.). These programs use defined pharmacophoric centers and defined distance intervals to create a finite set of three dimensional, 3-point pharmacophores resulting in over 1,000,000 possible pharmacophores.

### Mechanistic Set IV:

The Mechanistic Set IV consists of 811 compounds derived from 37,836 compounds that have been tested in the NCI human tumor 60 cell line screen. This mechanistic diversity set was chosen to represent a broad range of growth inhibition patterns.

### Natural Products Set IV:

The Natural Products Set IV consists of 419 compounds selected by origin, purity, structural diversity, and availability of compound.

These chemical sets were used to discover novel small-molecule inhibitors of the MYC:TRRAP complex. SmB-MYC 1-190, TRRAP 2033-2283-LgB, and EGFP were transfected into HeLa cells. Two days post-transfection, compounds were added to the media at 25 μM and cells were incubated for 2h. Luminescence and fluorescence were recorded for each well containing one of the compounds. Changes in luminescence measurements were normalized to fluorescence measurements.

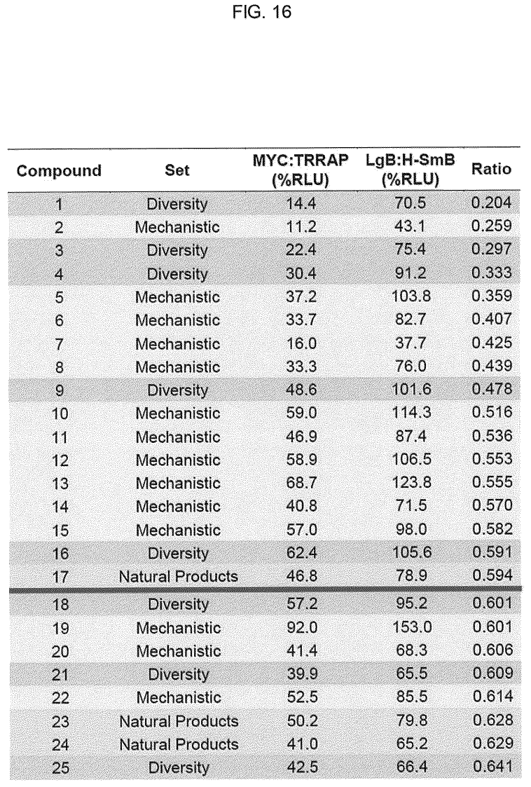

Afterwards, only molecules that reduced luminescence levels to <50% RLU were considered. This set of 46 compounds were incubated at 10 μM with vector-expressing cells. Luminescence complementation was measured and repeated in triplicate measurements. In addition, HeLa cells expressing LgB and SmB alone were incubated with the same compounds to rule out potentially artificial results due to inhibition of luciferase or its complementation. Molecules that induces any significant reduction in luminescence (<0.6) during this control assay were not considered further. Of 2947 molecules, 17 were chosen for further testing and ordered from the NCI/DTP Open Chemical Repository. FIG. 16 describes these chosen compounds.

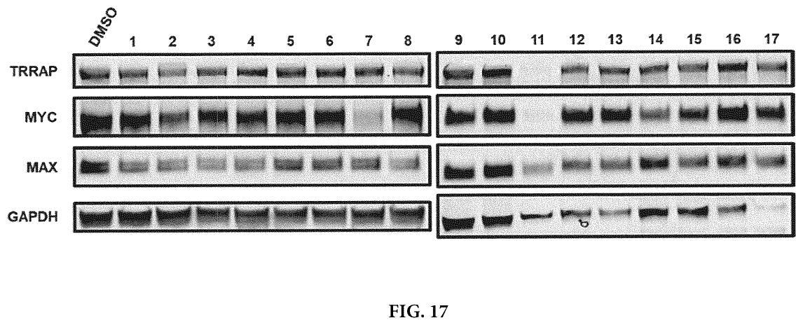

HeLa cells were subjected to the effects of incubation with each of these 17 compounds for 2 h. FIG. 17 presents a western blot of the effects of these compounds on the endogenous MYC, TRRAP, MAX, and GAPDH proteins. MAX protein levels were unaffected. All compounds except for 7 and 11 had no effect on the levels of MYC or TRRAP, suggesting that their effects are likely due to the inhibition of the MYC:TRRAP complex. However, the MYC- or MYC:TRRAP-specific effects observed by the presence of compound 7 or 11 can provide interesting insights into mechanisms involved in regulating MYC and TRRAP protein levels.

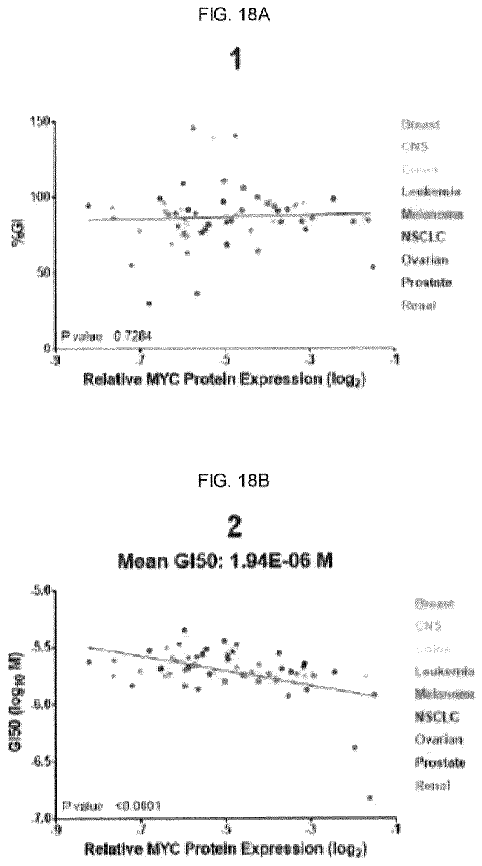

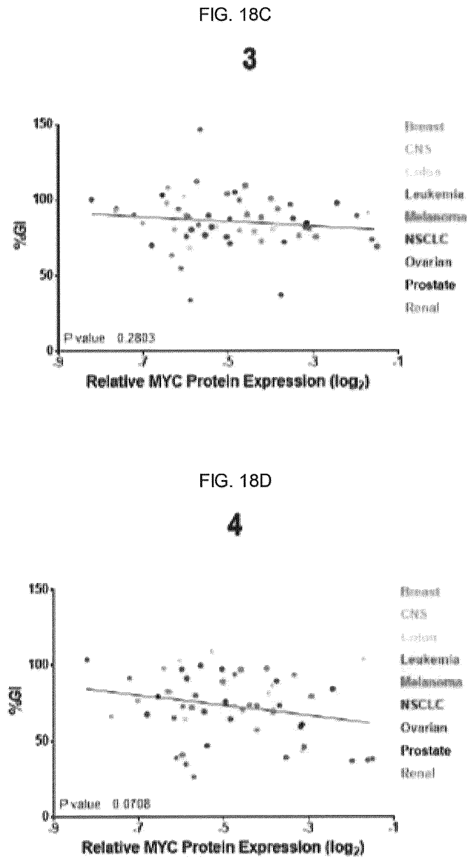

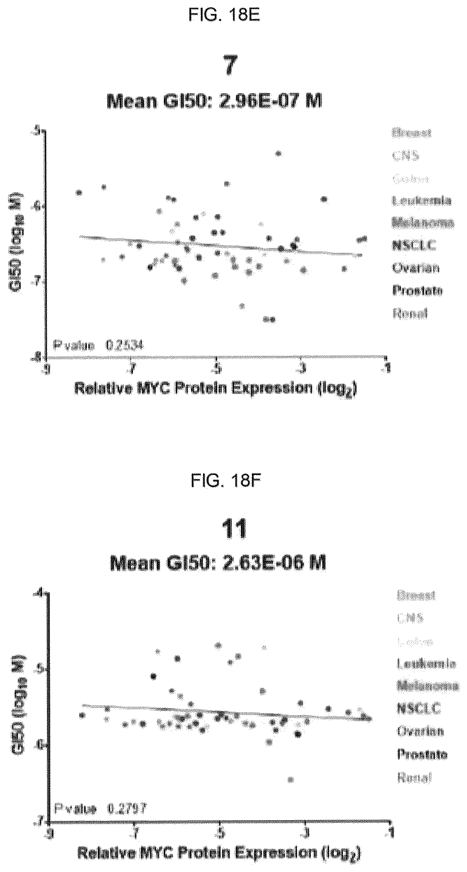

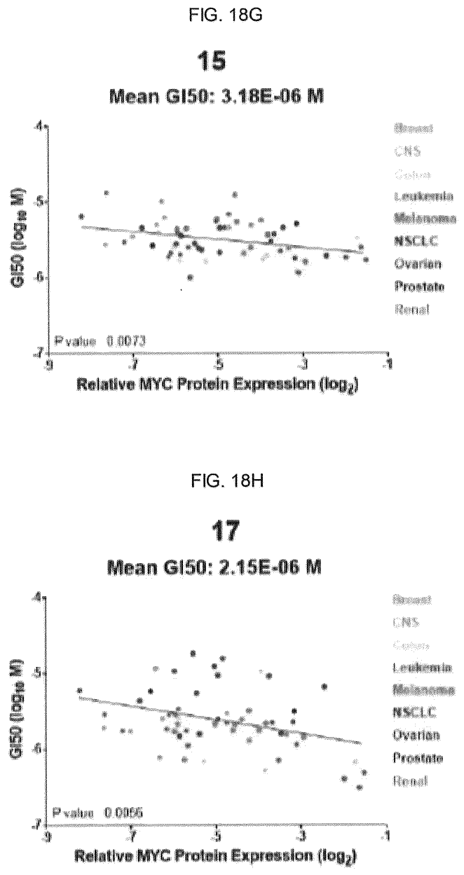

The NCI reports and freely shares G150 values for each of these compounds incubated with the NC160 panel of cell lines. They also report MYC protein expression data for the same panel of cell lines. A possible correlation between MYC expression and G150 values can exist that can help predict sensitivity of a cell line to each compound. Cell lines that need high levels of MYC might be more sensitive to a MYC:TRRAP inhibitor. FIG. 18A-FIG. 18H present some of the compounds from FIG. 16 that show a significant correlation between G150 and MYC protein expression and others that do not.

Compounds 1, 3, and 4 are structurally related but show very different G150 range and level of correlation with MYC expression (FIG. 18A, FIG. 18C, and FIG. 18D, respectively). Compounds 2, 15, and 17 show a significant correlation between their G150 and MYC expression (FIG. 18B, FIG. 18G, and FIG. 18H, respectively). However, these compounds are not structurally similar, suggesting that each could be affecting the MYC:TRRAP interaction in different manners. There could be some common geometrical motifs that are present in these compounds and a more thorough evaluation of their mechanism of action is warranted.

### Example 8: Co-IP Assay of Endogenous MYC:TRRAP Complex in the Presence of Inhibitors of a Binding Interaction Between MYC and TRRAP

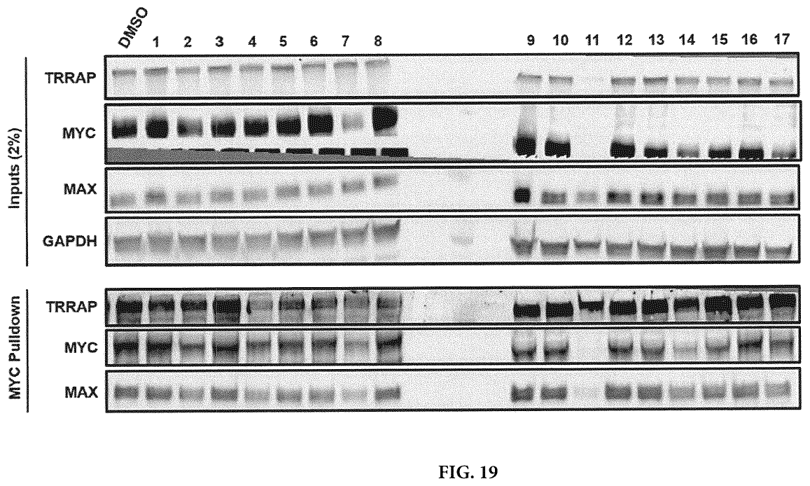

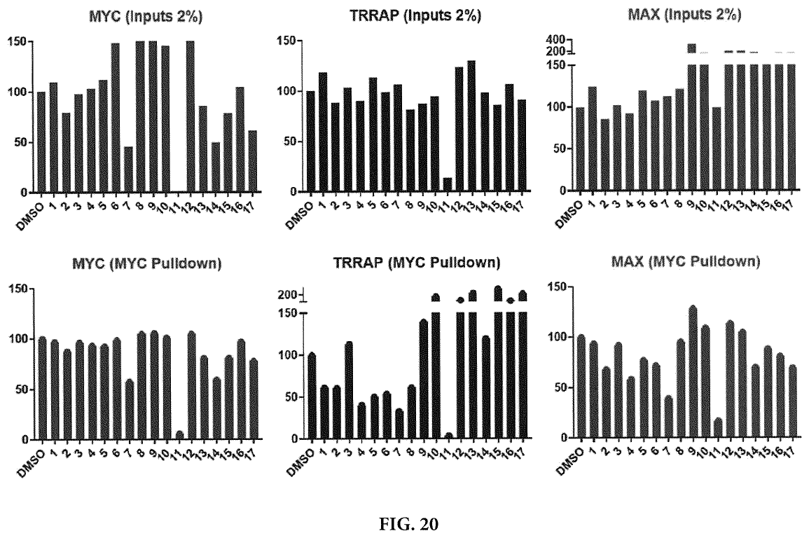

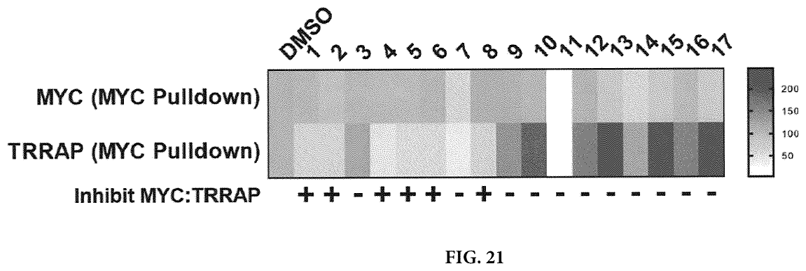

Co-IP experiments of the endogenous MYC:TRRAP complex were carried out to validate the results from the MYC:TRRAP in-cell luminescence complementation screen performed. HeLa cells were again subjected to the effects of incubation with each of the 17 compounds from FIG. 16 for 2 h before analysis. FIG. 19, FIG. 20, and FIG. 21 present the effects of these compounds on the endogenous MYC:TRRAP and MYC:MAX complexes. Like before, all compounds except for 7 and 11 had no effect on the levels of MYC or TRRAP. Incubation with compounds 1, 2, 4-6, and 8 showed a specific decrease in MYC:TRRAP co-IP and not MYC:MAX, while incubation with compounds 3, 9, 10, and 12-17 did not show any decrease in MYC:TRRAP co-IP. Incubation with some of the compounds that scored positive by the in-cell luminescence complementation screen had the predicted effect on the endogenous MYC:TRRAP complex. This validates the preliminary compound screen and presents the completion of a major milestone in the effort to therapeutically target MYC in cancer.

### Example 9: Determination of Inhibitory Concentration Curves and IC50s for Inhibitors of a Binding Interaction Between MYC and TRRAP

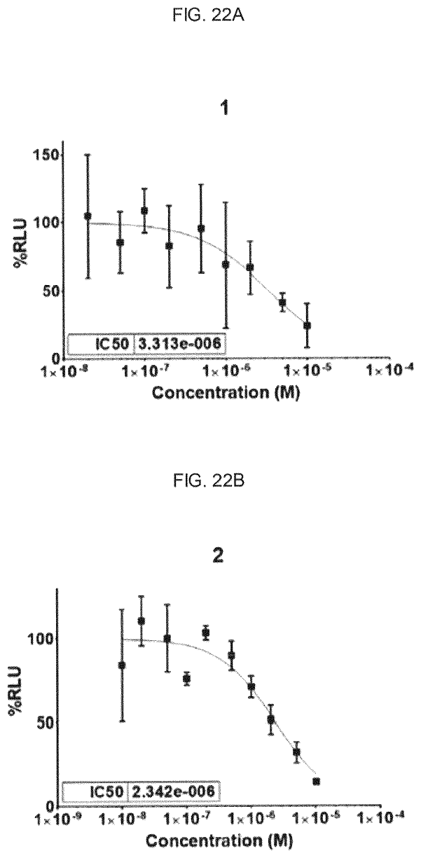

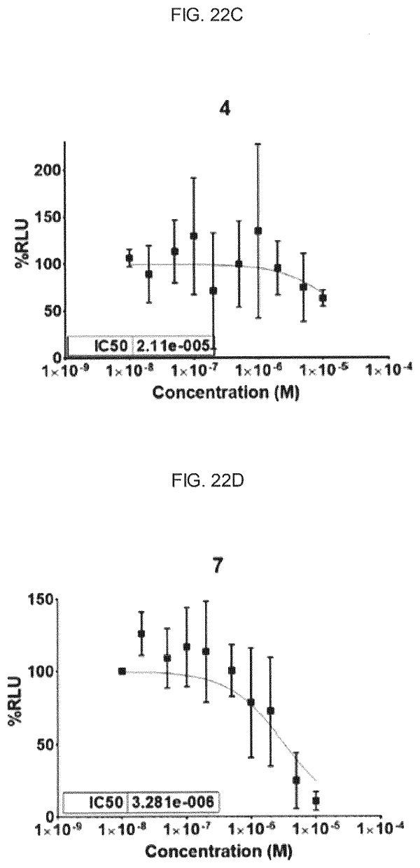

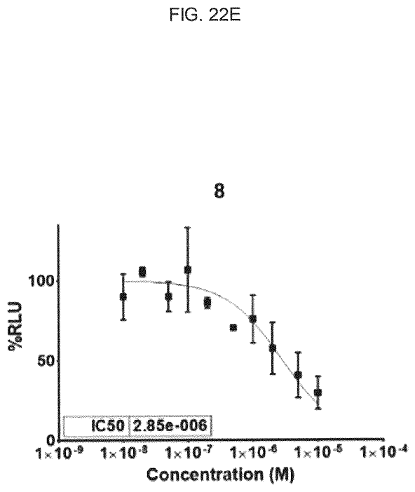

MYC:TRRAP in-cell luminescence complementation inhibition measurements were taken at varying concentrations of compounds 1, 2, 4, 7, and 8 to establish inhibitory concentration curves and IC50s for each compound (FIG. 22). Incubation with all compounds showed similar inhibition to the original large-scale screen, validating these results. Interestingly, compound 2 has a similar mean G150 for the NCI60 panel of cell lines and IC50 for MYC:TRRAP in-cell luminescence complementation inhibition. This suggests that inhibition of the endogenous MYC:TRRAP complex might be the mechanism of action for this compound's effects on growth inhibition on those cell lines. Although more experiments are required to further describe the mechanism of action for all these compounds, these results present convincing evidence that novel MYC inhibitor for treating cancer may be obtained using the disclosed assays.

### Example 10: Further and Screening of Other Inhibitory Compounds and “Derivatives” of Lead Compound

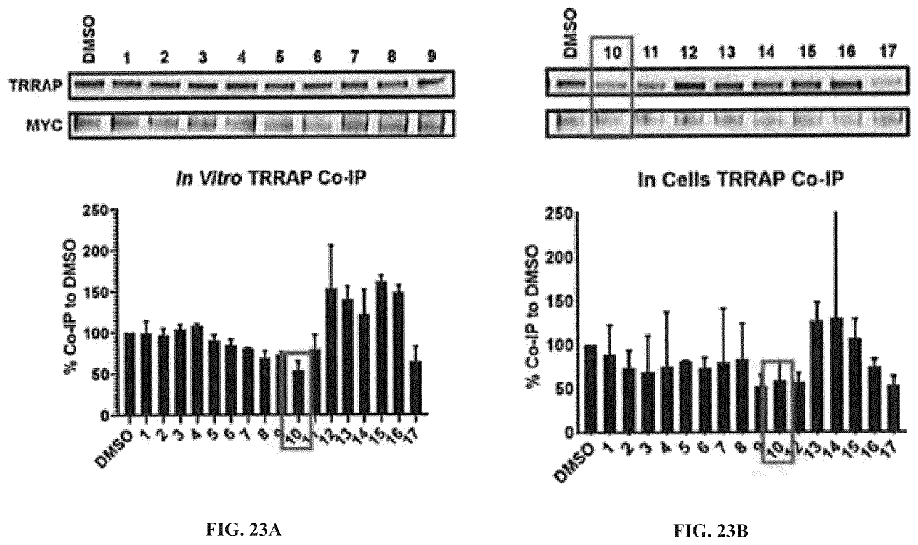

Using the luminescence assay above as a readout for the MYC:TRRAP interaction, we used a robotic liquid handler to evaluate 2987 additional compounds (25 μM). All primary hits were counter-screened for any activity against the luciferase enzyme itself and for any effects on the expression of the fusion proteins. We set a threshold of 50% inhibition to consider compounds further. Only 17 out of 2987 passed all these criteria (0.6%). Of these, four compounds dissociate TRRAP from MYC in vitro and inhibit MYC:TRRAP co-IP in cells (FIG. 23, lower). None of the compounds tested were overtly toxic at the concentrations tested in the brief (2 hr) treatment used for this experiment which is evident from the stable MYC expression.

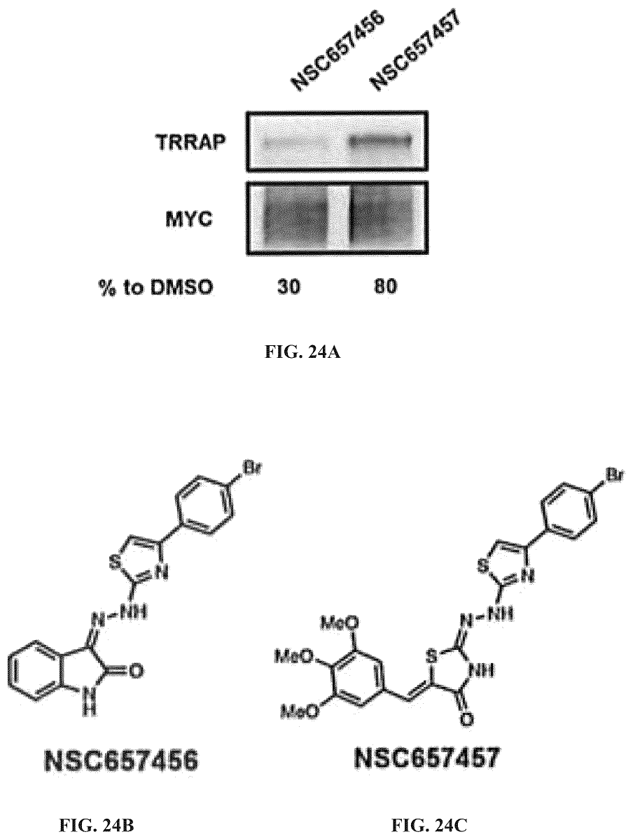

When these compounds were further characterized compound 10 (NSC657456) (FIG. 24, bottom left) gave the most consistent inhibition of MYC:TRRAP binding in multiple assays (~50%). Based thereon we concluded this compound is a good candidate for further chemical modification. By contrast, when we characterized other compounds like NSC657457, although they are very structurally similar to NSC657456, we observed that they do not inhibit the endogenous MYC:TRRAP interaction (FIG. 24).

We next screened a set of compounds which are closely structurally related to compound 10 (NSC657456). Our hope was that this subset of derivative compounds would identify more potent inhibitors of MYC:TRRAP complexes. Alternatively, our thinking was that chemical modifications that result in the disruption of the inhibitory capacity of NSC657456 would also provide useful information as this could shed further light into the most important chemical functional groups that are necessary for the inhibition of the MYC:TRRAP interaction.

Particularly, we assembled a similarity-based small molecule set composed of 40 compounds with >80% similarity to compound 10 (NSC657456). This was accomplished by searching the NCI's DTP Open Compound collection of about 250,000 compounds and their substructures using NCBI PubChem. We obtained these “derivative” compounds from the NCI and assayed them at three different concentrations using the in-cell luminescence complementation assay to identify any refined molecules that have higher affinity and specificity.

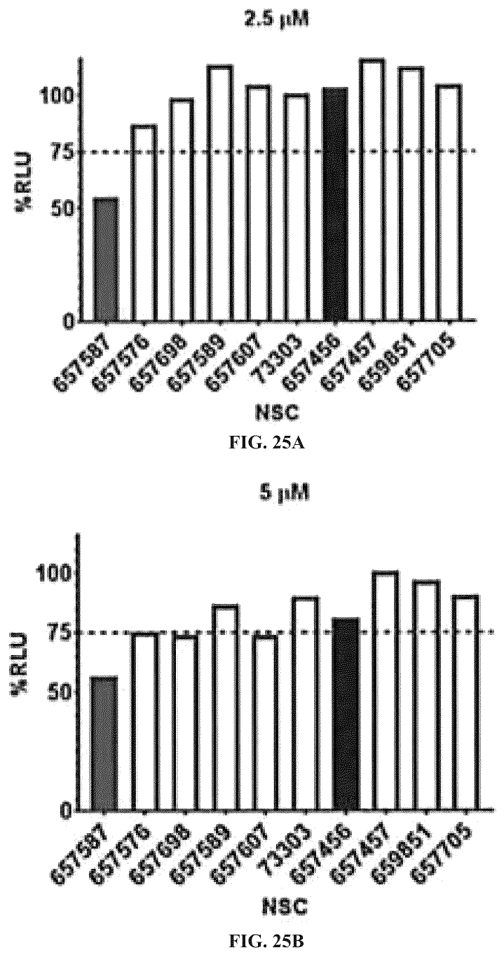

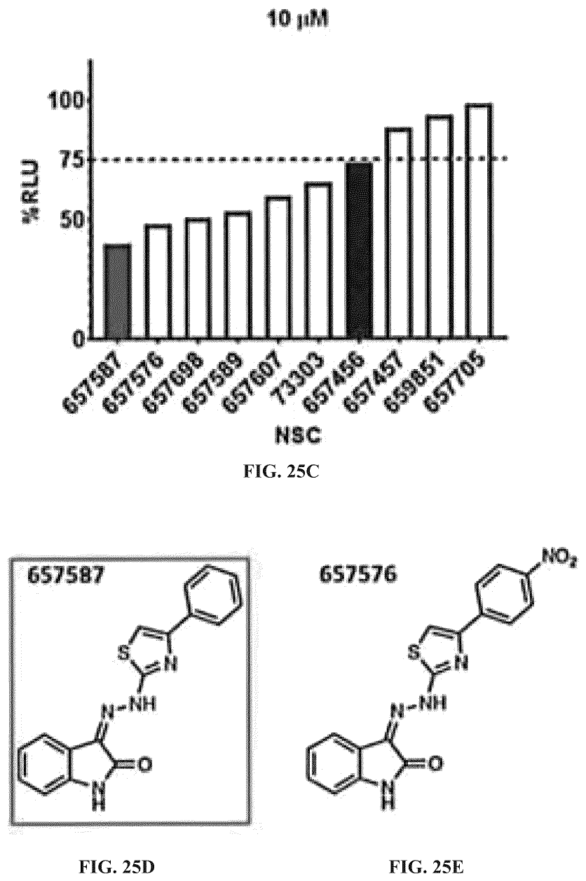

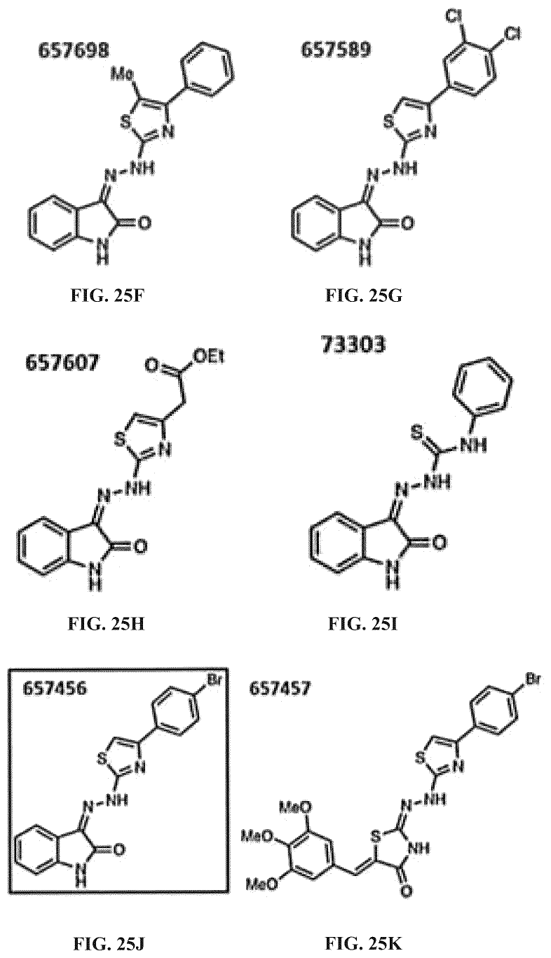

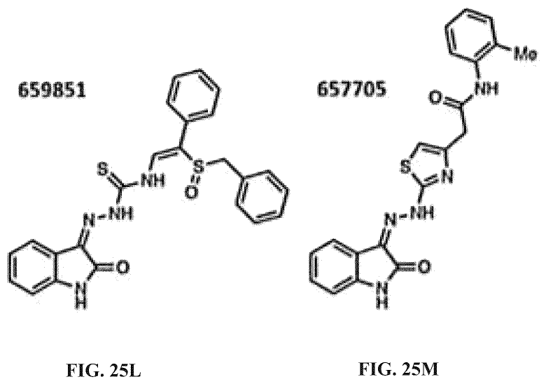

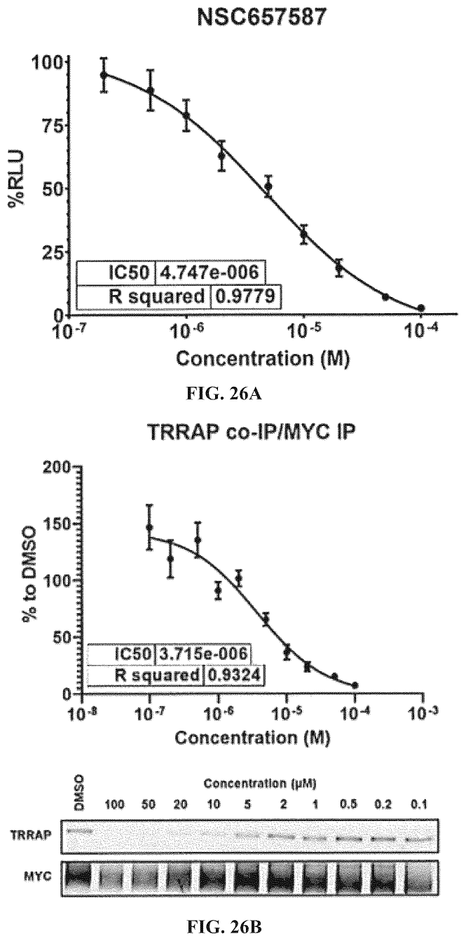

This assay identified compound NSC657587, which inhibits MYC:TRRAP complex formation at a lower concentration than NSC657456 (3-5 μM; FIG. 25, 26). These two compounds differ by a single bromine (Br) group on the benzene ring (FIG. 25). These data suggest that it is possible to increase the affinity for an already established inhibitor of the MYC:TRRAP interaction using small-molecule similarity screening. Thus, we have identified a compound that acts at a concentration similar to the best MYC:MAX inhibitors in only two iterations.

As we had hoped these results further helped us determine preliminary structure-activity relationships (SAR) and the most critical chemical groups involved in the inhibition of MYC:TRRAP. This information can be exploited in the rational design of new inhibitors with a much higher affinity. In short, both NSC657456 and NSC657587 are hydrazones derived from isatin. These types of functional groups are commonly present in approved drugs as well as experimental and investigational compounds. Modifications to the isatin structure result in extremely sensitive changes to the inhibitory capacity of these compounds to the MYC:TRRAP interaction (FIG. 24), suggesting it is the most important region. In addition, variants from these compounds are easily accessible with simple condensation chemistry. The introduction of further modifications outside of the core functional groups can increase the affinity of our exemplary lead compounds.

In particular other derivatives of compound 10 (NSC 657456) and compound 1 (NSC 657587) in Table 1 and Table 2, and in particular compounds which possess the 4 core structures set forth in Table 4 and Table 5 below, should result in the identification of other novel MYC inhibitors which may be used in cancer therapies.

TABLE 4
Derivatives of compound 10 (NSC 657456)
and our top lead compound 1024 (NSC
657587) that were tested and can
conceivably be tested based on our data
are divided in the following 4 general
skeleton subsets:

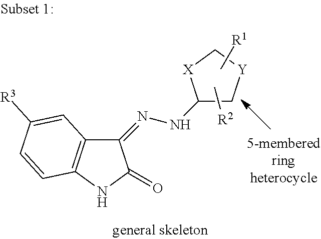

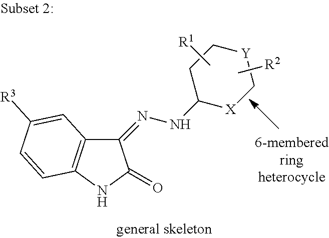

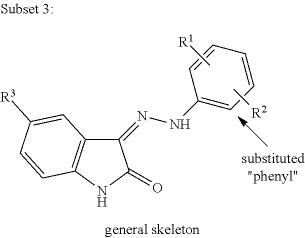

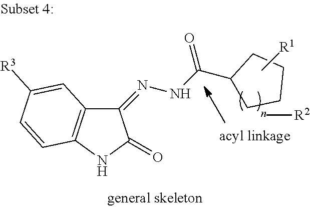

In the above structures the “R” substituents, i.e., R1, R2 and R3, optionally may be independently selected from at each occurrence, a bond, H, a substituted or unsubstituted: alkyl, alkenyl, alkynyl, phenyl, hydroxyl, carbonyl, aldehyde, haloformyl, carbonate ester, carboxylate, carboxyl, carboalkoxy, methoxy, hydroperoxyl, peroxy, ether, hemiacetal, hemiketal, acetal, orthoester, methylenedioxy, orthocarbonate ester, carboxylic anhydride, piperidine, pyridine, pyrrolidine, thiazole, imidazole, indole, tetrazole, carboxamide, primary amine, secondary amine, tertiary amine, quaternary amine, primary ketimine, secondary ketimine, primary aldimine, secondary aldimine, imide, azide, azo, cyanate, isocyanate, nitrate, nitrile, isonitrile, nitrosooxy, nitro, nitroso, oxime, pyridyl, carbamate, sulfhydryl, sulfide, disulfide, sulfinyl, sulfonyl, sulfino, sulfo, thiocyanate, isothiocyanate, carbonothioyl, carbothioic S-acid, carbothioic 0-acid, thiolester, thionoester, carbodithioic acid, carbodithio, phospphino, phosphono, phosphate, halo, fluoro, chloro, bromo, iodo, or any drug-like moiety or fragment.

Specific derivatives which possess one of the 4 core structures set forth above, which should result in the identification of other novel MYC inhibitors are contained in TABLE 5 below:

TABLE 5
EXEMPLARY DERIVATIVES OF LEAD COMPOUNDS

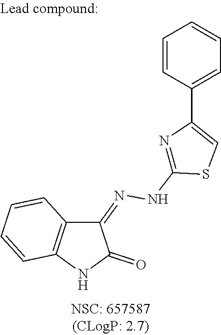 [SMILES: O=C1Nc2ccccc2/C1=N/Nc1nc(-c2ccccc2)cs1]

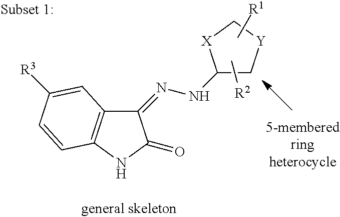

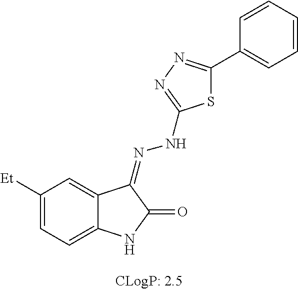 [SMILES: CCc1ccc2c(c1)/C(=N/Nc1nnc(-c3ccccc3)s1)C(=O)N2]

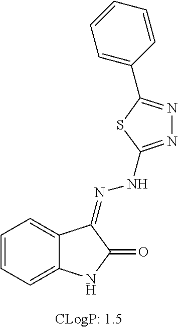 [SMILES: O=C1Nc2ccccc2/C1=N/Nc1nnc(-c2ccccc2)s1]

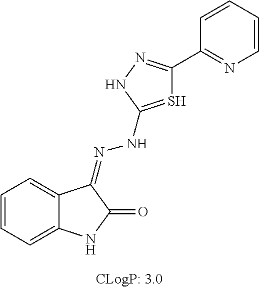

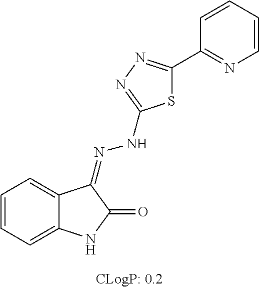 [SMILES: O=C1Nc2ccccc2/C1=N/Nc1nnc(-c2ccccn2)s1]

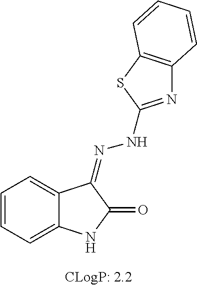 [SMILES: O=C1Nc2ccccc2/C1=N/Nc1nc2ccccc2s1]

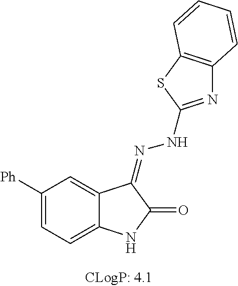 [SMILES: O=C1Nc2ccc(-c3ccccc3)cc2/C1=N/Nc1nc2ccccc2s1]

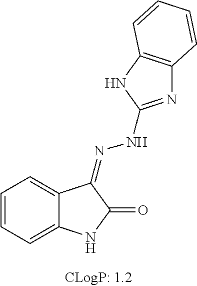 [SMILES: O=C1Nc2ccccc2/C1=N/Nc1nc2ccccc2[nH]1]

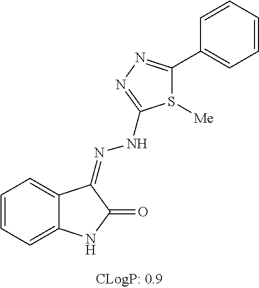

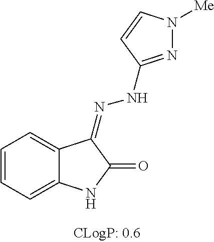 [SMILES: Cn1ccc(N/N=C2\C(=O)Nc3ccccc32)n1]

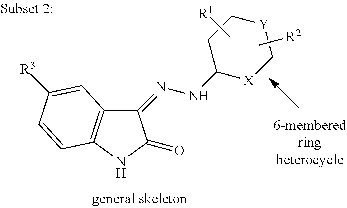

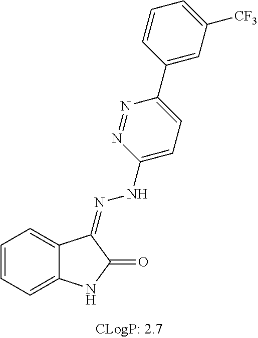 [SMILES: O=C1Nc2ccccc2/C1=N/Nc1ccc(-c2cccc(C(F)(F)F)c2)nn1]

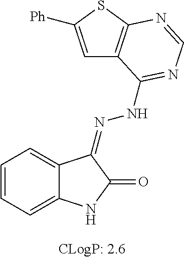 [SMILES: O=C1Nc2ccccc2/C1=N/Nc1ncnc2sc(-c3ccccc3)cc12]

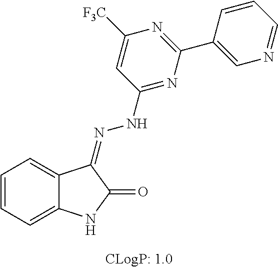 [SMILES: O=C1Nc2ccccc2/C1=N/Nc1cc(C(F)(F)F)nc(-c2cccnc2)n1]

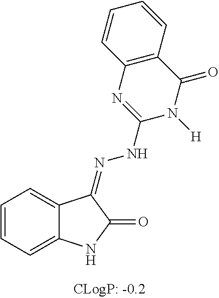 [SMILES: O=C1Nc2ccccc2/C1=N/Nc1nc2ccccc2c(=O)[nH]1]

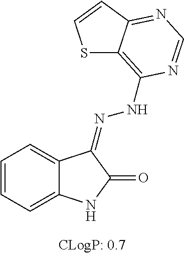 [SMILES: O=C1Nc2ccccc2/C1=N/Nc1ncnc2ccsc12]

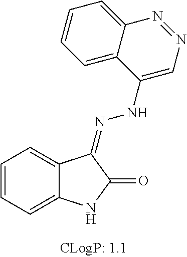 [SMILES: O=C1Nc2ccccc2/C1=N/Nc1cnnc2ccccc12]

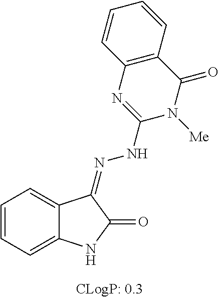 [SMILES: Cn1c(N/N=C2\C(=O)Nc3ccccc32)nc2ccccc2c1=O]

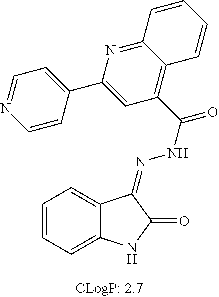 [SMILES: O=C1Nc2ccccc2/C1=N/NC(=O)c1cc(-c2ccncc2)nc2ccccc12]

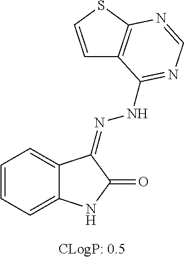 [SMILES: O=C1Nc2ccccc2/C1=N/Nc1ncnc2sccc12]

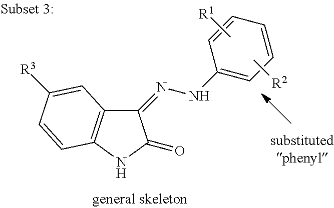

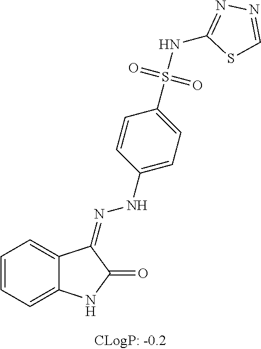 [SMILES: O=C1Nc2ccccc2/C1=N/Nc1ccc(S(=O)(=O)Nc2nncs2)cc1]

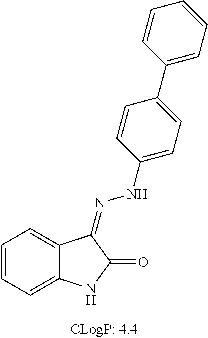 [SMILES: O=C1Nc2ccccc2/C1=N/Nc1ccc(-c2ccccc2)cc1]

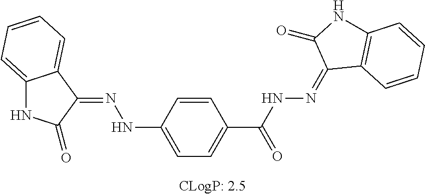 [SMILES: O=C1Nc2ccccc2/C1=N/NC(=O)c1ccc(N/N=C2\C(=O)Nc3ccccc32)cc1]

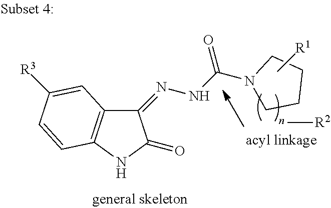

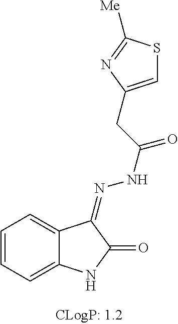 [SMILES: Cc1nc(CC(=O)N/N=C2\C(=O)Nc3ccccc32)cs1]

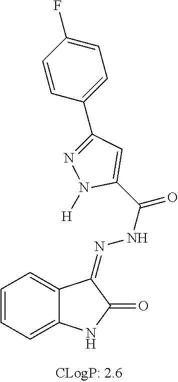 [SMILES: O=C1Nc2ccccc2/C1=N/NC(=O)c1cc(-c2ccc(F)cc2)n[nH]1]

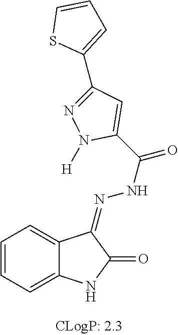 [SMILES: O=C1Nc2ccccc2/C1=N/NC(=O)c1cc(-c2cccs2)n[nH]1]

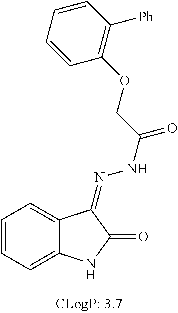 [SMILES: O=C(COc1ccccc1-c1ccccc1)N/N=C1\C(=O)Nc2ccccc21]

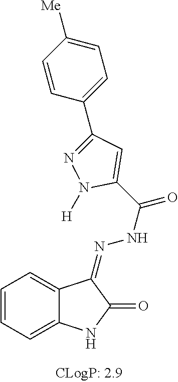 [SMILES: Cc1ccc(-c2cc(C(=O)N/N=C3\C(=O)Nc4ccccc43)[nH]n2)cc1]

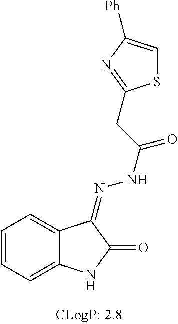 [SMILES: O=C(Cc1nc(-c2ccccc2)cs1)N/N=C1\C(=O)Nc2ccccc21]

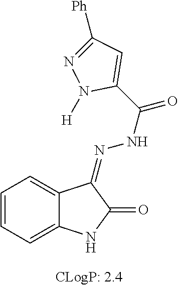 [SMILES: O=C1Nc2ccccc2/C1=N/NC(=O)c1cc(-c2ccccc2)n[nH]1]

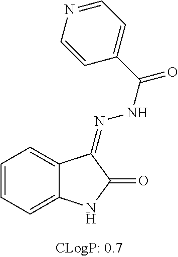 [SMILES: O=C1Nc2ccccc2/C1=N/NC(=O)c1ccncc1]

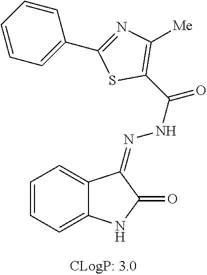 [SMILES: Cc1nc(-c2ccccc2)sc1C(=O)N/N=C1\C(=O)Nc2ccccc21]

### Example 11: Improved Transfection Protocol for Identifying Inhibitory Compounds

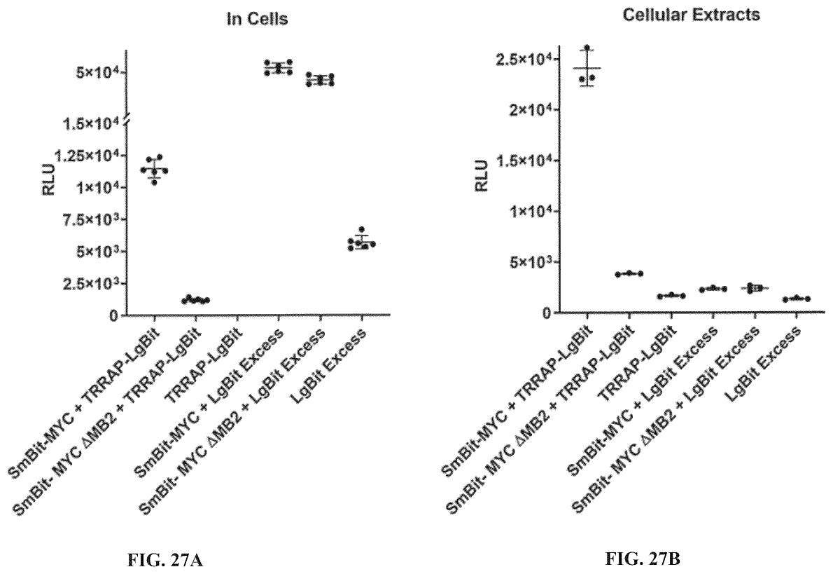

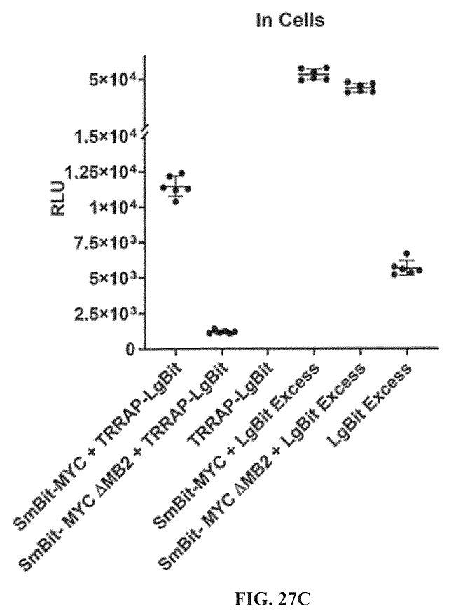

Using Expi293 cells from ThermoFisher, a new transfection protocol was developed (shown schematically in FIG. 27) that elicits 100-fold more luminescent signal while maintaining the same signal to noise ratio. Particularly, as shown in FIG. 27 the use of a suspension of 293 cells (which cell suspensions are cultured using a CO₂ shaker incubator) provides for high transfection efficiency and further advantageously these cells can grow up to 6000 cells/uL. By way of comparison, when we used HeLa cells we typically plated about 5000 cells per well (in 1536 plates) in 8 uL; by contrast, using Expi 293 cells we were able to plate 20,000 cells per well in 4 uL.

This will provide a much higher signal and reduce the amount of NanoGlo® needed by at least half, resulting in a lower cost per plate. Also, unlike HeLa cells, Expi 293 cells do not need to be lifted or attached to a substrate before adding the compounds used for screening. Accordingly, we are able to transfect cells in large liter batches and plate the cells into wells already containing compounds, making it possible to obtain all measurements in a single day of automation instead of two.

Additionally, the use of Expi 293 cell suspensions provides for reduced integration times. Particularly for the measurements shown in FIG. 27 a 2s integration time was used for HeLa cells (2s per measurement), while for the Expi 293 cells a 0.5s integration time was used. This was feasible because of the much higher signal using transfected Expi 293 cells which substantially reduces the required integration time per plate. Expi 293 cells can be used for both cell and in vitro measurements.

The following references and other references cited in the application are incorporated by reference in their entirety herein.

### REFERENCES

1. Weinstein I B and Joe A, Felsher D. “Oncogene Addiction”, 2008, Cancer Res. 68(9):3077-80.
2. Bradner J E and Hnisz D, Young R A., “Transcriptional Addiction in Cancer”, 2017, Cell February; 168(4):629-43.
3. Dang C V et al., “The c-Myc target gene network”, 2006, Semin Cancer Biol. 16(4):253-64.
4. Shachaf C M et al., “Genomic and proteomic analysis reveals a threshold level of MYC required for tumor maintenance”, Cancer Res. 2008 Jul. 1; 68(13):5132-42.
5. Yekkala K and Baudino T A, “Inhibition of intestinal polyposis with reduced angiogenesis in ApcMin/+ mice due to decreases in c-Myc expression”, 2007, Mol Cancer Res 5(12):1296-303.
6. Meyer N and Penn L Z, “Reflecting on 25 years with MYC”, 2008, Nat Rev. 8(12):976-90.
7. Charron J et al., “Embryonic lethality in mice homozygous for a targeted disruption of the N-myc gene”, 1992, Genes Dev.6(12A):2248-57.
8. Knoepfler P S et al., “N-myc is essential during neurogenesis for the rapid expansion of progenitor cell populations and the inhibition of neuronal differentiation”, 2002, Genes Dev. 16(20):2699-712.
9. Beroukhim R et al. “The landscape of somatic copy-number alteration across human cancers”, 2010, Nature. 463(7283):899-905.
10. Nair S K and Burley S K, “X-ray structures of Myc-Max 547 and Mad-Max recognizing DNA: Molecular bases of regulation by proto-oncogenic transcription factors”, 2003, Cell 112(2):193-205.
11. Brownlie P et al. “The crystal structure of an intact human Max-DNA complex: new insights into mechanisms of transcriptional control”, 1997, Structure 5(4):509-20.
12. Cole M D and Cowling V H, “Transcription-independent functions of MYC: regulation of translation and DNA replication”, 2008, Nat Rev Mol Cell Biol. 9(10):810-5.
13. Brown S J, Cole M D, Erives A J, “Evolution of the holozoan ribosome biogenesis regulon”, 2008, BMC Genomics 9:442.
14. Cowling V H et al., “A conserved Myc protein domain, MBIV, regulates DNA binding, apoptosis, transformation, and G2 arrest”, Mol Cell Biol. 2006 June; 26(11):4226-39.
15. McKeown M R and Bradner J E, “Therapeutic strategies to inhibit MYC”, 2014, Cold Spring Harb Perspect Med. 4(10).
16. Nikiforov M A et al., “TRRAP dependent and TRRAP-independent transcriptional activation by Myc family oncoproteins”, 2002, Mol Cell Biol. 22(14):5054-63.
17. Carabet L A et al., “Therapeutic Inhibition of Myc in Cancer. Structural Bases and Computer-Aided Drug Discovery Approaches”, 2018, Inti Mol Sci. 20(1).
18. Posternak V and Cole M D, “Strategically targeting MYC in cancer”, F1000 Research. 2016; 5.
19. Whitfield J R et al., “Strategies to Inhibit Myc and Their Clinical Applicability”, 2017, Front Cell Dev Biol. 5:10.
20. Brough D E et al., “An essential domain of the c-myc protein interacts with a nuclear factor that is also required for E1A-mediated transformation”, 1995 Mol Cell Biol. 15(3):1536-44.
21. Dang C V. “MYC on the path to cancer”, 2012 Cell 149(1):22-35.
22. Cowling V H, Cole M D, “Mechanism of transcriptional 573 activation by the Myc oncoproteins”, 2006, Semin Cancer Biol. 16(4):242-52.
23. McMahon S B et al., “The novel ATM576 related protein TRRAP is an essential cofactor for the c-Myc and E2F oncoproteins”, 1998 Cell 94(3):363-74.
24. Kalkat M et al., “MYC Protein Interactome Profiling Reveals Functionally Distinct Regions that Cooperate to Drive Tumorigenesis”, 2018, Mol Cell. 72(5):836-848.e7.
25. Murr R et al., “Orchestration of chromatin-based processes: mind the TRRAP”, 2007, Oncogene 26(37):5358-72.
26. Baretic D, Williams R L, “PIKKs—the solenoid nest where partners and kinases meet” 2014, Curr Opin Struct Biol.29:134-42.
27. Lempisinen H, Halazonetis T D, “Emerging common themes in regulation of PIKKs and PI3Ks”, 2009, EMBOJ. 28(20):3067-73.
28. Saleh A et al., “Tra1p is a component of the yeast Ada.Spt transcriptional regulatory complexes”, 1998, J Biol Chem. 273(41):26559-65.
29. Doyon Y, Côté J, “The highly conserved and multifunctional NuA4 HAT complex”, 2004, Curr Opin Genet Dev. 14(2):147-54.
30. Grant P A, Schieltz D, Pray-Grant M G, Yates J R, Workman J L. The ATM-related cofactor Tra1 is a component of the purified SAGA complex. Mol Cell. 1998 December; 2(6):863-7.
31. Herceg Z et al., “Disruption of Trrap causes early embryonic lethality and defects in cell cycle progression”, 2001 Nat Genet. 29(2):206-212
32. Murugan et al., “Mutational analysis of the GNA11, MMP27, FGD1, TRRAP and GRM3 genes in thyroid cancer”, 2013, Oncol Lett. 6(2):437-41.
33. Wei X et al., “599 Exome sequencing identifies GRIN2A as frequently mutated in melanoma”, 2011, Nat Genet. 43(5):442-6.
34. McMahon S B et al., “The essential cofactor TRRAP recruits the histone acetyltransferase hGCN5 to c-Myc”, 2000, Mol Cell Biol. 20(2):556-62.
35. Brown C E et al., “Recruitment of HAT complexes by direct activator interactions with the ATM-related Tra1 subunit”, 2001, Science. 292(5525):2333-7.
36. Ard P G et al., “Transcriptional regulation of the mdm2 oncogene by p53 requires TRRAP acetyltransferase complexes”, 2002, Mol Cell Biol. 22(16):5650-61.
37. Lang S E, Hearing P, “The adenovirus E1A oncoprotein recruits the cellular TRRAP/GCN5 histone acetyltransferase complex”, 2003, Oncogene 22(18):2836-41.
38. Das C, Tyler J K, “Histone exchange and histone modifications during transcription and aging”, 2013, Biochim Biophys Acta.1819(3-4):332-42.
39. Park J, et al., “The ATM-related domain of TRRAP is required for histone acetyltransferase recruitment and Myc-dependent oncogenesis”, 2001, Genes Dev. 15(13):1619-24.
40. Diaz-Santin L M et al., “Cryo-EM structure of the SAGA and NuA4 coactivator subunit Tra1 at 3.7 angstrom resolution” 2017, eLife 02:6.
41. Knutson B A, Hahn S, “Domains of Tra1 important for activator recruitment and transcription coactivator functions of SAGA and NuA4 complexes”, 2011, Mol Cell Biol. 31(4):818-31.
42. Lee W et al., “Integrative NMR for biomolecular research”, 2016, J Biomol NMR. 64(4):307-32.
43. McEwan I J et al., “Functional interaction of the c-Myc transactivation domain with the TATA binding protein: evidence for an induced fit model of transactivation domain folding”, 1996, Biochemistry, 35(29):9584-93.
44. Tu W B et al., “Myc and its interactors take shape” 2015 Biochim Biophys Acta BBA—Gene Regul Mech. 1849(5):469-83.
45. Krois A S et al., “Recognition of the disordered p53 transactivation domain by the transcriptional adapter zinc finger domains of CREB-binding protein”, 2016, Proc Natl Acad Sci. 113(13):E1853-62.
46. Chen X, et al., “Fusion protein linkers: property, design and functionality”, 2013, Adv Drug Deliv Rev 65(10):1357-69.
47. Lee W et al., “PONDEROSA, an automated 3D-NOESY peak picking program, enables automated protein structure determination”, 2011, Bioinformatics 27(12):1727-8.
48. Neidigh J W, et al., “Exendin-4 and glucagon-like peptide-1: NMR structural comparisons in the solution and micelle-associated states”, 2001, Biochemistry 40(44):13188-200.
49. Upadhyay V et al., “Recovery of bioactive protein from bacterial inclusion bodies using trifluoroethanol as solubilization agent”, 2016, Microb Cell Factories 15:100.
50. Sonnichsen F D et al., “Effect of trifluoroethanol on protein secondary structure: an NMR and C D study using a synthetic actin peptide”, 1992, Biochemistry 31(37):8790-8.
51. Gast K et al., “Trifluoroethanol-induced conformational transitions of proteins: Insights gained from the differences between α-lactalbumin and ribonuclease A”, 2008, Protein Sci. 8(3):625-34.
52. Felsher D W, “MYC Inactivation Elicits Oncogene Addiction through Both Tumor Cell-Intrinsic and Host-Dependent Mechanisms”, 2010, Genes Cancer 1(6):597-604.
53. Fiorentino F P et al., “Growth suppression by MYC inhibition in small cell lung cancer cells with TP53 and RB1 inactivation”, 2016, Oncotarget 7(21):31014-28.
54. Soucek L et al., “Inhibition of Myc family proteins eradicates KRas-driven lung cancer in mice”. 2013, Genes Dev. 27(5):504-13.
55. Wang X et al., “Architecture of the Saccharomyces cerevisiae NuA4/TIP60 complex”, 2018, Nat Commun 9(1):1147.
56. Rivera-Calzada A, et al., “Structure and Assembly of the PI3K-like Protein Kinases (PIKKs) Revealed by Electron Microscopy”, 2015, AIMS Biophys 2(2):36-57.
57. Sibanda B L et al., “DNA-PKcs structure suggests an allosteric mechanism modulating DNA double-strand break repair”, 2017 Science 355(6324):520-4.
58. Daub H et al., “Kinase-selective enrichment enables quantitative phosphoproteomics of the kinome across the cell cycle”, 2008, Mol Cell 31(3):438-48.
59. Dephoure N et al., “A quantitative atlas of mitotic phosphorylation”, 2008, Proc Natl Acad Sci USA. 105(31):10762-7.
60. Zhou H et al., “Toward a comprehensive characterization of a human cancer cell phosphoproteome”, 2013, J Proteome Res 12(1):260-71.
61. Allard S et al., “NuA4, an essential transcription adaptor/histone H4 acetyltransferase complex containing Esa1p and the ATM-related cofactor Tra1p”, 1999, EMBOJ 18(18):5108-19.
62. Grant P A et al., “Yeast Gcn5 functions in two multisubunit complexes to acetylate nucleosomal histones: characterization of an Ada complex and the SAGA (Spt/Ada) complex”, 1997, Genes Dev. 11(13):1640-50.
63. Côté J et al., “Basic analysis of transcription factor binding to nucleosomes”, 1995, In: Methods in Molecular Genetics Elsevier; pp. 108-28.
64. Drozdetskiy A et al., “Pred4: a protein secondary structure prediction server”, 2015, Nucleic Acids Res 43(W1):W389-94.
65. Hall, M. P et al., “Engineered luciferase reporter from a deep sea shrimp utilizing a novel imidazopyrazinone substrate”, 2012, ACS Chem. Biol. 7, 1848-1857.
66. Cerami J et al. “The cBio Cancer Genomics Portal: An Open Platform for Exploring Multidimensional Cancer Genomics Data: FIG. 1”, 2012, Cancer Discov. 2, 401-404.
67. Gao, J et al. “Integrative analysis of complex cancer genomics and clinical profiles using the cBioPortal”, 2013, Sci. Signal. 6:11.

1. A method for identifying a chemical compound which inhibits the binding interaction between MYC transcription factor and Transformation/Transcription Domain-Associated Protein (TRRAP), comprising:
a) forming a MYC:TRRAP complex having a MYC:TRRAP binding interaction;
b) directly and/or indirectly detecting the MYC:TRRAP complex and/or the MYC:TRRAP binding interaction to determine a baseline measurement for the MYC:TRRAP complex and/or the MYC:TRRAP binding interaction;
c) introducing a compound prior to or after forming a MYC:TRRAP complex having a MYC:TRRAP binding interaction; and
d) determining an absence or a reduction of the MYC:TRRAP complex and/or the MYC:TRRAP binding interaction after the chemical compound has been introduced compared to the baseline measurement, wherein the absence or the reduction of the MYC:TRRAP complex and/or the MYC:TRRAP binding interaction indicates that the chemical compound is an inhibitor of the binding interaction between MYC and TRRAP.

2. The method of claim 1, comprising one or more of the following:
(i) MYC comprises an amino acid sequence which is at least 75% identical, at least 80% identical, at least 85% identical, at least 90% identical, at least 95% identical, at least 98% identical, or at least 99% identical to SEQ ID NO: 2 or to another mammalian MYC amino acid sequence;
(ii) TRRAP comprises an amino acid sequence which is at least 75% identical, at least 80% identical, at least 85% identical, at least 90% identical, at least 95% identical, at least 98% identical, or at least 99% identical to SEQ ID NO: 4 or to another mammalian TRRAP amino acid sequence;
(iii) the MYC:TRRAP complex is formed in an in vitro environment, a cell, or in a non-human animal selected from C. elegans, D. melanogaster, a zebrafish, a rodent, and a non-human primate;
(iv) the cell in (iii) is selected from a human cell, a mammalian cell, an insect cell, a yeast cell, and a bacterial cell;
(v) the cell is a HeLa cell, a 293 cell, an Expi293 cell or a Expi293 cell suspension;
(vi) the compound is a small molecule, optionally comprising a hydrazone, urea, thiourea, ketone, sugar, lipid, amino acid, fatty acid, nucleotide, peptide, phenol, alcohol, polyketide, glycoside, alkaloid, phenazine, polyketide, terpene, tetrapyrrole;
(vii) the compound is one of the compounds in Table 1, Table 2 or Table 5 and/or one comprising one of the generic structures set forth in Table 4, optionally wherein the “R” substituents, i.e., R1, R2 and R3, are selected from at each occurrence, a bond, H, a substituted or unsubstituted: alkyl, alkenyl, alkynyl, phenyl, hydroxyl, carbonyl, aldehyde, haloformyl, carbonate ester, carboxylate, carboxyl, carboalkoxy, methoxy, hydroperoxyl, peroxy, ether, hemiacetal, hemiketal, acetal, orthoester, methylenedioxy, orthocarbonate ester, carboxylic anhydride, piperidine, pyridine, pyrrolidine, thiazole, imidazole, indole, tetrazole, carboxamide, primary amine, secondary amine, tertiary amine, quaternary amine, primary ketimine, secondary ketimine, primary aldimine, secondary aldimine, imide, azide, azo, cyanate, isocyanate, nitrate, nitrile, isonitrile, nitrosooxy, nitro, nitroso, oxime, pyridyl, carbamate, sulfhydryl, sulfide, disulfide, sulfinyl, sulfonyl, sulfino, sulfo, thiocyanate, isothiocyanate, carbonothioyl, carbothioic S-acid, carbothioic O-acid, thiolester, thionoester, carbodithioic acid, carbodithio, phospphino, phosphono, phosphate, halo, fluoro, chloro, bromo, iodo, or any drug-like moiety or fragment; or
(viii) or any combination of the foregoing.

3. The method of claim 1, wherein the MYC:TRRAP complex comprises:
a full-length MYC or a MYC fragment and a full-length TRRAP or a TRRAP fragment; or
a MYC-TRRAP fusion comprising:
the full-length MYC or the MYC fragment,
a linker, and
the full-length TRRAP or the TRRAP fragment;
wherein the MYC fragment comprises a minimal MYC region and the TRRAP fragment comprises a minimal TRRAP region, wherein:
the minimal MYC region is a MYC MB2 domain; and
the minimal TRRAP region is a TRRAP 2033-2088 region; and
wherein any one or more of the full-length MYC, the MYC fragment, the full-length TRRAP, and the TRRAP fragment optionally comprises:
an affinity tag,
a detectable label, and/or
a distinct protein, protein domain, or protein fragment useful for purification, identification, and/or complementation.

4. The method of claim 1, wherein
(i) the chemical compound is isolated or comprised in a mixture of chemical compounds;
(ii) the chemical compound comprises a small-molecule organic chemical compound; and/or
(iii) the chemical compound is selected from a small-molecule chemical compound library;
(iv) or any combination of the foregoing.

5. The method of claim 1, further comprising:
(i) determining the specificity of the chemical compound for inhibiting the binding interaction between MYC and TRRAP by testing the ability of the chemical compound to inhibit a binding interaction between MYC and the MYC-associated factor MAX in at least one in vitro or in vivo assay;
(ii) conducting a cell-based protein-fragment complementation assay to detect the MYC:TRRAP complex and/or the MYC:TRRAP binding interaction;
(iii) conducting a cell-based protein-fragment complementation assay to detect the MYC:TRRAP complex and/or the MYC:TRRAP binding interaction, wherein the cell-based protein-fragment complementation assay is a luminescence complementation assay;
(iv) conducting a cell-based protein-fragment complementation assay to detect the MYC:TRRAP complex and/or the MYC:TRRAP binding interaction, wherein the cell-based protein-fragment complementation assay is a luminescence complementation assay, wherein the luminescence complementation assay comprises:
an SmB-luciferase-MYC fusion comprising an N-terminal SmB-luciferase fragment and a C-terminal full-length MYC or a C-terminal MYC fragment; and
a TRRAP-LgB-luciferase fusion comprising an N-terminal TRRAP fragment and a C-terminal LgB-luciferase fragment;
wherein the SmB-luciferase-MYC fusion and the TRRAP-LgB-luciferase fusion form the MYC:TRRAP complex, whereby the SmB-luciferase fragment and the LgB-luciferase fragment form a functional luciferase enzyme which generates a luminescence signal in the presence of a luciferase substrate.

6. The method of claim 1, wherein
(i) the MYC fragment is a MYC 1-190 fragment and the TRRAP fragment is a TRRAP 2033-2283 fragment;
(ii) the functional luciferase enzyme is a 19.1 kDa luciferase enzyme derived from Oplophorus gracilirostrisl;
(iii) the SmB-luciferase-MYC fusion and the TRRAP-LgB-luciferase fusion are each expressed in the cell from a mammalian expression vector comprising a constitutive promoter, optionally a CMV promoter;
(iv) the expression level of the SmB-luciferase-MYC fusion and the expression level of the TRRAP-LgB-luciferase fusion are substantially equal;
(v) the cell comprises a HeLa cell;
(vi) the cell comprises an Epti293 cell or an Expi293 cell suspension;
(v) the luciferase substrate is furimazine;
(vi) the luminescence complementation assay further comprises detecting a false positive result caused by direct inhibition of the luciferase activity or by inhibition of the complementation of the SmB-luciferase and LgB-luciferase fragments;
(vii) the cell further expresses a fluorescence reporter, wherein the fluorescence reporter is used to normalize transfection efficiency and cell number, optionally where the fluorescence reporter is EGFP;
(viii) the chemical compound is introduced at different concentrations, optionally ranging from 10 nM to 100 μM;
(ix) the method comprises determining an IC50 value for the tested chemical compound or compounds;
(x) the chemical compound is introduced at a concentration reduces the luminescence signal by at least 50%;
(xi) the chemical compound is introduced at a concentration of 1-500 μM, 5-100 μM, 10-50 μM or 25 μM which optionally reduces the luminescence signal by at least 20, 30, 40 or 50%;
(xii) the assay uses a transfected suspension of 293 cells, optionally plated at about 10,000-20,000 cells per well;
(xiii) the assay uses transfected suspensions of Expi 293 cells, optionally cultured using a CO₂ shaker incubator, further optionally wherein about 20,000 cells per plate in about 4 uL volume are used, thereby reducing the amount of Nanoglo required for detection and/or decreasing the integration time by about 4-fold in comparison to transfected HeLa cells when these cells are used in high throughput screening methods (e.g., about 2s per measurement in comparison to 0.5s per measurement for Expi 293 cells); or
(xiv) any combination of (i) to (xiii).

7. The method of claim 1, wherein the chemical compound which has been identified as reducing the formation of the MYC:TRRAP complex and/or to block the MYC:TRRAP binding interaction is assessed for its antitumor efficacy in an in vitro or in vivo tumor model.

8. (canceled)

9. (canceled)

10. The method of claim 1 comprising one or more of the following:
(i) co-purification of the MYC:TRRAP complex from cells to detect the MYC:TRRAP complex and/or amount thereof and/or to detect the effect of the compound on the MYC:TRRAP binding interaction;
(ii) the cells are selected from human cells, mammalian cells, insect cells, yeast cells, and bacterial cells;
(iii) the cells are HeLa cells, 293 cells or Expi 293 cells;
(iv) co-immunoprecipitation of the MYC:TRRAP complex from a cell lysate to detect the MYC:TRRAP complex and/or the MYC:TRRAP binding interaction;
(v) the co-immunoprecipitated MYC:TRRAP complex comprises the full-length MYC or MYC fragment having a first affinity tag and the full-length TRRAP or TRRAP fragment having a second affinity tag and, wherein the full-length MYC or MYC fragment having a first affinity tag and the full-length TRRAP or TRRAP fragment having a second affinity tag are co-expressed in the cell; and the first affinity tag and the second affinity tag are different;
(vi) the co-immunoprecipitated MYC:TRRAP complex comprises the full-length MYC or MYC fragment having a first affinity tag wherein the full-length MYC or MYC fragment having a first affinity tag is expressed in the cell and co-immunoprecipitates endogenous TRRAP; or the full-length TRRAP or TRRAP fragment having a first affinity tag wherein, the full-length TRRAP or TRRAP fragment having a first affinity tag is expressed in the cell and co-immunoprecipitates endogenous MYC;
(vii) the first affinity tag and the second affinity tag in the co-immunoprecipitated MYC:TRRAP complex are selected from a PYO tag and a FLAG tag, wherein the first affinity tag and the second affinity tag are different;
(viii) the MYC:TRRAP complex is detected by Western Blot analysis using an anti-MYC antibody, an anti-TRRAP antibody, an anti-FLAG antibody, and/or an anti-PYO antibody;
(ix) the MYC fragment in the MYC:TRRAP complex is a MYC 1-190 fragment and the TRRAP fragment is a TRRAP 2033-2283 fragment;
(x) the cell lysate is optionally selected from a human cell lysate, a mammalian cell lysate, an insect cell lysate, a yeast cell lysate, and a bacterial cell lysate;
(xi) the cell lysate is a HeLa, HEK293T, 293 or Expi293 cell lysate;
(xii) the in vitro environment comprises a protein-stabilizing additive, optionally selected from selected from ethylene glycol (EG), 2,2,2-trifluoroethanol (TFE), and deuterated TFE (TFE-d2), or any combination thereof;
(xiii) the protein-stabilizing additive if present optionally has a concentration ranging from about 5% (v/v) to about 50% (v/v) in the in vitro environment;
(xiv) the protein-stabilizing additive if present optionally has a concentration ranging from about 20% (v/v) to about 30% (v/v) in the in vitro environment;
(xv) the method comprises an in vitro pulldown assay to detect the MYC:TRRAP complex and/or the MYC:TRRAP binding interaction, optionally wherein the in vitro pulldown assay comprises the MYC:TRRAP complex formed from the MYC-TRRAP fusion, wherein the MYC-TRRAP fusion comprises at least one affinity tag;
(xvi) the MYC-TRRAP fusion in the MYC:TRRAP complex comprises a MYC 1-190 fragment, a linker, a TRRAP 2033-2088 fragment, and an affinity tag;
(xvii) the method comprises proteolytic cleavage of the MYC:TRRAP fusion at a protease cleavage site within the linker, optionally wherein the protease cleavage site is a 3C protease cleavage site;
(xviii) the method comprises a nuclear magnetic resonance (NMR) assay to detect the MYC:TRRAP complex and/or the MYC:TRRAP binding interaction; optionally wherein the NMR assay detects the MYC:TRRAP complex formed from the MYC-TRRAP fusion, 1H, 15N-HSQC NMR; and one or more chemical shift peaks indicative of a chemical environment of MYC W135, wherein at least one of the chemical shift peaks are different when the MYC:TRRAP binding interaction is present compared to when the MYC:TRRAP binding interaction is absent;
(xix) the MYC-TRRAP fusion comprises a MYC 120-161 fragment, a linker, and a TRRAP 2033-2088 fragment;
(xx) the method further comprises measuring the intrinsic fluorescence of MYC W135 to detect the MYC:TRRAP complex and/or the MYC:TRRAP binding interaction, and wherein the intrinsic fluorescence of MYC W135 is different when the MYC:TRRAP binding interaction is present compared to when the MYC:TRRAP binding interaction is absent;
(xxi) the method further comprises in silico computational analysis of the MYC:TRRAP complex and/or the MYC:TRRAP binding interaction;
(xxii) the cell-based protein-fragment complementation assay is a biomolecular fluorescence complementation (BiFC) assay;
(xxiii) the method includes size exclusion chromatography to detect the MYC:TRRAP complex and/or the MYC:TRRAP binding interaction;
(xxiv) the method includes bioluminescence resonance energy transfer (BRET) to detect the MYC:TRRAP complex and/or the MYC:TRRAP binding interaction;
(xxv) the method includes the use of fluorescence resonance energy transfer (FRET) to detect the MYC:TRRAP complex and/or the MYC:TRRAP binding interaction;
(xxvi) the method includes fluorescence polarization (FP) and/or fluorescence anisotropy (FA) to detect the MYC:TRRAP complex and/or the MYC:TRRAP binding interaction;
(xxvii) the method includes surface plasmon resonance (SPR) to detect the MYC:TRRAP complex and/or the MYC:TRRAP binding interaction;
(xxviii) the method includes native polyacrylamide gel electrophoresis (PAGE) to detect the MYC:TRRAP complex and/or the MYC:TRRAP binding interaction;
(xxix) the method includes the use of a protein microarray to detect the MYC:TRRAP complex and/or the MYC:TRRAP binding interaction;
(xxx) the method includes the use of a microfluidic assay to detect the MYC:TRRAP complex and/or the MYC:TRRAP binding interaction;
(xxxi) the method includes the use of electron microscopy to detect the MYC:TRRAP complex and/or the MYC:TRRAP binding interaction;
(xxxii) the method includes any combination of (i) to (xxxi).

11. The method of claim 1, further comprising:
(i) derivatizing the identified chemical compound to produce a derivatized inhibitor and testing the derivatized inhibitor for an ability to inhibit a binding interaction between MYC and TRRAP; and
(ii) testing the inhibitor or the derivatized inhibitor of the interaction of MYC and TRRAP for its ability to treat cancer or kill tumor cells in an in vitro and/or in vivo tumor model.

12. A method for treating a subject having at least one cancer or precancer or a subject at increased risk of cancer, optionally because of a genetic risk factor, previous cancer and/or expression of a biomarker correlated to cancer, comprising administering to said subject a therapeutically effective amount of the chemical compound identified according to the method of claim 1.

13. The method of claim 12, wherein the subject is a human.

14. (canceled)

15. (canceled)

16. The method of claim 12, wherein the at least one cancer is selected from one or more of adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures.

17.-25. (canceled)
